SEQUENCE LISTING (4 sequences)

SEQ ID NO: 1 - protein, 10 aa, Artificial Sequence
  Peptides derived from OEP34/TOC34 outer membrane protein
MFAFQYLLVM

SEQ ID NO: 2 - protein, 10 aa, Artificial Sequence
  Peptides derived from OEP64/TOC64 outer membrane protein
VILGLGLAGI

SEQ ID NO: 3 - protein, 18 aa, Artificial Sequence
  OEP34 protein transmembrane domain
LIPLMFAFQYLLVMKPLV

SEQ ID NO: 4 - protein, 21 aa, Artificial Sequence
  OEP64 protein transmembrane domain
ASPSSQIWVILGLGLAGIYVL

(19) United States
(12) Patent Application Publication
(10) Pub. No.: US 2024/0148871 A1
(43) Pub. Date: May 9, 2024

(54) SPONTANEOUS RESPIRATORY BIOMATERIAL FOR TISSUE ENGINEERING

(71) Applicant: IUCF-HYU (INDUSTRY-UNIVERSITY COOPERATION FOUNDATION HANYANG, Seoul (KR)

(72) Inventors: Dong Yun LEE, Seoul (KR); Seon Mi JANG, Chungcheongnam-do (KR); Chae Rim YOO, Seoul (KR); Ji Yun Kim, Seoul (KR)

(73) Assignee: IUCF-HYU (INDUSTRY-UNIVERSITY COOPERATION FOUNDATION HANYANG, Seoul (KR)

(21) Appl. No.: 18/282,694

(22) PCT Filed: Mar. 18, 2022
(86) PCT No.: PCT/KR2022/003838

§ 371 Date: Sep. 18, 2023

(30) Foreign Application Priority Data
Mar. 19, 2021 (KR) 10-2021-0035982

(51) Int. Cl. A61K 41/00; A61K 9/06; A61K 35/39; A61K 36/00; A61K 38/16; A61K 38/28; A61K 47/61

(52) U.S. Cl. CPC A61K 41/0057; A61K 9/06; A61K 35/39; A61K 36/00; A61K 38/168; A61K 38/28; A61K 47/61

(57) ABSTRACT

The present invention relates to a hydrogel composition comprising a chloroplast, a chloroplast transit peptide, alginate, and a pancreatic cell, and can supply oxygen to a cell or a tissue and control carbon dioxide and exhibits excellent preventive or therapeutic effects on diseases such as diabetes, etc., and thus may be utilized in the tissue engineering field such as organ transplant.

### TECHNICAL FIELD

The present invention relates to a spontaneous respiratory biomaterial for use in tissue engineering.

### BACKGROUND ART

Oxygen-delivering blood accounts for about 7 to 8% of a body weight and losing at least one-fifth thereof may be life-threatening. The reason why loss of blood through various diseases such as arteriosclerosis, cerebral infarction, myocardial infarction, etc., or vascular injury makes life dangerous is due to lack of oxygen caused by loss of oxygen delivery power. In addition, an organ extracted for organ transplantation is damaged due to hypoxia, ischemia/reperfusion (I/R) damage, and the like, and viability is rapidly reduced due to the lack of oxygen at a site where cell therapies for in vivo injection are injected, and thus a large amount of cell therapies need to be injected into the body (a rapid increase in marginal cell mass). Furthermore, carbon dioxide, which is a by-product of cellular respiration, rapidly affects pH change inside and outside cells and affinity to oxygen, and thus the control of carbon dioxide in a microenvironment around the cells is also an important factor.

Until now, two materials have been developed for oxygen delivery. A first one is an inorganic chemical-based oxygen-releasing biomaterial (ORB), and a second one is an oxygen-generating biomaterial (OBB). However, the materials have a limitation in clinical application due to issues such as (1) cell damage caused by generation of active oxygen species due to initial massive oxygen release, (2) a limited duration of oxygen supply, (3) inability to control carbon dioxide, (4) biocompatibility, etc.

Transplantation of cell-based devices is generally hindered by inadequate oxygen delivery dye to an inevitable delay in angiogenesis after transplantation. Insufficient oxygen supply in the transplanted cells may lead to cell necrosis and cell death in a part of a graft, as well as a conversion into anaerobic metabolism and energy conservation.

It is particularly difficult to provide sufficient oxygen delivery to an implant including cells with high metabolic activity, such as cells including an islet, because an oxygen consumption rate of the islet is high compared to many other cell types, as well as sensitive to dysfunction even at moderate oxygen tension.

Thus, the development of a method for increasing oxygen availability in a tissue engineering implant during an initial period of engraftment may help to mitigate hypoxia-induced cell death. In situ oxygen generation is a highly desirable approach in that the generation does not require several operations and provides supplemental oxygen immediately upon implantation. These approaches highlight the potential of in situ oxygen generation to improve cell viability, but most of the approaches make transplantation difficult by increasing a size of a transplant device or by introducing a toxic by-product.

In conclusion, for the development of oxygen-generating biomaterials, it is necessary to reduce sensitivity in producing side reactions such as hydrogen peroxide intermediates or hydroxyl radicals by eliminating end products with cytotoxicity and controlled reactivity.

Thus, in order to overcome the above limitations, there is a need to develop a spontaneous respiratory material for tissue engineering, which is capable of supplying oxygen and controlling carbon dioxide and active oxygen.

### DISCLOSURE

### Technical Problem

One aspect is to provide a hydrogel composition including a chloroplast and a chloroplast transit peptide (CTP).

Another aspect is to provide a hydrogel composition including a chloroplast, a chloroplast transit peptide (CTP) and alginate.

A further aspect is to provide a hydrogel composition including a chloroplast, a chloroplast transit peptide (CTP), alginate and a pancreatic cell.

A yet further aspect is to provide a pharmaceutical composition for preventing or treating diabetes, including the hydrogel composition.

A still yet further aspect is to provide a microcapsule including the hydrogel composition.

A still yet further aspect is to provide a method for preparing a hydrogel composition, the method including: binding alginate and a chloroplast transit peptide (CTP); and
mixing the bound alginate-CTP composite with a chloroplast isolated from an individual to gelate the resulting mixture.

Another aspect is to provide a method for delivering oxygen to a cell or a tissue,
the method including: treating the hydrogel composition; and irradiating the treated hydrogel composition with light.

### Technical Solution

In order to overcome the limitations of the related art, the present inventors have developed a spontaneous respiratory material for tissue engineering, which is capable of supplying oxygen and controlling carbon dioxide and active oxygen.

Hereinafter, the present invention will be described in more detail.

Terms used in the present application are used only to describe a certain exemplary embodiment and are not intended to limit the present invention. All the terms used herein including technical or scientific terms have the same meaning as commonly understood by those ordinary skilled in the art, to which the present invention pertains, unless defined otherwise.

The present invention may provide a hydrogel composition including a chloroplast and a chloroplast transit peptide (CTP).

In the present specification, the term “respiratoid” may refer to a sustainable biomaterial of the present invention, which is a coined word made by combining “respirate” and a suffix of “˜oid” which means “artificiality.” The respiratoid may rapidly regenerate carbon dioxide, which is produced by cells/organs through cellular respiration, into oxygen, may increase a production rate thereof when being exposed to a specific wavelength, and may regenerate active oxygen (superoxide anion, hydrogen peroxide, etc.) generated from cells or tissues into oxygen again in the case of a temporary hypoxia state. In addition, the respiratoid according to the present invention may be highly biocompatible and thus may have little toxicity.

Respiratoid may have “excellence/differentiation” as summarized in a table below compared to existing oxygen-releasing materials (ORB) and oxygen-generating materials (OBG).

TABLE 1

| | Oxygen- | Oxygen- | |
|---|---|---|---|
| | releasing | generating | |
| | biomaterials | biomaterials | |
| Classification | (ORB) | (OGB) | Respiratoid |
| Example | Sodium | Perfluorooctance | Respiratoid |
| | percarbonate | | (Present Proposal) |
| | Calcium | | |
| | peroxide | | |
| | Magnesium | | |
| | peroxide | | |
| External | Unnecessary | Unnecessary | Unnecessary/Required |
| stimulus | | | |
| (pH, light, | | | |
| heat) | | | |
| By-product | Active | None | None |
| | oxygen | | |
| O₂ supply | Several | Several hours | Infinite |
| | hours | to days | |
| CO₂ removal | None | None | Infinite |
| Cytotoxicity | Present | Present | None |

In one embodiment of the present invention, the hydrogel composition may be a respiratoid, which is a spontaneous respiratory material for tissue engineering, including a chloroplast and CTP. Thus, when the hydrogel composition is used, carbon dioxide generated by cells/organs through cellular respiration may be rapidly regenerated back into oxygen.

In one embodiment of the present invention, the hydrogel composition may use chloroplasts present in plant leaves, and alginate used for gelation may be obtained from plants such as seaweed leaves, etc. In addition, the chloroplast transit peptide having high selectivity for an outer membrane among the two layers of chloroplast composed of fatty acids is used rather than the chloroplast per se in a modification reaction, thereby having excellent biocompatibility and less influence on photoreaction of chloroplasts, Calvin-circuit reaction, etc.

Furthermore, the hydrogel composition may supply oxygen supply and control carbon dioxide, and thus may be highly likely to be widely used in the field of tissue engineering such as blood cell therapies, organ transplantation, and the like. Specifically, there may be provided very high originality and versatility, as well as utilization in the fields of (1) artificial blood development, (2) organ extraction and preserving agents, (3) large-capacity culture of microorganisms/cells with high efficiency, (4) (stem) cell therapies and tissue engineering, and the like. In addition, research on an in vivo cell-cell interaction may be possible in a biomimetic environment, so that a new function of cells may be examined in terms of physiology, and may be applied as a new technique in a field of research on stem cell therapy.

In one embodiment of the present invention, the hydrogel composition may use a stromal calvin cycle and thylakoid present in a chloroplast of plant leaves, which simultaneously controls oxygen and carbon dioxide. For this purpose, the hydrogel composition may include a chloroplast transit peptide (CTP) capable of binding or conjugating to the chloroplast. In one specific embodiment, the CTP may bind to a chloroplast outer membrane.

In the present invention, “binding” may include chemical binding and physical binding. For example, the CTP may be physically conjugated to or inserted into the chloroplast outer membrane, and the CTP may bind to the chloroplast outer membrane through covalent bonding.

In one embodiment of the present invention, the CTP may be derived from a chloroplast membrane protein. For example, the CTP may include a partial sequence of the chloroplast membrane protein, and specifically may include a partial sequence of the chloroplast outer envelope protein.

In one embodiment of the present invention, the CTP may be derived from outer envelope protein 34 (OEP34) of chloroplasts (or translocon at the outer envelope membrane of chloroplasts 34 (TOC34, Arabidopsis thaliana sp.)) or outer envelope protein 64 (OEP64) (or translocon at the outer envelope membrane of chloroplasts 64 (TOC64, Arabidopsis thaliana sp.)). In one specific embodiment, the CTP may be derived from OEP34/TOC34 or OEP64/TOC64.

In one embodiment of the present invention, the CTP may include a part of a sequence of a transmembrane domain of chloroplast outer envelope protein 34 or outer envelope protein 64. For example, the CTP may be a peptide including a part of one or more sequences selected from amino acid sequences of transmembrane domains represented by SEQ ID NO: 3 and SEQ ID NO: 4.

In one embodiment of the present invention, the CTP may be a peptide including one or more selected from amino acid sequences represented by SEQ ID NO: 1 and SEQ ID NO: 2.

In one embodiment of the present invention, the CTP may be a peptide including MFAFQYLLVM (SEQ ID NO: 1), which may be a peptide sequence derived from LI PLMFAFQYLL VMKPLV (SEQ ID NO: 3), which is a predicted sequence of a transmembrane domain of chloroplast outer envelope protein OEP34 (or TOC34).

In one embodiment of the present invention, the CTP may be a peptide including VILGLGLAGI (SEQ ID NO: 2), which may be a peptide sequence derived from SPSSQ IWVILGLGLA GIYVL (SEQ ID NO: 4), which is a predicted sequence of a transmembrane domain of chloroplast outer envelope protein OEP64 (or TOC64).

In one embodiment of the present invention, the CTP may increase oxygen generation of chloroplasts.

In one embodiment of the present invention, the hydrogel composition may further include alginate.

In one embodiment of the present invention, the hydrogel may be gelated by binding a CTP terminus and an alginate terminus. In one specific embodiment, the hydrogel may be gelated by conjugating or binding primary amine of the CTP and a carboxyl group of alginate. In addition, in one specific embodiment, the alginate may be mixed with a calcium solution and gelated through cross-linking.

In one embodiment of the present invention, the hydrogel may further include pancreatic cells. In one specific embodiment, the pancreatic cells may be encapsulated in a chloroplast-CTP-alginate hydrogel. A chloroplast-CTP-alginate hydrogel composition of the present invention, in which pancreatic cells are encapsulated, may exhibit excellent cell viability and a preventive or therapeutic effect on diabetes according to spontaneous respiration, and thus may be applied to a hydrogel-based implant for tissue engineering using the same.

In one embodiment of the present invention, the hydrogel composition may generate oxygen during light irradiation.

In one embodiment of the present invention, the light irradiation may be performed at an oxygen concentration of 101/ml to 1010/ml. In one specific embodiment, the light irradiation may be performed at an oxygen concentration of 102/ml to 108/ml.

In one embodiment of the present invention, the light irradiation may be performed at a temperature of 20° C. to 40° C. In one specific embodiment, the light irradiation may be performed at a temperature of 23° C. to 38° C.

In one embodiment of the present invention, the light irradiation may be continuously performed for a predetermined time, or may be non-continuously and alternately performed by exposing light for a predetermined time and not exposing light at predetermined time intervals.

In one embodiment of the present invention, the light irradiation may be performed once to five times by continuously exposing light for 10 to 50 minutes, and then not exposing light for 10 to 50 minutes in a cycle. In one specific embodiment, the light irradiation may be performed twice to four times by continuously exposing light for 20 to 40 minutes, and then not exposing light for 20 to 40 minutes in a cycle.

In one specific embodiment, an oxygen generation rate of the hydrogel composition may be increased upon exposure to a specific wavelength, and the wavelength may be 620 nm to 700 nm, 640 nm to 680 nm, or 650 nm to 670 nm.

In addition, in one specific embodiment, the hydrogel composition may regenerate active oxygen, which is generated when surrounding cells or tissues are in a temporary hypoxia state, into oxygen.

In one embodiment of the present invention, the hydrogel may include chloroplasts at a concentration of 101/ml to 1010/ml. In one specific embodiment, the hydrogel may include chloroplasts at a concentration of 103/ml to 1010/ml, 106/ml to 1010/ml, 107/ml to 109/ml, or 103/ml to 109/ml.

In one embodiment of the present invention, the hydrogel may include insulin secreted by pancreatic cells. The pancreatic cells included in the hydrogel composition of the present invention may be present in a state of being encapsulated in the hydrogel, thereby stably secreting insulin.

In one embodiment of the present invention, the hydrogel composition may be for delivering oxygen to a cell or a tissue. In one specific embodiment, the oxygen delivery may be performed by rapidly regenerating carbon dioxide produced through cellular respiration back into oxygen, or by reproducing active oxygen, which is generated from cells or tissues in a temporary hypoxia state, back into oxygen. The hydrogel composition of the present invention may alleviate cell death by oxygen delivery, and may control an oxygen generation rate.

In addition, the present invention may provide a microcapsule including the hydrogel composition. The microcapsule may be used in a cell therapeutic agent.

In one embodiment of the present invention, the microcapsule may be a microcapsule for generating oxygen during light irradiation. In addition, in one embodiment, the microcapsule may be a microcapsule for preventing or treating metabolic diseases.

Furthermore, the present invention may provide a pharmaceutical composition for preventing or treating metabolic diseases, including the hydrogel composition.

In the present specification, the term “prevention” may refer to all the actions of inhibiting a disease or delaying the onset of the disease in an individual by administration of the pharmaceutical composition according to one aspect.

In the present specification, the term “treatment” may refer to all the actions of ameliorating or beneficially changing symptoms of a disease by administration of the pharmaceutical composition according to one aspect.

In addition, “active ingredient” or “pharmaceutically effective amount” may refer to any amount of a composition used in a course of practicing the invention provided herein, to be sufficient for alleviating, inhibiting a progression of, or preventing a disease, disorder, or condition, or one or more symptoms thereof.

According to one embodiment of the present invention, the metabolic disease may be one or more selected from the group consisting of type 1 diabetes, type 2 diabetes, impaired glucose tolerance, impaired fasting glucose, dyslipidemia, impaired lipid metabolism, obesity, fatty liver, insulin resistance, and glucose tolerance syndrome. In one specific embodiment, the metabolic disease may be diabetes, and may be type 1 diabetes or type 2 diabetes.

The pharmaceutical composition may further include a pharmaceutically acceptable carrier or diluent. Pharmaceutically acceptable carriers or diluents may be those known in the art. The carrier or diluent may be lactose, dextrose, sucrose, sorbitol, mannitol, starch, gum acacia, calcium phosphate, alginate, gelatin, calcium silicate, microcrystalline cellulose, polyvinylpyrrolidone, cellulose, water (e.g., saline and sterile water), syrup, methyl cellulose, methylhydroxybenzoate, propylhydroxybenzoate, talc, magnesium stearate, mineral oil, Ringer's solution, buffering agent, maltodextrin solution, glycerol, ethanol, dextran, albumin, or any combination thereof. The pharmaceutical composition may further include a lubricant, humectant, a sweetener, a flavoring agent, an emulsifying agent, a suspending agent, or a preservative.

The pharmaceutical composition may be formulated using a pharmaceutically acceptable carrier and/or excipient according to methods known to those skilled in the art to prepare a unit dose form or may be incorporated into a multi-dose container. In this case, a formation may be in a form of solution, suspension, syrup, or emulsion in oil or aqueous solvent, or may be in a form of extract, powder, granule, tablet, or capsule, and may further include a dispersant or a stabilizer. The aqueous solvent may include physiological saline or PBS. The pharmaceutical composition according to one specific embodiment may be formulated into an oral or parenteral dosage form, preferably a parenteral dosage form. In the case of intramuscular, intraperitoneal, subcutaneous, and intravenous dosage forms, a sterile solution of the active ingredient may be conventionally prepared to include a buffer capable of suitably adjusting a pH of the solution, and in the case of intravenous administration, an isotonic agent may be included to impart isotonicity to the preparation.

A dosage (effective amount) of the pharmaceutical composition according to one specific embodiment may be variously prescribed depending on factors such as a formulation method, an administration method, a patient's age, weight, gender, pathologic condition, food, administration time, administration route, excretion rate and reaction sensitivity, and those skilled in the art may appropriately adjust the dosage in consideration of these factors. The administration may be performed once a day or twice or more within a range of clinically acceptable side effects, and the administration may be performed at one site or at two sites or more, and the administration may be performed daily or at intervals of two to five days with the total number of administration lasting from one day to 30 days during one treatment. If necessary, the same treatment may be repeated after an appropriate period. Non-human animals may be administered in the same dosage as that of human per kg, or may be administered in an amount obtained by converting the above dosage into, for example, a volume ratio (e.g., an average value) of a target animal and an organ (heart, etc.) of human. Possible routes of administration may include parenteral (e.g., subcutaneous, intramuscular, intraarterial, intraperitoneal, intrathecal, or intravenous), topical (including transdermal), and injection, or insertion of an implantable device or substance. Examples of the animal to be treated according to one specific embodiment may include humans and other target mammals, and specifically include humans, monkeys, mice, rats, rabbits, sheep, cattle, dogs, horses, pigs, etc.

In addition, the present invention may provide a health functional food for preventing or ameliorating metabolic diseases, including the hydrogel composition as an active ingredient.

The hydrogel composition, metabolic diseases, and prevention may be the same as described above.

In the present specification, the term “amelioration” may refer to all the actions of inhibiting a metabolic disease or delaying the onset of the metabolic disease in an individual by administration of the composition according to one aspect.

The health functional food defined in the present invention may be a health functional food which has sufficiently established the functionality and safety for the human body newly defined through the Health Functional Foods Act amended in 2008 and has been registered in the regulations on the recognition of functional raw materials for health functional foods prescribed in the Food and Drug Administration Notice No. 2008-72 of the Food and Drug Administration.

When the composition of the present invention is used by being included in a health functional food, the composition may be added per se or used together with other health functional foods or health functional food ingredients, and may be appropriately used according to a conventional method. A mixing amount of the active ingredient may be suitably determined according to a purpose of use. In general, the active ingredient constituting the composition according to the present invention may be included in an amount of 0.01 to 15% by weight, preferably 0.2 to 10% by weight, based on the total weight of the food, and when being prepared as a beverage, the active ingredient may be included at a ratio of 0.1 to 30 g, preferably 0.2 to 5 g based on 100 mL, and the entire beverage may be composed of a natural ingredient. However, in the case of long-term intake for the purpose of health control and hygiene, the amount may be equal to or less than the above range, and the active ingredient may be used in an amount equal to or greater than the above range since there is no problem in terms of safety.

The composition for health functional food according to the present invention may be formulated into a dosage form of conventional health functional food known in the art. The health functional food may be prepared, for example, in a form of powder, granule, tablet, pill, capsule, suspension, emulsion, syrup, precipitate, liquid, extract, gum, tea, jelly, drink, or the like, and preferably in a form of drink. As the sitologically acceptable carrier or additive, any carrier or additive known to be usable in the art may be used for preparing a formulation to be prepared. It may also include foods used as feed for animals.

The health functional food may further contain nutritional supplements, vitamin, electrolyte, flavoring agent, coloring agent, pectic acid and salts thereof, alginic acid and salts thereof, organic acid, protective colloidal thickener, pH adjusting agent, stabilizer, preservative, glycerin, alcohol, carbonator used in carbonated beverage, etc., according to purpose or preference. Besides, the health functional food may contain natural fruit juice and pulp for preparing fruit juice beverage and vegetable based beverage. In addition, the health functional food composition may further include a food additive, and the suitability as a “food additive” may be determined according to the specifications and standards for the relevant item in accordance with the general rules, general test methods and the like of the Food Additive Code approved by the Ministry of Food and Drug Safety, unless otherwise specified.

Furthermore, the present invention may provide a method for preparing a hydrogel composition, the method including: binding alginate and a chloroplast transit peptide (CTP); and mixing the bound alginate-CTP composite with a chloroplast isolated from an individual to gel a resulting mixture.

In one embodiment of the present invention, the preparation method may further include: encapsulating pancreatic cells inside the gelled hydrogel.

In addition, the present invention may provide a method for delivering oxygen to a cell or a tissue, the method including: treating the hydrogel composition; and irradiating the treated hydrogel composition with light.

In one embodiment of the present invention, the light irradiation may be performed at an oxygen concentration of 101/ml to 1010/ml. In one specific embodiment, the light irradiation may be performed at an oxygen concentration of 102/ml to 108/ml.

In one embodiment of the present invention, the irradiating with light may be performed at a temperature of 20° C. to 40° C. In one embodiment, the irradiating with light may be performed at a temperature of 23° C. to 38° C.

In one embodiment of the present invention, the irradiating with light may be continuously performed for a predetermined time, or may be non-continuously and alternately performed by exposing light for a predetermined time and not exposing light at predetermined time intervals.

In one embodiment of the present invention, the irradiating with light may be performed once to five times by continuously exposing light for 10 to 50 minutes, and then not exposing light for 10 to 50 minutes in a cycle. In one specific embodiment, the irradiating with light may be performed twice to four times by continuously exposing light for 20 to 40 minutes, and then not exposing light for 20 to 40 minutes in a cycle.

In one specific embodiment, an oxygen production rate according to the method may be increased upon exposure to a specific wavelength, and the wavelength may be 620 nm to 700 nm, 640 nm to 680 nm, or 650 nm to 670 nm.

In addition, the present invention may provide a method for preventing or treating a metabolic disease, the method including: administering the hydrogel composition to an individual. In one embodiment, the method may further include: irradiating the individual with light.

In addition, the present invention may provide a use of the hydrogel composition.

Furthermore, the present invention may provide a use of the hydrogel composition for use in preparing a medicament for the prevention or treatment of metabolic diseases.

Moreover, the present invention may provide a use of a microcapsule including the hydrogel composition.

Besides, the present invention may provide a use of a microcapsule including the hydrogel composition for use in preparing a medicament for the prevention or treatment of metabolic diseases.

### Advantageous Effects

The hydrogel composition including a chloroplast, a chloroplast transit peptide (CTP) and alginate of the present invention can supply oxygen to a cell or a tissue and control carbon dioxide.

The hydrogel composition including a chloroplast, a chloroplast transit peptide (CTP), alginate, and pancreatic cells of the present invention can be harmless to cells and individuals, and can exhibit an excellent effect of preventing or treating diseases such as diabetes, etc.

### DESCRIPTION OF DRAWINGS

FIG. 1 is a view showing an action of a sustainable spontaneous respiratory material in cells/organs. Through the present spontaneous respiratory material, carbon dioxide produced in cells through cellular respiration may be regenerated into oxygen again by a photoreaction of chloroplast of the spontaneous respiratory material, and an oxygen production rate may be increased at a specific wavelength (660 nm). In addition, active oxygen generated in a transient hypoxia state may be regenerated into oxygen again through an enzymatic reaction by a catalase (CAT) or superoxide dismutase (SOD) enzyme.

FIG. 2 is a view showing an action of anchoring of alginate-CTP to an outer membrane of chloroplast. A CTP sequence is Met-Phe-Ala-Phe-Gln-Tyr-Leu-Leu-Val-Met (1.26 kDa), which may be anchored to a chloroplast outer membrane to form alginate-CTP-chloroplast.

FIG. 3a is a view showing a chloroplast-transit-peptide (CTP) sequence. FIG. 3b is a view showing a mass spectrometry (MS) result. A chloroplast-transit-peptide (CTP) sequence capable of being inserted into the chloroplast outer membrane was established, and a 1.26 kDa MFAFQYLLVM (SEQ ID NO: 1) peptide with 97% purity was synthesized.

FIG. 4 is a schematic view showing a chemical binding reaction formula between COOH of alginate and N-terminus of peptide using the EDC/NHS method. For the synthesis of CTP-alginate, 20 mM sodium alginate was dissolved in 10 ml of 0.1 M MES buffer at pH 6, and then 2 mM EDC and 5 mM NHS were added thereto and reacted at room temperature for 15 minutes. After adjusting to pH 6, peptide dissolved in 1% DMSO was added at a molar ratio and reacted at room temperature for two hours. After proceeding with dialysis, the resulting product was freeze-dried to obtain a composite. A carbodiimide coupling reaction was used to form an amide bond with carboxylate group of alginate and primary amine of CTP to prepare a conjugate. EDC/NHS was used to increase the efficiency of the carbodiimide coupling reaction.

FIG. 5 is a schematic view showing a ¹H-NMR result of an alginate-CTP composite. As a result of 1H-NMR, in the CTP-alginate composite, an alginate peak was confirmed at around 4 ppm, an amine bond peak of the peptide was confirmed at 2.7 ppm, and an n-acylurea peak of the peptide was confirmed at around 2.9 ppm and 1.2 ppm in all nine groups having a molar ratio of alginate:CTP binding at 1:1 to 1:600.

FIG. 6 is a view showing a result of comparing a molar ratio (1:1, 1:5, 1:10, 1:25, 1:50) at which existing synthesis was performed with a molar ratio at which actual synthesis was performed by quantifying proteins through BCA assay after alginate-CTP synthesis.

FIGS. 7a and 7b are results of confirming gelation of an alginate-CTP composite and a strength of gel (100 mM CaCl₂)) through a rheometer. Conditions for forming a capsule gel of a CTP-alginate conjugate were optimized using a CaCl₂) solution. 400 ul of CaCl₂) solution was added at each molar concentration, and 200 ul of an alginate solution, which is prepared by adding alginate-CTP of 8 mg/ml concentration synthesized at various ratios to 0.9% NaCl, was added thereto and then gelation was performed for 30 minutes so that the capsule gel formation was confirmed.

FIG. 8a is a view showing a chloroplast isolation process. FIG. 8b is a view confirming an isolated chloroplast at 200× magnification of an optical microscope. After isolating 2*1010 total chloroplasts based on 35 g of spinach leaves in the same process as the schematic view of FIG. 8a, conditions for optimized chloroplast isolation were established to obtain a complete chloroplast with a bright round-shaped border.

FIGS. 9a and 9b are a graph (%) and a table showing the results of evaluating the toxicity of DMSO in chloroplast. After treating chloroplast with DMSO, the toxicity confirmed for each time was evaluated (%, n=5), and an amount of chloroplast for each time was confirmed.

FIGS. 10a and 10b are views showing the results of confirming an anchoring ability by a confocal microscope and TEM after treating chloroplast with CTP-FITC. In order to evaluate the anchoring ability between CTP peptide and chloroplast, 1*108/ml of chloroplast and 0.6 mg/ml of CTP-FITC were treated and analyzed by a confocal microscope and TEM for each time (1, 5, 10, 30, 60 min).

FIG. 11 shows a photograph of hydrogel formed of CaCl₂ solution after preparing a conjugate by treating chloroplast and alginate-CTP synthesized at various molar ratios, as well as a confirmed remaining amount of chloroplast after forming the hydrogel, and a graph showing a chlorophyll encapsulation ratio according to a feed molar ratio of alginate-CTP synthesis. Prior to the present experiment, a gelation test was performed as a preceding experiment to confirm whether anchoring of alginate-CTP and chloroplast may not affect gelation or not even if CIB was used, and it was confirmed that gelation does not occur in CIB in the same manner as in PBS, which is a generally used buffer. FIG. 11a shows a photograph (left) of a capsule gel formed of a CaCl₂) solution after preparing a composite by treating chloroplast and alginate-CTP synthesized at various molar ratios of the prepared capsule gel, and a result (right) of confirming a remaining amount of chloroplasts after forming the capsule gel. After preparing 200 μl of a solution obtained by treating an alginate-CTP conjugate having a feed molar ratio of 1:1 to 1:50 with 1*108/ml of chloroplast and reacting for five minutes, 400 μl of 100 mM CaCl₂) solution was added thereto, and 200 μl of alginate solution prepared by adding alginate-CTP of 8 mg/ml concentration synthesized at various ratios to 0.9% NaCl, and gelation was performed for 30 minutes to form hydrogel. FIG. 11b is a result of confirming an amount of chloroplast bound into hydrogel by measuring a concentration of total chlorophyll.

FIG. 12a is a photograph taken with an optical microscope after treatment of HEK293 cell at each concentration of chloroplast, and FIG. 12b is a graph (%, n=5) showing a result of cell viability assay (CCK-8 assay). (N.S.: statistically no significant, *P≤0.05, **P≤0.01, ***P≤0.001, n=5, Results are shown as mean±S.E.M)

FIG. 13a is a view showing a state in which light (660 nm) for enhancing photosynthesis efficiency of chloroplast is exposed to a chloroplast solution with a distance of 5 cm from the ground. FIG. 13b is a graph comparing an amount of oxygen generated according to a concentration of chloroplast depending on the presence or absence of light in a normoxic state.

FIG. 14 is a photograph (14a) of alginate-CTP-chloroplast hydrogel (ACC) according to a concentration of chloroplast and a graph (14b) comparing an amount of oxygen generated according to a concentration of chloroplast in the ACC depending on the presence or absence of light in a normoxic state. As shown in FIG. 14a, it was confirmed that hydrogel having a relatively uniform size of about 0.5 cm is produced while well maintaining a shape, and a concentration of color becomes higher as a concentration of chloroplast increases.

FIG. 15a is a view showing alginate-CTP-chloroplast (ACC) and alginate-chloroplast hydrogel (AC) prepared at each concentration of chloroplast. FIG. 15b is a graph showing a result of performing a rheology test to confirm a long-term strength of alginate-CTP-chloroplast hydrogel depending on the presence or absence of CTP. There was no group-to-group difference on day 0, but a higher storage modulus value was measured on hydrogel with CTP on day 7. On day 30, the storage modulus values of all groups were measured close to zero. Thus, an exact mechanism has not yet been identified, but it was confirmed that hydrogel with CTP shows better physical strength (properties).

FIG. 16 is a graph showing a result of measuring an amount of oxygen generated from alginate-CTP-chloroplast hydrogel at each concentration of chloroplast according to a normoxic state and a hypoxic state.

FIG. 17 is a graph showing a result of measuring an amount of oxygen generated from free chloroplast, alginate-chloroplast (AC) hydrogel, and alginate-CTP-chloroplast (ACC) hydrogel upon light exposure in a hypoxic state. FIG. 17a is a graph (μM/L-left, %-right, n=5) showing an amount of oxygen generated from free chloroplast upon light exposure in a hypoxic state. FIG. 17b is a graph (μM/L-left, %-right, n=5) showing an amount of oxygen generated from the AC upon light exposure in a hypoxic state. FIG. 17c is a graph (μM/L-left, %-right, n=5) showing an amount of oxygen generated from the ACC upon light exposure in a hypoxic state.

FIG. 18 is a graph showing a result of performing ABTS assay to confirm a reason why an amount of oxygen generated decreases in a group with a high concentration of chloroplast.

FIG. 18a is a graph (n=5) showing an amount of hydrogen peroxide generated from free chloroplast. FIG. 18b is a graph (n=5) showing an amount of hydrogen peroxide generated from alginate-CTP-chloroplast hydrogel.

FIG. 19 is a graph showing a result of cell viability assay (CCK-8 assay) at each concentration of alginate-CTP solution with regard to INS-1 cells (single cells) as well as a graph showing a result of live & dead assay. FIG. 19a is a graph (%, n=5) quantitatively representing the viability of INS-1 cells according to a concentration of alginate-CTP solution. FIG. 19b is a view showing a live & dead assay confirmed through a fluorescent micrograph (200×, green fluorescence—live cells, red fluorescence—dead cells), which qualitatively shows the viability of INS-1 cells according to a concentration of alginate-CTP solution. (N.S.: statistically no significant, *P≤0.05, **P≤0.01, ***P≤0.001, n=5, Results are shown as mean±S.E.M)

FIG. 20 is a graph showing a result of cell viability assay (CCK-8 assay) for each concentration of alginate-CTP hydrogel with regard to pancreatic cells (cell group) as well as a graph showing a result of live & dead assay. FIG. 20a is an optical micrograph (40×) of pancreatic cells encapsulated in alginate-CTP-chloroplast hydrogel at each chloroplast concentration. FIG. 20b is a graph (%, n=5) quantitatively representing the viability of pancreatic cells according to a concentration of alginate-CTP hydrogel. FIG. 20c is a fluorescent micrograph (200×, green fluorescence—live cells, red fluorescence—dead cells) qualitatively representing the viability of pancreatic cells according to a concentration of alginate-CTP hydrogel. (N.S.: statistically no significant, *P≤0.05, **P≤0.01, ***P≤0.001, n=5, Results are shown as mean±S.E.M)

FIG. 21 is a graph showing a result of cell viability assay (CCK-8 assay) of pancreatic cells encapsulated in alginate-CTP-chloroplast hydrogel with chloroplast treated at each concentration as well as a picture showing a result of live & dead assay. FIG. 21a is an optical micrograph (40×) of pancreatic cells encapsulated in alginate-CTP-chloroplast hydrogel at each concentration of chloroplast. FIG. 21b is a graph (%, n=5) quantitatively representing the viability of pancreatic cells encapsulated in alginate-CTP-chloroplast hydrogel at each concentration of chloroplast. FIG. 21c is a fluorescent micrograph (200×, green fluorescence—live cells, red fluorescence—dead cells) qualitatively representing the viability of pancreatic cells encapsulated in alginate-CTP-chloroplast hydrogel at each concentration of chloroplast. (N.S.: statistically no significant, *P≤0.05, **P≤0.01, ***P 0.001, n=5, Results are shown as mean±S.E.M)

FIG. 22 is a graph showing a result of measuring for 30 hours a concentration of oxygen in alginate-CTP-chloroplast hydrogel with pancreatic cells encapsulated therein and with chloroplast treated at each concentration. FIG. 22a is a graph (μM/L, n=5) showing a measured amount of oxygen generated from alginate-CTP-chloroplast hydrogel with pancreatic cells encapsulated therein at each concentration of chloroplast. FIG. 22b is a graph (based on 24 hours) showing a measured amount of oxygen generated from alginate-CTP-chloroplast hydrogel with pancreatic cells encapsulated therein at each concentration of chloroplast. (N.S.: statistically no significant, *P≤0.05, **P≤0.01, ***P≤0.001, n=5, Results are shown as mean±S.E.M)

FIG. 23 is a graph showing a result of evaluating an insulin secretion ability of pancreatic cells encapsulated in alginate-CTP-chloroplast hydrogel with chloroplast treated at each concentration (GSIS assay) as well as a graph (insulin ng/DNA ng, n=5) showing a glucose stimulation index of pancreatic cells accordingly.

FIG. 24 is a view showing an apparatus capable of inducing a hypoxic environment for accurately measuring an amount of oxygen generated. A concentration of oxygen was measured in real time by adding an oxygen plate reader (SDR SensorDish Reader, PreSens) in a hypoxia chamber, and a temperature condition (37° C.) and an environment which may be exposed to light (660 nm, 2000 lux, distance: 15 cm from sample) were implemented in oven, and an inflow was blocked using a hypoxia chamber sealed parafilm to prevent an inflow of oxygen from outside. Mixed gas (1% O2+5% CO2+94% N2) was injected at a rate of 40 L/min into the hypoxia chamber in which the surrounding environmental conditions were implemented to implement a hypoxic environment of 1% O2.

FIG. 25 is a graph showing a result of measuring an amount of oxygen generated from chloroplast (106/ml, 107/ml, 108/ml) depending on the presence or absence of a light (660 nm) condition. After a hypoxic environment was induced using the hypoxia chamber implemented in above FIG. 24, fresh isolated chloroplast diluted in media (RPMI+1% PS, 10% FBS) was prepared at each concentration of 106/ml, 107/ml, and 108/ml to put a chloroplast solution into a hypoxic environment (60 mmHg or less), and then an oxygen plate reader was put into the hypoxia chamber to continuously measure an oxygen concentration of chloroplast solution for 20 hours. In addition, in order to measure a degree of oxygen generation depending on the presence or absence of light (660 nm), an experiment was performed by dividing chloroplasts into a group (light) of being exposed to light for 20 hours and a group (no light) of not being exposed to light. FIG. 25a is a result (mmHg, n=6) of measuring an amount of oxygen generated from chloroplast at each concentration depending on the presence or absence of a light (660 nm) condition. FIGS. 25b (mmHg, n=6) and 25c (%, n=6) are results of measuring an amount of oxygen generated from chloroplast at each concentration depending on the presence or absence of a light (660 nm) condition.

FIG. 26 is a schematic view showing a temperature-controlled desiccator capable of inducing a hypoxic environment to compensate for the disadvantages of the hypoxia chamber of FIG. 24. FIG. 26a is a view showing a hypoxic environment induction device using a temperature-controlled desiccator. FIG. 26b is a view showing a result of confirming a hypoxic environment state and a hypoxic environment induction device using a temperature-controlled desiccator. A concentration of oxygen was measured in real time by adding an oxygen plate reader (SDR SensorDish Reader, PreSens) in a temperature-controlled desiccator, and a temperature condition (37° C.) and an environment which may be exposed to light (660 nm, 2000 lux, distance: 15 cm from sample) were implemented. Mixed gas (1% O2+5% CO2+94% N2) was injected at a rate of 40 L/min into a temperature-controlled desiccator in which the surrounding environmental conditions were implemented to implement a hypoxic environment of 1% O2 and the environment may be confirmed in real time using an oxygen detector. As a result of comparing above FIGS. 24 and 26, it was confirmed that an external air inflow blocking ability is excellent when using the desiccator.

FIG. 27 is a graph showing a measured oxygen concentration and a view showing a result of measuring an area under the ROC curve (AUC) when chloroplast is cultured for a long time (20 hours) in a hypoxic environment (desiccator). After a hypoxic environment was induced using the temperature-controlled desiccator implemented in above FIG. 26, fresh isolated chloroplast diluted in media (RPMI+1% PS, 10% FBS) was prepared at each concentration of 108/ml to put a chloroplast solution into a hypoxic state (1% O2), and then an oxygen concentration of chloroplast solution was continuously measured using an oxygen plate reader for 20 hours. FIG. 27a is a result of measuring an oxygen concentration when chloroplast was cultured in a hypoxic environment (desiccator) for a long time (20 hours) (torr-left, %-right, n=24). FIG. 27b is an AUC result of measuring an oxygen concentration when chloroplast was cultured in a hypoxic environment (desiccator) for a long time (20 hours) (min*torr, n=24).

FIG. 28 is a schematic view showing a comparison between a hypoxia chamber (A) and a desiccator (B). If an external oxygen inflow is prevented using the hypoxia chamber sealed parafilm of FIG. 28(A), an oxygen concentration (40 mmHg) may be maintained at a hypoxic environment level for 20 hours. However, there may be disadvantages in that 40 L of mixed gas (1% O2+5% CO2+94% N2) needs to be used to create a hypoxic environment, a long time is required, two or more oxygen plate readers cannot be used, and an oxygen detector cannot be inserted, and thus the hypoxic environment needs to be determined by an oxygen concentration of solution rather than an environment in a hypoxia chamber. The temperature-controlled desiccator of FIG. 28(B) may adjust a temperature per se, thus allowing an experiment to be performed in various temperature environments, and may have a size of 84 L and have a size larger than the hypoxia chamber, thus having an advantage in that it is possible to insert a light, two or more oxygen plate readers and an oxygen detector therein. In addition, the desiccator may have a completely sealed environment due to properties of the desiccator, and may have an advantage in that an oxygen concentration inside the desiccator may be detected in real time. Considering the above two, the use of the temperature-controlled desiccator may be considered to be advantageous in inducing a hypoxic environment and then conducting an experiment, rather than using the hypoxia chamber, and thus the experiment was conducted using the desiccator in a later study.

FIG. 29 is a graph showing a result of measuring an oxygen concentration in a hypoxic environment of chloroplast according to a temperature condition. After a hypoxic environment is induced using the temperature-controlled desiccator implemented in above FIG. 26, fresh isolated chloroplast diluted in media (RPMI+1% PS, 10% FBS) may be prepared at 108/ml to put a chloroplast solution into a hypoxic environment (1% O2), and then an oxygen concentration of chloroplast solution may be continuously measured using an oxygen plate reader for 20 hours. At this time, chloroplast may be diluted in media (RPMI+1% PS, 10% FBS) pre-heated at 25° C. and 37° C., respectively, in order to measure an oxygen generation ability of chloroplast according to a temperature. In addition, the temperature-controlled desiccator may be used to adjust a surrounding environment to 25° C. and 37° C. FIG. 29a is a graph showing an oxygen concentration (left) and AUC (right) in a hypoxic environment of chloroplast according to a temperature condition (torr-left, time*torr-right, n=24). FIG. 29b is a graph showing an oxygen concentration in a hypoxic environment of chloroplast according to a temperature condition (%, n=24).

FIG. 30 is a graph showing a result of measuring an oxygen concentration in a hypoxic environment of chloroplast according to a light (660 nm) condition. An experiment was performed in groups divided according to an intensity of light (660 nm) (FIG. 30a), a time of exposure to light (FIGS. 30b and 30c), and a persistence of the time of exposure to light (FIGS. 30d and 30e). After a hypoxic environment was induced using the temperature-controlled desiccator implemented in above FIG. 26, fresh isolated chloroplast at 37° C. diluted in media (RPMI+1% PS, 10% FBS) was prepared at 108/ml to put a chloroplast into a hypoxic environment (1% O2), and then an oxygen concentration of chloroplast solution was continuously measured using an oxygen plate reader for 20 hours, while varying light conditions. FIG. 30a is a graph showing a measured oxygen concentration in a hypoxic environment of chloroplast according to a light condition (torr-left, torr-right, n=24). FIG. 30b is a graph showing a measured oxygen concentration in a hypoxic environment of chloroplast according to a light condition (torr-left, torr-right, n=24). FIG. 30c is a graph showing a measured oxygen concentration in a hypoxic environment of chloroplast according to a light condition (torr-left, torr-right, n=24). FIG. 30d is a graph showing a measured oxygen concentration in a hypoxic environment of chloroplast according to a light condition (torr, n=24). FIG. 30e is a graph showing a measured oxygen concentration in a hypoxic environment of chloroplast according to a light condition (torr-left, torr-right, n=24).

FIG. 31 is a view showing an optical micrograph of alginate-CTP-chloroplast hydrogel with pancreatic cells encapsulated therein. FIG. 31a is a view showing that each group is classified. FIG. 31b is a view showing an optical micrograph of chloroplast-CTP (MFAFQYLLVM)-alginate hydrogel with pancreatic cells encapsulated therein. After chloroplast diluted in media (RPMI+10% FBS) was prepared according to each concentration, alginate-CTP solution diluted in distilled water and pancreatic cells extracted from an experimental animal (rat) were added by 20IEQ (*IEQ=pancreatic cell unit), respectively, put into 500 μl of 100 mM CaCl₂) solution, and reacted to prepare chloroplast-CTP-alginate hydrogel, and then it was confirmed using an optical microscope whether the pancreatic cells are well encapsulated in each group. As a result, it was confirmed that pancreatic cells are well encapsulated in hydrogel in all groups. FIG. 31c is a view showing a confirmed optical micrograph of chloroplast-CTP(MFAFQYLLVM)-alginate hydrogel in which pancreatic cells cultured in a hypoxic environment are encapsulated. After the above experiment, a hypoxic environment (1% O2) was created using the temperature-controlled desiccator and cultured for 24 hours, after which a degree of degradation of pancreatic cells in each group was confirmed using an optical microscope. As a result, it was confirmed that the morphology of the pancreatic cells is well maintained in each group.

FIG. 32 is a graph (%, n=5) quantitatively representing the viability of pancreatic cells encapsulated in alginate-CTP-chloroplast hydrogel at each concentration of chloroplast in a hypoxic environment. After the experiment of above FIG. 31, absorbance (OD450 nm) was measured for each group in two hours after dividing the CCK solution (10%) by 20 μl each, and then, as a result of correcting an absorbance value through an amount of DNA for each experimental group using Quant-iT™ PicoGreen™ dsDNA Assay Kits, it can be confirmed that viability of pancreatic cells may not be significantly decreased as compared to an IN group, except for an IH group, due to exposure to a hypoxic environment for a long time with regard to pancreatic cells in a hypoxic environment. It could be confirmed that viability of pancreatic cells is decreased about 18% in the IH group due to exposure to the hypoxic environment for 24 hours, representing that the hypoxic environment is not appropriate for culturing the pancreatic cells. It was confirmed that alginate-CTP-chloroplast (groups 4, 6) has a lower cell viability than that of the IN group, representing an insignificant result. With regard to alginate-CTP-chloroplast hydrogel, it means that the alginate-CTP-chloroplast material is not toxic to pancreatic cells regardless of a concentration of chloroplast. In addition, it was found that groups 0, 4, and 6 show higher cell viability than that of the IH group in a hypoxic environment, representing that pancreatic cells encapsulated in alginate-CTP-chloroplast hydrogel may have an appropriate condition for culturing in a hypoxic environment. (N.S: statistically no significant, *P≤0.05, **P≤0.01, ***P≤0.001, n=24. Results are shown as mean±S.E.M.)

FIG. 33 is a graph showing a result of static glucose-stimulated insulin secretion (GSIS) assay on pancreatic cells encapsulated in alginate-CTP-chloroplast hydrogel at each concentration of chloroplast in a hypoxic environment (insulin ng/DNA ng, n=5). After the experiment conducted in above FIG. 33, in two hours after dispensing a low glucose solution (2.8 mM) for each group, and in two hours after dispensing a high glucose solution (20.2 mM) for each group, an amount of insulin in each group was measured using a rat insulin ELISA assay kit. After that, an absorbance value was corrected through a DNA amount for each experimental group using Quant-iT™ PicoGreen™ dsDNA Assay Kits. It can be confirmed that an insulin secretion ability of pancreatic cells may not be significantly decreased compared to the IN group except for the IH group. It is confirmed that the IH group has a reduced insulin secretion ability in pancreatic cells due to exposure to a hypoxic environment for 24 hours. Alginate-CTP-chloroplast group shows a glucose stimulation index (SI) of pancreatic cells similar to that of the IN group, representing that insulin secretion was not reduced even though alginate-CTP-chloroplast group (groups 4, 6) was exposed to a hypoxic environment. In addition, it was found that groups 0, 4, and 6 show a significantly higher insulin secretion ability than that of the IH group in a hypoxic environment, representing that pancreatic cells encapsulated in alginate-CTP-chloroplast hydrogel did not have a reduced insulin secretion ability even though the pancreatic cells were cultured in a hypoxic environment, and may have an appropriate condition for culturing in a hypoxic environment.

FIG. 34 is a view showing a result of evaluating physical properties of an alginate-CTP-chloroplast microcapsule. The conditions for forming a microcapsule with an alginate-CTP-chloroplast conjugate using an encapsulator were optimized. In each group of 1) alginate, 2) alginate-chloroplast, and 3) alginate-CTP-chloroplast, a gelation ability of microcapsule was confirmed, and an alginate solution having a final concentration of 1.5% w/v alginate was mixed with chloroplast diluted in CIB (2.5×109/ml) at a ratio of 1:1 to prepare the microcapsules through an encapsulator, after which 300 μl of 100 mM CaCl₂) solution was added thereto and reacted to evaluate a gelation ability of each group. As a result, it was confirmed that microcapsules are well formed in all groups.

FIG. 35 is a view showing a result of evaluating a degree of degradation of alginate-CTP-chloroplast microcapsule.

FIG. 36 is a view showing a result of a rheology test using a rheometer to evaluate a mechanical strength of microcapsules for each group (kPa, n=2).

FIG. 37 is a view showing a result of evaluating an oxygen control ability of alginate-CTP-chloroplast microcapsule (μmol/L, n=6). In order to confirm an oxygen generation ability of CTP and chloroplast diluted in media (RPMI+10% FBS, 1% PS) and CIB, respectively, in the microcapsules, groups were divided into 1) free chloroplast (106, 107, 108/ml), 2) alginate-chloroplast (106, 107, 108/ml), and 3) alginate-CTP-chloroplast (106, 107, 108/ml) and an amount of oxygen generated was confirmed for 48 hours per group. (Alginate=1.5% (w/v)).

FIG. 38 is a view showing an optical micrograph of pancreatic cells for each group, encapsulated in an alginate-CTP-chloroplast microcapsule (4×, 10×, n=5). In order to evaluate the toxicity of the alginate-CTP-chloroplast microcapsule and confirm the insulin secretion ability of the encapsulated pancreatic cells, the pancreatic cells (islet) extracted from an experiment animal (fat) were encapsulated in the microcapsule using an encapsulator. An experiment was performed in groups divided into each of 0) Intact Islet (Normoxia) (*Normoxia: normoxic environment), 1) Intact Islet (Hypoxia) (*Hypoxia: hypoxic environment), 2) Alginate+Islet, 3) Alginate-Chloroplast+Islet, and 4) Alginate-CTP-Chloroplast+Islet, and then a solution was prepared by mixing an alginate solution having a final concentration of alginate at 1.5% and chloroplast (2.5×109/ml) diluted in CIB at a ratio of 1:1, after which a microcapsule was prepared with the addition of 500IEQ (*IEQ=pancreatic cell unit) of pancreatic cells extracted for each group by using an encapsulator.

FIG. 39 is a view showing a result of performing CCK-8 Assay to evaluate the viability and necrosis of pancreatic cells for each group, encapsulated in alginate-CTP-chloroplast microcapsules (%, n=5). For each group of the above experiment, 1 ml of a CCK solution (10%) was dispensed, and then absorbance (OD450 nm) was measured in two hours later. After that, this is a result of correcting an absorbance value through an amount of DNA for each experimental group using a Quant-iT™ PicoGreen™ dsDNA Assay Kits. Due to exposure of pancreatic cells to a hypoxic environment for a long time, viability of pancreatic cells was significantly reduced to about 22% compared to pancreatic cells in a normoxic environment, representing that a hypoxic environment is not an environment appropriate for pancreatic cells to survive. As a result of confirming viability of pancreatic cells encapsulated in alginate-CTP-chloroplast microcapsules, the cell viability was reduced by about 10% compared to pancreatic cells in a normoxic environment, representing that the alginate-CTP-chloroplast material may help the survival of pancreatic cells. When comparing an alginate-chloroplast microcapsule with an alginate microcapsule, it was confirmed that the chloroplast of the alginate-chloroplast microcapsule may produce oxygen which may help the survival of the cells. When comparing an alginate-CTP-chloroplast microcapsule with an alginate-chloroplast microcapsule, the reduction of cell viability was lowered in the alginate-CTP-chloroplast microcapsule, representing that the efficacy of CTP plays an important role in the production of oxygen by chloroplast, and thus that CTP-stabilized chloroplast has a more advantage in terms of functions. When comparing the cell viability for each group, the cell viability of the pancreatic cells encapsulated in the alginate-CTP-chloroplast microcapsule was lower than that of the pancreatic cells in the normoxic environment, but this is probably affected by metabolites (metabolome) or reactive oxygen (ROS) of the pancreatic cells present in the alginate-CTP-chloroplast microcapsule. Accordingly, in order to compensate for this problem in subsequent experiments, an experiment was performed by synthesizing catalase (CAT) or superoxide dismutase (SOD) enzyme.

FIG. 40 is a fluorescent micrograph showing a result of performing a live & dead assay in order to confirm the viability of pancreatic cells for each group after being exposed to a hypoxic environment (1% O2) for 24 hours after being encapsulated in alginate-CTP-chloroplast microcapsules with pancreatic cells encapsulated therein (4×10×, n=5, green fluorescent-live cells, red fluorescent-dead cells). In order to evaluate the cellular toxicity for the hypoxic environment and the microcapsule environment in each group of the above experiment, microcapsules were prepared (group 0: Intact Islet (Normoxia) excluded), and then exposed to light (660 nm, 1000 lux) twice for initial 150 minutes at a time interval of 30 minutes so that the efficacy of chloroplast according to the influence of light may be confirmed, and then subjected to live & dead assay. For each group, in 40 minutes after dispensing 1 ml of a solution in which 5 μl of calcein and 20 μl of EthD were mixed in PBS, the viability of pancreatic cells was confirmed using a fluorescence microscope. As a result, it was confirmed that a green signal appears to be relatively strong in the alginate-CTP-chloroplast microcapsule group compared to other groups in a hypoxic environment, and a yellow signal appears to be the weakest as a result of the combination of green and red. This means that the alginate-CTP-chloroplast microcapsule is an environment appropriate for pancreatic cells to survive, as in the CCK-8 assay result of above FIG. 39.

FIG. 41 is a graph showing a result of performing a glucose-stimulated insulin secretion (GSIS) assay to evaluate the function of pancreatic cells encapsulated in the microcapsule of each group through an insulin secretion ability as a result of encapsulating alginate-CTP-chloroplast microcapsules with pancreatic cells encapsulated therein (insulin ng/DNA ng, n=5).

FIG. 42 is a view showing a sequence of chloroplast-transit-peptide (CTP, VILGLGLAGI, hereinafter CTP(V-)) peptide which may be inserted into an additionally set chloroplast outer membrane (VILGLGLAGI, 0.924 kDa). The VILGLGLAGI peptide was synthesized to obtain a peptide having a purity of 95%, which was confirmed as 0.924 kDa. In addition, it was confirmed that the VILGLGLAGI peptide has a hydrophilicity of 0%, unlike the MFAFQYLLVM peptide.

FIG. 43(A) is a view showing confocal microscopic and optical microscopic images for binding CTP(V-)-FITC(green fluorescence) to a surface of chloroplast (red fluorescence). FIG. 43(B) is a view showing an image of the presence of FITC-VILGLGLAGI peptide physically bound to a surface of chloroplast confirmed by an optical microscope. CTP(V-)-FITC with CTP(V-) and chloroplast was treated and analyzed by a confocal microscope and an optical microscope for each time (1, 5, 10, 30, 60 min).

FIG. 44 is a schematic view showing a chemical binding reaction formula between COOH of alginate and N-terminus moiety of CTP(V-) using the EDC/NHS method. A carbodiimide coupling reaction was used to form an amide bond with carboxylate (—COOH) group of alginate and primary amine of CTP(V-) to prepare a conjugate. EDC/NHS was used to increase the efficiency of the carbodiimide coupling reaction.

FIG. 45 is a table showing a conjugate synthesis ratio performed at various concentrations in order to confirm the most optimal ratio of alginate-chloroplast-transit-peptide (CTP(V-)) conjugate. For the synthesis of CTP(V-)-alginate, sodium alginate (0.1 mg/ml, 0.5 mg/ml, 1 mg/ml) was dissolved in 10 ml of 0.1 M MES buffer at pH 6 at each concentration, and then 2 mM EDC and 5 mM NHS were added thereto, and reacted at room temperature for 15 minutes. After titration to pH 7, 10 mg/ml of peptide dissolved in 1% DMSO was added thereto, and reacted at room temperature for two hours. After performing dialysis, the resulting product was freeze-dried to obtain a composite.

FIG. 46 is a view showing an FT-IR result for confirming the synthesis results according to a concentration of the alginate-chloroplast-transit-peptide (CTP(V-), LDY004) conjugate.

FIG. 47 is a view showing a ¹H-NMR result for confirming the synthesis results according to a concentration of the alginate-chloroplast-transit-peptide (CTP(V-), LDY004) conjugate.

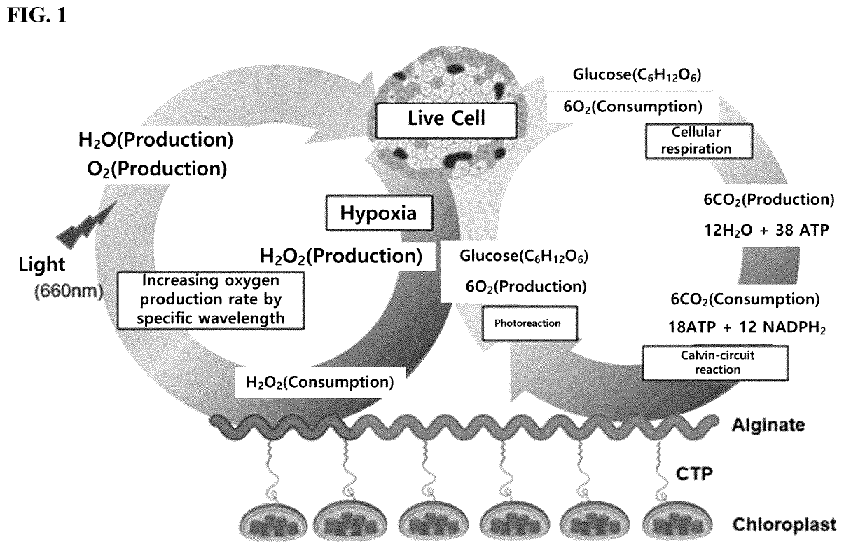

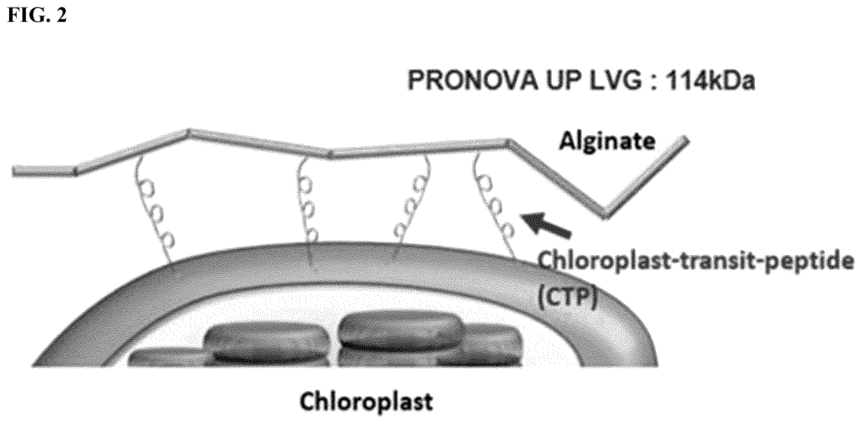

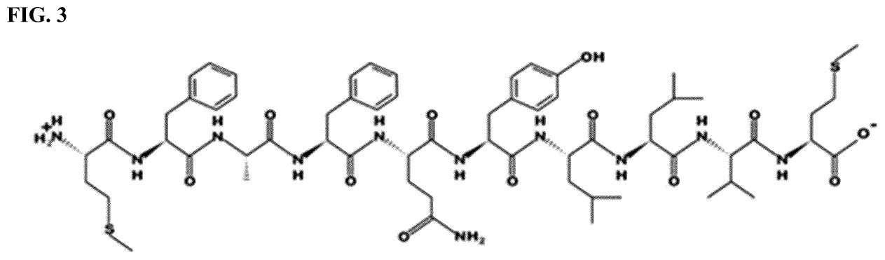

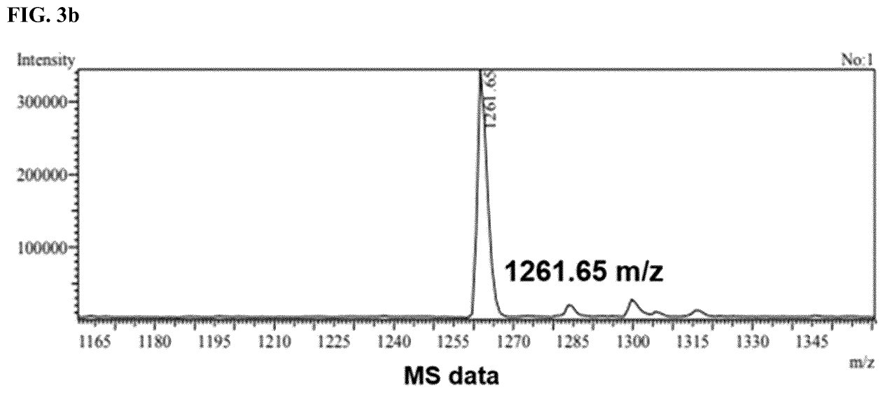

### MODE FOR INVENTION

Hereinafter, the present invention will be described in more detail through exemplary embodiments. However, these exemplary embodiments are provided only for the purpose of illustrating the present invention, and thus the scope of the present invention is not limited thereto.

### EXAMPLE

### Example 1. Alginate-CTP Conjugation

Alginate (—COOH) and CTP (—NH2) were conjugated by the following method to obtain a composite.

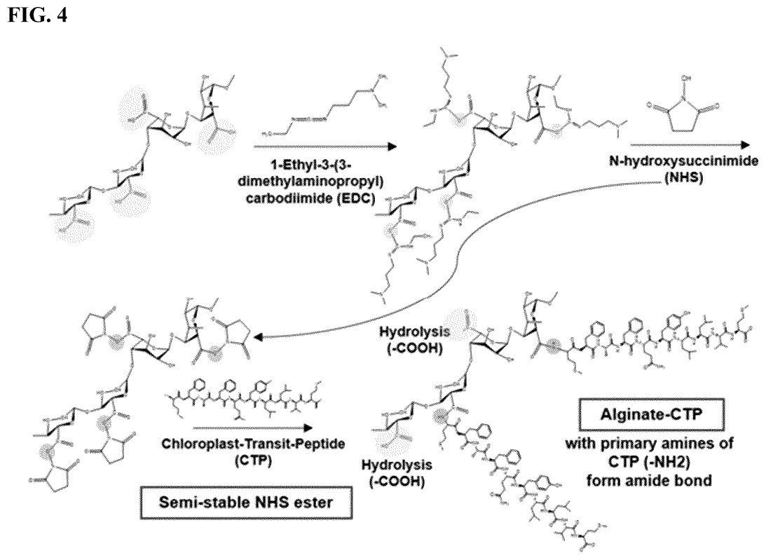

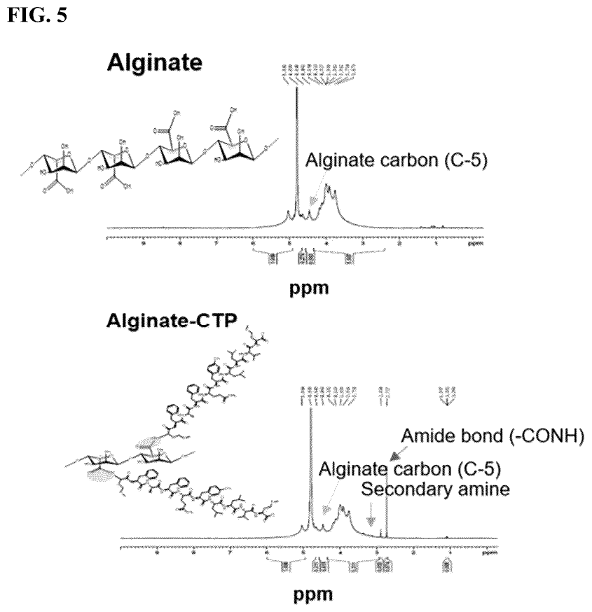

After preparing 10 ml of 0.1 M MES buffer (+0.5 M NaCl), pH was adjusted to 6. 0.2 mM sodium alginate (228 mg/10 ml) was dissolved in 10 ml of MES buffer prepared above, so that sodium alginate might be well loosen and might not be agglomerated. When solution was sufficiently dissolved, 4 mg/10 ml of EDC (191.7 MW) corresponding to 2 mM was dissolved. After that, 6 mg/10 ml of NHS (5 mM) was added thereto and reacted at RT for 15 minutes. The pH was raised to 7 using PBS or sodium bicarbonate (NaHCO₃, sodium hydrogen carbonate). Peptide (10 mg/ml, peptide was synthesized upon request by Peptron, a peptide synthesis company) dissolved in 1% DMSO was added to solution in an amount corresponding to a molar ratio (1:1, 1:5, 1:10, 1:25, 1:50). After that, the resulting mixture was reacted at RT for two hours. The resulting mixture was subjected to dialysis for three days using a molecular weight cut-off (MWCO) dialysis membrane of 6,000 to 8,000 g/mol. The resulting product was frozen in a deep freezer, and then freeze-dried to obtain an alginate-CTP composite in the form of powder (see FIG. 4).

### Example 2. BCA Assay

An amount of CTP (protein) in an alginate-CTP composite was quantified by the following method.

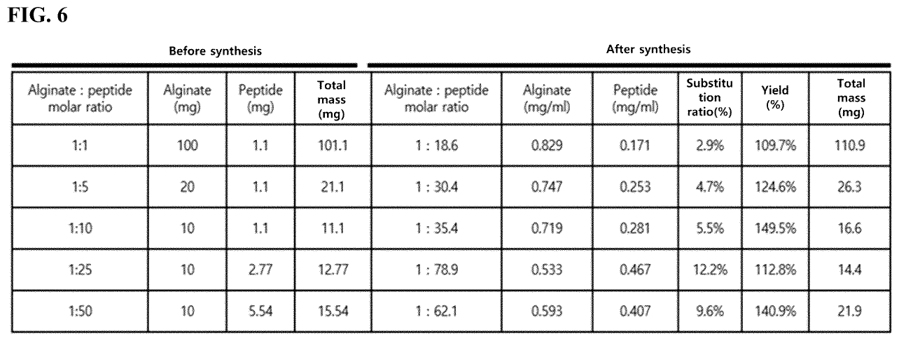

Controls A to I were prepared by using a BCA assay kit. Foil was spread on ice and placed at 96-well plate, after which 24 μl of control was loaded thereon. A sample was loaded under a line with the control loaded thereon. (n=5, concentration of 2 mg/ml (RIPA solution)) After that, 200 μl of A+B solution per well was loaded on the loaded sample. At this time, solutions A and B need to be kept at room temperature (A=196 μl, B=4 μl, A:B=50:1). The control was added starting from a low concentration. After incubation for 30 minutes, an absorbance was measured at 562 nm using an instrument (see FIG. 6).

As a result, a moral ratio of 1:19, 1:31, 1:36, 1:79 and 1:62 could be confirmed in order. Thus, it was confirmed that the synthesis is well performed, as the molar ratio value becomes less (1:1, 1:5).

### Example 3. Alginate-CTP Gelation and Rheology Test

A strength of hydrogel formed for each molar ratio of the alginate-CTP composite was confirmed by the following method.
Group (n=5)
(1) Alginate (control)
(2) Alginate-CTP (1:1)
(3) Alginate-CTP (1:5)
(4) Alginate-CTP (1:10)
(5) Alginate-CTP (1:25)
(6) Alginate-CTP (1:50)

To prepare an alginate or alginate-CTP solution (0.07 mM), 0.9% NaCl,

1.5% alginate or alginate-CTP were added and diluted with tertiary distilled water (500 μl of tertiary distilled water+4.5 mg of NaCl+4 mg of alginate or alginate-CTP). At this time, alginate powder was slowly added and sufficiently diluted. In order to prepare a CaCl2) solution (100 mM), 11 g of CaCl2) (110.98 g/mol) and 2.6 g of HEPES were added to 1 L of tertiary distilled water. The prepared CaCl2) solution was filtered and refrigerated. After that, 200 μl of the solution of process 1 and 500 μl of 100 mM CaCl2) solution were reacted in 48-well plate for 30 minutes to carry out gelation. The strength of hydrogel was confirmed by measuring rheology through a rheometer.

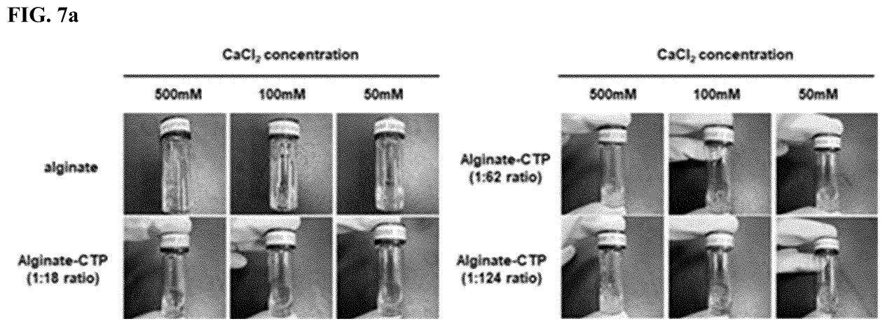

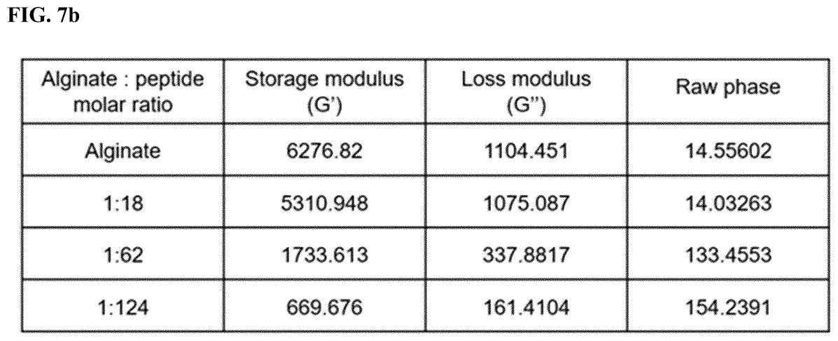

As a result, it was confirmed that capsule gel is not formed at an alginate-CTP feed molar ratio of 1:280 or more (see FIG. 7a). This means that too many peptides prevent gel formation by calcium. All groups showed better gel formation as well as a solid appearance, as a molarity of the calcium solution becomes higher.

In addition, as a result of a rheology test on the prepared capsule gel, it was found that alginate shows 6 kPa (G′) based on 100 mM CaCl₂), which is decreased as the feed molar ratio increases, thus obtaining a value of 5.3 kPa(G′) at a molar ratio of 1:18 and a value of 1.7 kPa(G′) at a molar ratio of 1:62, and it could be confirmed that G′(storage modulus) values are greater than G′(loss modulus) in all groups (see FIG. 7b). Thus, it may be determined that the capsule gel is formed. On contrary, it was confirmed that the capsule gel is not formed in less than 1 kPa(G′) from a higher molar ratio of 1:128.

### Example 4. Chloroplast Isolation

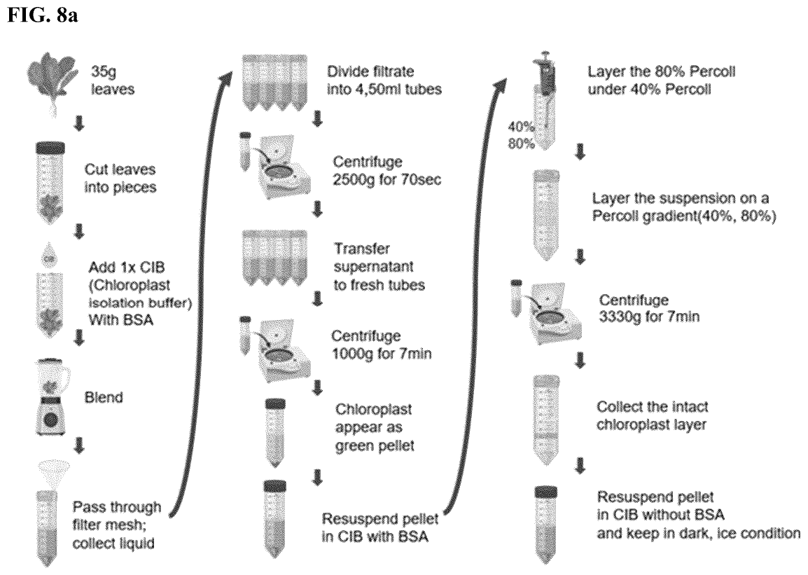

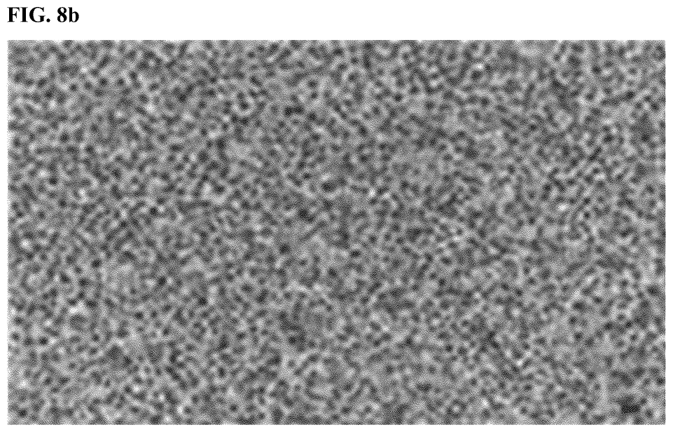

Chloroplast was isolated from spinach by the following method (see FIG. 8).

The materials included 35 g of spinach leaves, scissors, a blender, gauze, a conical tube, cooling centrifuge, 1× chloroplast isolation buffer (CIB) without BSA (0.33 M sorbitol, 0.1 M tris-cl pH 7.8, 5 mM MgCl2, 10 mM NaCl, 2 mM EDTA), 1× chloroplast isolation buffer with BSA (0.1% W/V), 40% percoll (4 ml percoll and 6 ml 1×CIB buffer with BSA) and 80% percoll (8 ml percoll and 2 ml 1×CIB buffer with BSA).

All experiments were rapidly performed at a cold room or under a cold condition. 35 g of spinach leaves, excluding the leaf vein, were obtained, and the spinach leaves were cut into small pieces having a width of 1 cm using scissors which were sterilized with ethanol. 120 ml of CIB buffer containing BSA was placed in a cold blender, and blending was performed 10 times for two seconds along with spinach leaves. The above solution was poured into a beaker covered with several layers of gauze having ice therein and was subjected to filtration. The filtered solution was placed in a conical tube and subjected to centrifugation at 4° C., 2500 RPM for 70 seconds. The supernatant was transferred to a new conical tube, and then subjected to centrifugation at 4° C., 2500 RPM for seven minutes. The supernatant was discarded and a green pellet was gently loosen with finger tapping. The pellet was resuspended with 2 ml of 1×CIB buffer with BSA and was subjected to pipetting. 40%, 80% percoll solution was prepared, and 3 ml of 80% percoll, 3 ml of 40% percoll, and 6 ml of chloroplast suspension were carefully placed in 15 ml conical tube in order. The reaction mixture was subjected to centrifugation at 4000 RPM for 10 minutes. Only the solution between the 80%/40% percoll layers containing intact chloroplast was carefully taken and transferred to another conical tube. After that, the obtained pellet was mixed with 10 ml of 1×CIB buffer without BSA, and then wrapped in foil and stored in a refrigerated state.

### Example 5. Evaluation of DMSO Toxicity to Chloroplast

It was evaluated whether or not DMSO (10 mg/ml) used to dissolve peptide was toxic to chloroplast (1*108/ml) for each time by the following method.

0% and 1% DMSO was prepared, diluted in 1 ml of 1*108/ml chloroplast in CIB solution, and treated for 0, 5, 10, 30 and 60 minutes. 10 μl of the solution was taken for each time, and the number of chloroplasts was counted using a hemocytometer.

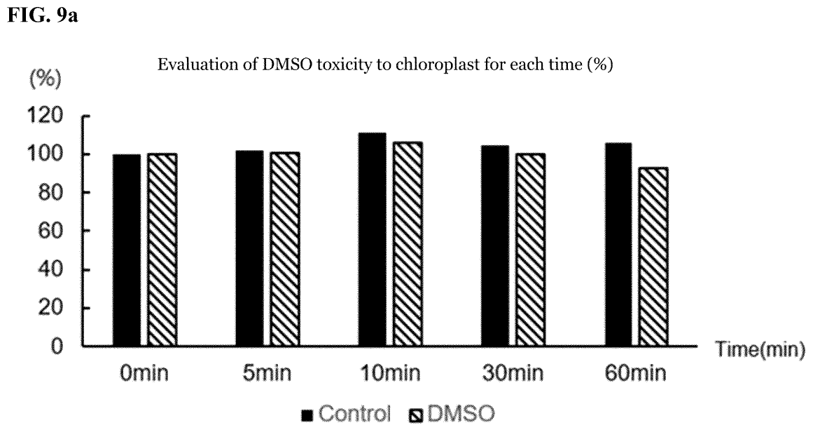

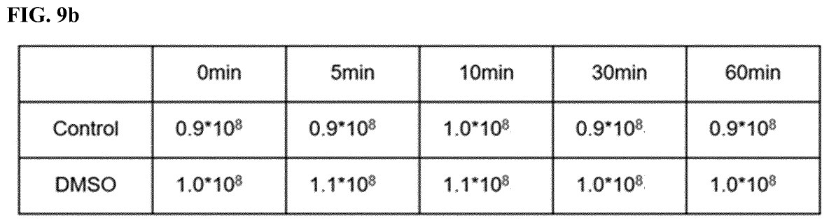

As a result, there was no significant difference between a control group not treated with DMSO and a group treated with 1% DMSO, and it was confirmed that 1% DMSO does not affect chloroplast (see FIG. 9).

### Example 6. Measurement of Chlorophyll of Chloroplast

Chlorophyll was measured to quantify chloroplast by the following method.

Using CIB, 2*108/ml chloroplasts were diluted from 0% to 100% with a total of 14 sections. 250 μl of 80% acetone (+5 mg/ml pH 7 EDTA) was added to 250 μl of each diluted solution. 80% acetone (+5 mg/ml pH 7 EDTA) was performed to obtain chlorophyll by crushing the chloroplast, so as to obtain a solution having a final concentration of 1*108/ml (based on 100%). UV-VIS absorbance at 645 nm, 663 nm was measured using a nanodropdrop. After that, a μg/ml value of total chlorophyll was obtained by substituting the measured value into the following Arnons equation.

<Arnons Equation>

*Chlorophyll a(μg/ml)=12.7(A663)−2.69(A645)

*Chlorophyll b(μg/ml)=22.9(A645)−4.68(A663)

*Total chlorophyll (μg/ml)=20.2(A645)+8.02(A663)

### Example 7. Confirmation of Binding of Chloroplast Outer Membrane to CTP-FITC

7.1. Confirmation of Binding of Chloroplast Outer Membrane to CTP-FITC Using Fluorescent Image

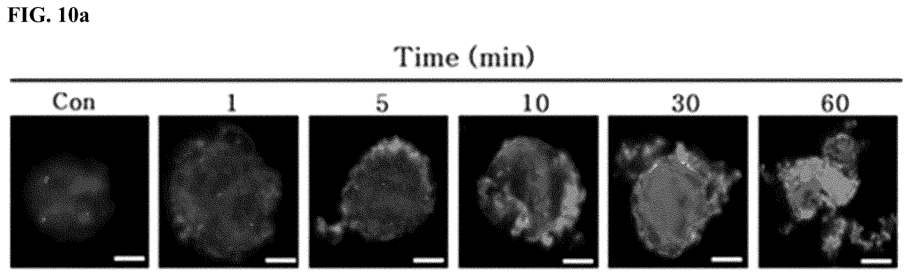

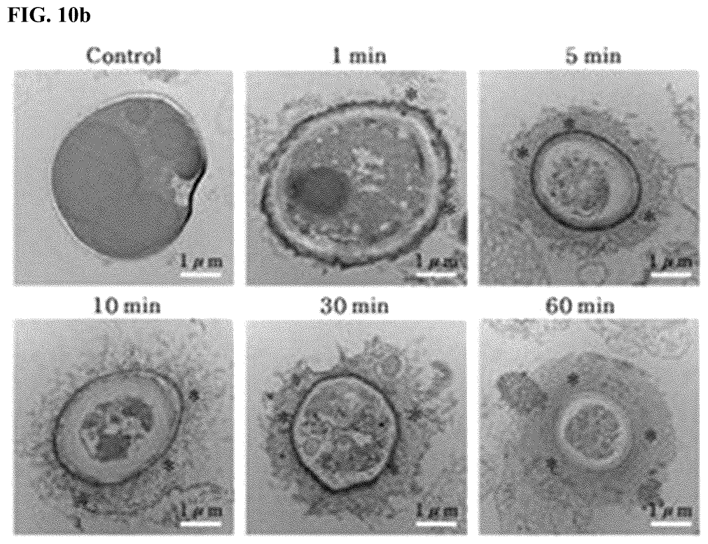

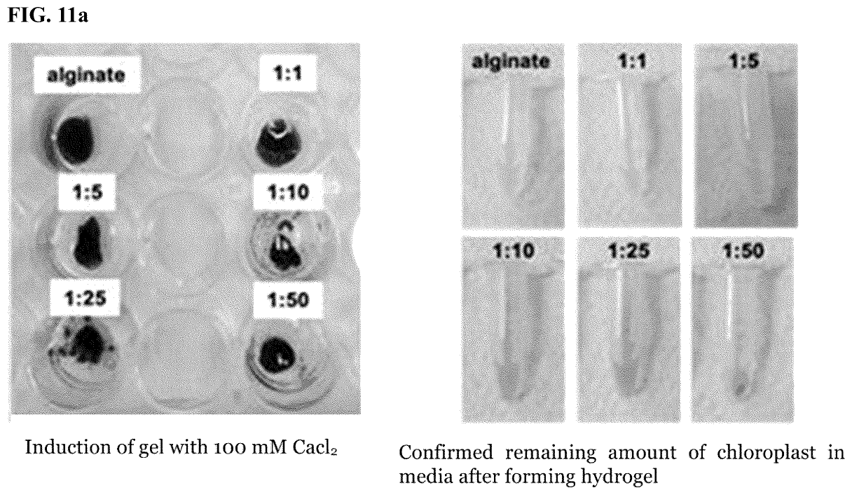

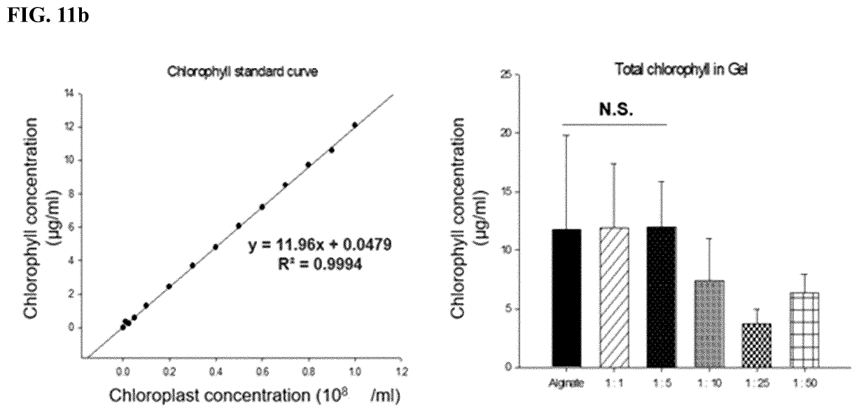

It was confirmed by fluorescence that peptide-FITC may be anchored on an outer membrane of chloroplast by the following method (see FIG. 10a).

Care was taken not to be exposed to light during the experiment. 500 μl of 1*108/ml chloroplast (control) and 500 μl of 1*108/ml chloroplast+CTP-FITC were prepared. At this time, CTP-FITC was obtained upon request from Anygen, a synthesis company. In 1*108/ml chloroplast+CTP-FITC, CTP-FITC was treated at a concentration of 0.6 mg/ml. At this time, if 500 μl of 1*108/ml chloroplast (in CIB) is taken and 30 μl of 10 mg/ml CTP (in DMSO) is taken and added, the final concentration may become 0.6 mg/ml. After that, 100 μl was taken and immobilized every 1, 5, 10, 30 and 60 minutes, and the solution reacted for each time was subjected to centrifugation at 13500 RPM for one minute. A pellet was obtained, immobilized in a 2.5% glutaraldehyde (0.05 M SCB) fixative (500 μl, 15 min), and washed twice with PBS to remove unreacted CTP-FITC. After that, 500 μl was filled with CIB buffer, and then mounted using slide glass/cover glass. A fluorescent photograph was taken using a confocal microscope, and sectioning was performed with the thickness of about 1 μm (green fluorescence (FITC)=488 nm, red fluorescence (chloroplast auto fluorescence)=580 nm).

7.2. Confirmation of Binding of Chloroplast Outer Membrane to CTP-FITC Using TEM

It was confirmed by TEM whether peptide-FITC may be anchored on an outer membrane of chloroplast by the following method (see FIG. 10b).

500 μl of 1*108/ml chloroplast (control) and 500 μl of 1*108/ml chloroplast+CTP-FITC, which were reacted by the same method and at the same time (1, 5, 10, 30 and 60 minutes) as in above 7.1, were immobilized in 1 ml of 2% glutaraldehyde+2% paraformaldehyde in 0.05 M sodium cacodylate buffer fixative for 15 minutes. After immobilization, in order to remove the unreacted CTP-FITC, PBS washing was repeated twice to obtain a pellet. 1 ml of 2% glutaraldehyde+2% paraformaldehyde in 0.05 M sodium cacodylate buffer was added to the obtained pellet, and reacted at 4° C. overnight. After a TEM pre-treatment process (prepared in a block with resin after immobilizing chloroplast) was performed, fine flakes were produced using an ultramicrotome, TEM was photographed and confirmed, and the TEM pre-treatment and subsequent processes were conducted at the Joint Research Institute of Seoul National University's College of Agriculture and Life.

7.3. Sub-Conclusion

As a result of the above experiment, it could be confirmed that the CTP is sufficiently coated by anchoring on an outer membrane of chloroplast starting from a reaction time of five minutes or more, and then the reaction time of the CTP and the chloroplast was set to five minutes.

### Example 8. Hydrogel Gelation and Rheology Test

8.1. Alginate-CTP Hydrogel Gelation and Rheology Test (CIB)

A degree of gelation of alginate-CTP and CaCl₂) solution in the CIB was confirmed by the following method to determine whether the use of the CIB has no effect when the alginate-CTP and chloroplast is anchored. At this time, it was assumed that gelation may not occur.
Group (n=5)
(1) Alginate (control)
(2) Alginate-CTP (1:50)

Alginate (control) and alginate-CTP (1:50) were prepared, and a 100 mM CaCl₂) solution, which was a positive control for confirming the existing gelation function, and Ca2+, Mg2+-free PBS, which was a negative control, and the CIB, which was an experimental group where gelation was not expected were prepared. 4 mg of alginate or alginate-CTP, 4.5 mg of NaCl, and 500 μl of tertiary distilled water were well mixed. 250 μl of the solution of process 3 was reacted with the CIB in 48-well plate for 30 minutes. The strength of gel was confirmed (about having the properties of gel) by measuring rheology through a rheometer.

8.2. Alginate-CTP-Chloroplast Hydrogel Gelation and Rheology Test

The following method was performed to determine whether gelation occurs by reacting the alginate-CTP composite with chloroplast, and to measure the strength of the hydrogel thus produced.
Group (n=5)
(1) Alginate (control)
(2) Alginate-CTP (1:1)
(3) Alginate-CTP (1:5)
(4) Alginate-CTP (1:10)
(5) Alginate-CTP (1:25)
(6) Alginate-CTP (1:50)

Alginate (control) and alginate-CTP (a molar ratio of 1:1, 1:5, 1:10, 1:25 and 1:50) were prepared, and 4 mg of alginate or alginate-CTP, 4.5 mg of NaCl, and 500 μl of tertiary distilled water were well mixed. Chloroplast isolated from spinach on the same day was prepared at a concentration of 1*108/ml. 250 μl of the solution of process 2 and 250 μl of the solution of process 3 were well mixed in 48-well plate, and then reacted with 100 mM CaCl2) for 30 minutes. The strength of hydrogel was confirmed (about having the properties of hydrogel) by measuring rheology through a rheometer.

### Example 9. Chloroplast Quantification

9.1. Quantification of Chloroplast Using Residual Solution of Alginate-CTP-Chloroplast Hydrogel

After the removal of alginate-CTP-chloroplast hydrogel, a residual solution was used to quantify chloroplast in hydrogel by the following method.

Alginate-CTP-chloroplast hydrogel was prepared as in the method of above 9.2, and all solutions excluding hydrogel were recovered and subjected to centrifugation at 13,500 RPM for one minute. A pellet was dissolved in 250 μl of 80% acetone after being suspended (repeated three times of vortexing & stay). After measuring absorbance at 663 nm and 645 nm using a nanodropdrop, the measured value was used to calculate an amount of total chlorophyll (μg/ml) according to the following Arnons equation.

<Arnons Equation>

*Chlorophyll a(μg/ml)=12.7(A663)−2.69(A645)

*Chlorophyll b(μg/ml)=22.9(A645)−4.68(A663)

*Total chlorophyll (μg/ml)=20.2(A645)+8.02(A663))

9.2. Quantification of Chloroplast Present in Alginate-CTP-Chloroplast Hydrogel

The chloroplast contained in the alginate-CTP-chloroplast hydrogel was quantified by the following method.

EDTA was added at 5 mg/ml to PBS, and the pH was increased to 7. An alginate lyase was placed in an EDTA solution, and the vortexing & stay was repeated three times.

When hydrogel was loosen, centrifugation was performed at 13500 RPM for one minute, and a pellet was suspended in 250 μl of 80% acetone. After that, vortexing & stay was repeated three times to perform dissolution, and absorbance was measured at 663 nm and 645 nm using a nanodrop drop. The measured absorbance was substituted into Arnons equation below to calculate an amount of total chlorophyll (μg/ml).

<Arnons Equation>

Chlorophyll a(μg/ml)=12.7(A663)−2.69(A645)

Chlorophyll b(μg/ml)=22.9(A645)−4.68(A663)

Total chlorophyll (μg/ml)=20.2(A645)+8.02(A663)

9.3. Sub-Conclusion

As a result of the above experiment, it was confirmed that the hydrogel efficiency of alginate-CTP having a feed molar ratio of 1:1 and 1:5 is very good with an encapsulation rate of about 99%. Thus, it was confirmed that alginate-CTP having a feed molar ratio of 1:1 and 1:5 may effectively bind to almost all chloroplasts (see FIG. 11).

### Example 10. Method for Confirming Alginate-CTP-Chloroplast Hydrogel Gelation for Each Time

It was confirmed whether the alginate-CTP-chloroplast hydrogel is loosen over time by the following method.

An alginate-CTP-chloroplast hydrogel was prepared according to the method of above Example 9. After that, a rheology test was performed through a rheometer to confirm whether hydrogel is loosen for four weeks to confirm the strength of hydrogel.

### Example 11. Evaluation of Toxicity of Chloroplast to HEX 293 T Cell (CCK-8 Assay)

In order to determine whether chloroplast affects the cells, CCK-8 assay was performed using HEK 293 cells, which are a general cell line, so as to confirm the results.
Group (n=5, diluted with CIB).
(1) Control (HEK 293 T cell+DMEM media)
(2) Chloroplast 1*102/ml
(3) Chloroplast 1*104/ml
(4) Chloroplast 1*106/ml
(5) Chloroplast 1*108/ml
(6) CIB

HEK 293 T cells (3.5*10⁴/well) stabilized at 96-well plate for 24 hours were prepared (n=5). Each of the prepared groups was treated with 100 μl of the prepared chloroplast solution at each concentration. After incubation for 24 hours, the chloroplast solution was suctioned. The resulting product was washed twice with PBS and treated with 100 μl of media (DMEM)+10 μl of CCK solution. After incubation by wrapping the foil for two hours, absorbance was measured at 450 nm using a reader. At this time, 50 μl of only the supernatant was taken to reduce the effect of chloroplast absorbance.

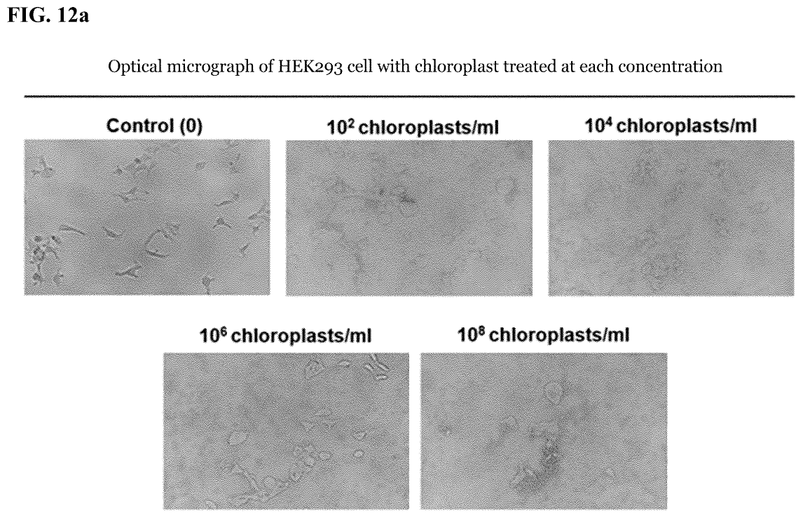

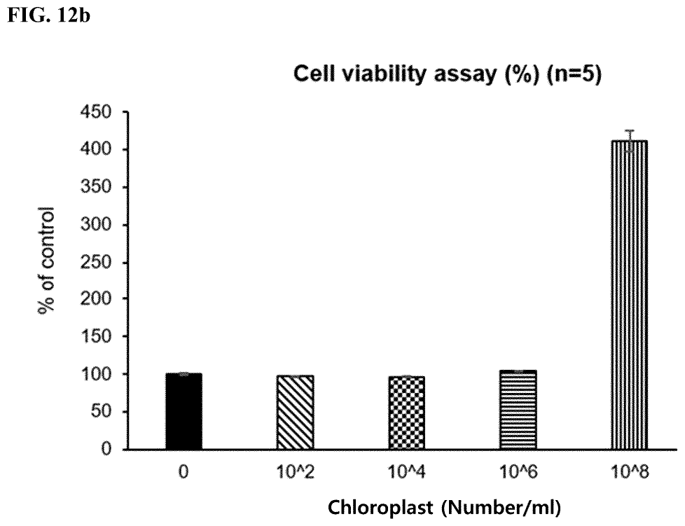

As a result, even if the concentration of chloroplast is increased, it was confirmed that there is no toxicity to the cells (see FIG. 12). In the case of chloroplast at a concentration of 108/ml, it is concluded that the absorbance is high by chloroplast.

### Example 12. Measurement of Amount of Oxygen Generated from Chloroplast

12.1. Measurement of Amount of Oxygen Generated from Chloroplast Depending on Presence or Absence of Light

An amount of oxygen generated from chloroplast depending on the presence or absence of light was confirmed by the following method.
Group (n=5, diluted with CIB).
(1) Control (CIB)
(2) Chloroplast 1*102/ml
(3) Chloroplast 1*104/ml
(4) Chloroplast 1*106/ml
(5) Chloroplast 1*108/ml

The chloroplast was diluted in 96-well plate in accordance with the group by using the CIB, and 300 μl was added thereto. An amount of oxygen generated from chloroplast depending on the presence or absence of light (660 nm) was measured using an oxygen sensor. At this time, OxyLite (Oxford Optronix, U.K.) was used as the oxygen sensor. A distance between material and light was set to 5 cm. An amount of oxygen generated was measured in 0, 30, 60 and 120 min units using the oxygen sensor (reading time: 3 min).

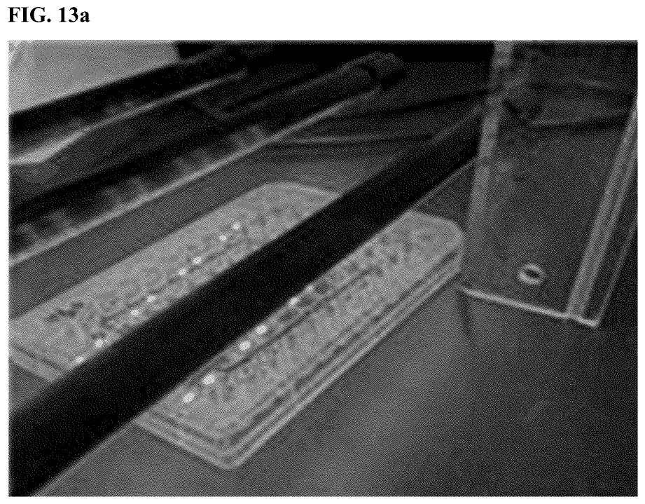

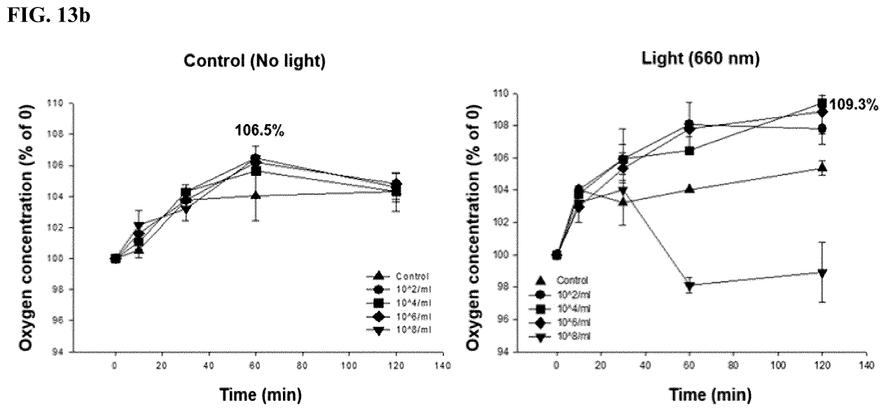

As a result, the group (no light) not exposed to light (660 nm) showed a level of oxygen generation up to 6.5% higher, but the control group showed the same level of oxygen generation as that of visible light under the light (660 nm) condition, whereas the group having chloroplast inserted showed a level of oxygen generated up to 9.3% higher. It was confirmed that oxygen generation is rather reduced at a high concentration of chloroplast than that of the control group (see FIG. 13).

12.2. Measurement of Amount of Oxygen Generated from Alginate-CTP-Chloroplast Hydrogel Depending on Presence or Absence of Light

An amount of oxygen generated from alginate-CTP-chloroplast hydrogel depending on the presence or absence of light was confirmed by the following method.
Group (n=5, diluted with CIB, and immobilized with a molar ratio of alginate:peptide=1:5).
(1) Control (CIB)
(2) Chloroplast 1*102/ml
(3) Chloroplast 1*104/ml
(4) Chloroplast 1*106/ml
(5) Chloroplast 1*108/ml

An alginate-CTP-chloroplast hydrogel was prepared by adding chloroplast to 96-well plate in accordance with the group. An amount of oxygen generated from chloroplast depending on the presence or absence of light (660 nm) was measured using an oxygen sensor. At this time, OxyLite (Oxford Optronix, U.K.) was used as the oxygen sensor. A distance between material and light was set to 5 cm. An amount of oxygen generated was measured in 0, 30, 60 and 120 min units using the oxygen sensor (reading time: 3 min).

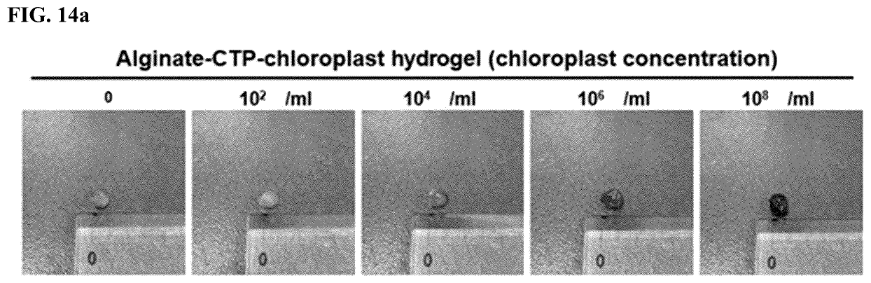

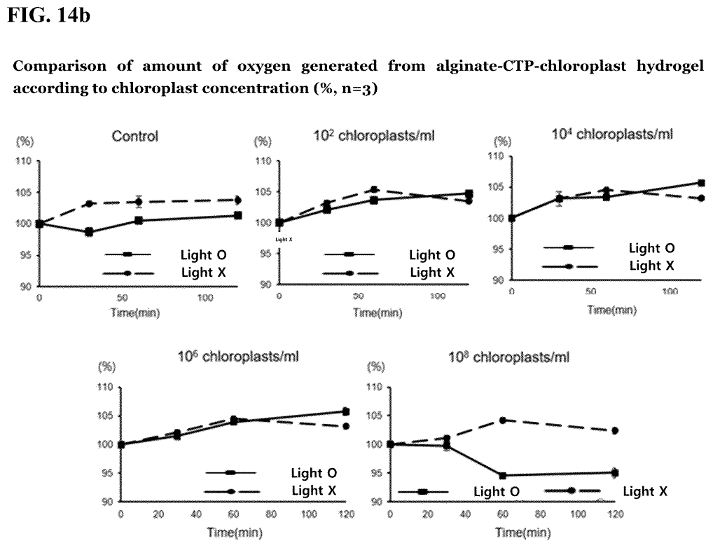

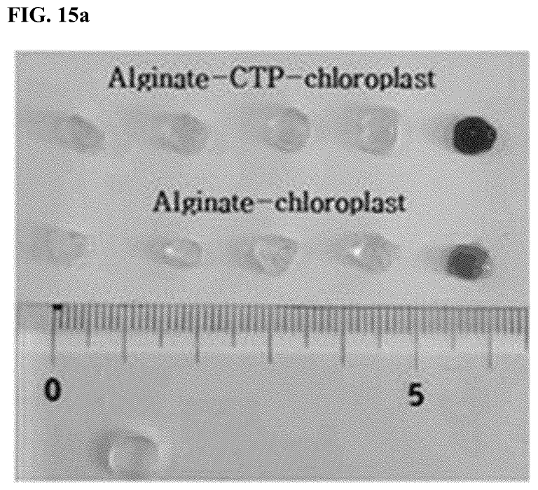

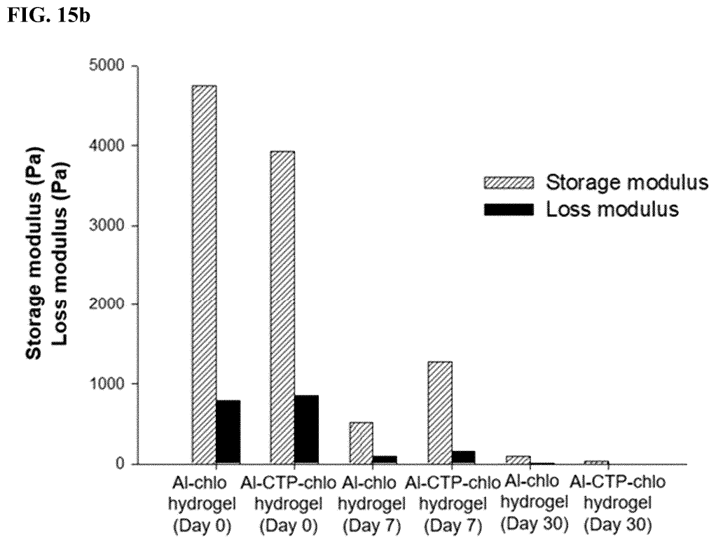

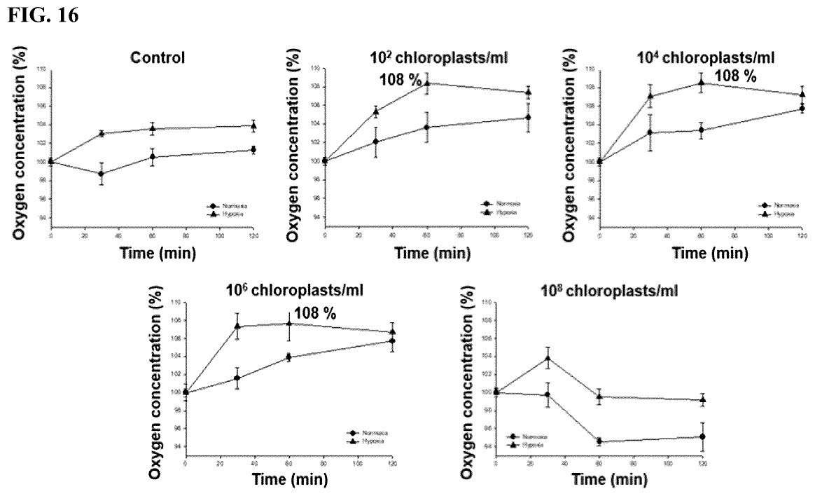

As a result, it was confirmed that an amount of oxygen generated under a partial pressure increases to about 105% as time passed, and an amount of oxygen generated increases under the light (660 nm) condition. On contrary, when not exposed to light (660 nm), an amount of oxygen generated appeared to increase in the beginning, but appeared to decrease in 120 minutes later. It was confirmed that hydrogel containing 108/ml of chloroplast shows a decrease up to about 95% under the light (660 nm) condition (see FIG. 14b).

It represents an increase compared to under the condition for measuring an oxygen concentration of chloroplast per se, not hydrogel (about 55%).

### Example 13. Measurement of Amount of Oxygen Generated in Normoxic and Hypoxic States

13.1. Measurement of Amount of Oxygen Generated from Alginate-CTP-Chloroplast Hydrogel in Normoxic and Hypoxic States

An amount of oxygen generated from alginate-CTP-chloroplast hydrogel according to exposure to light in normoxic and hypoxic states was confirmed by the following method.
Group (n=5, diluted with CIB, and a synthesis ratio was fixed at a molar ratio of alginate:peptide=1:5.)
(1) Control (CIB)
(2) Chloroplast 1*102/ml
(3) Chloroplast 1*104/ml
(4) Chloroplast 1*106/ml
(5) Chloroplast 1*108/ml

An alginate-CTP-chloroplast hydrogel was prepared by adding chloroplast to 96-well plate in accordance with the group. The 96-well plate was implemented in normoxic and hypoxic states under the following conditions.
Normoxic state: To implement a typical 21% oxygen environment
Hypoxic state: To implement a 1% oxygen environment by injecting a mixed gas (94% N2+5% CO2+1% O2) into a hypoxia chamber experiment instrument

After that, an amount of oxygen generated from chloroplast according to light exposure in the normoxic and hypoxic states was measured using an oxygen sensor. At this time, OxyLite (Oxford Optronix, U.K.) was used as the oxygen sensor. A distance between material and light was set to 15 cm, and an amount of oxygen generated was measured in 0, 30, 60 and 120 min units using the oxygen sensor (reading time: 3 min).

As a result, it was confirmed that an amount of oxygen generated in the group having chloroplast inserted is higher than that of the control group (about 8% increase), and that a level of oxygen generated in the hypoxic state is more effective than that of the normoxic state. It was confirmed that an amount of oxygen generated in the group of chloroplast at a high concentration is decreased both in normoxic and hypoxic conditions (see FIG. 16).

13.2. Measurement of Amount of Oxygen Generated from Each Group According to Normoxic and Hypoxic States

An amount of oxygen generated from each group (free chloroplast, alginate-chloroplast hydrogel, and alginate-CTP-chloroplast hydrogel) according to exposure to light in normoxic and hypoxic states was confirmed by the following method.
Experimental group
(1) Free chloroplast
(2) Alginate-chloroplast hydrogel
(3) Alginate-CTP-chloroplast hydrogel
Concentration for each group (n=5, diluted with CIB, and a synthesis ratio was fixed at a molar ratio of alginate:peptide=1:5).
(1) Control (CIB)
(2) Chloroplast 1*102/ml
(3) Chloroplast 1*104/ml
(4) Chloroplast 1*106/ml
(5) Chloroplast 1*108/ml

Free chloroplast, alginate-chloroplast hydrogel, and alginate-CTP-chloroplast hydrogel were prepared by adding chloroplast to 96-well plate in accordance with the concentration. The 96-well plate prepared for each experimental group was implemented in normoxic and hypoxic states as follows.
Normoxic state: To implement a typical 21% oxygen environment
Hypoxic state: To implement a 1% oxygen environment by injecting a mixed gas (94% N2+5% CO2+1% O2) into a hypoxia chamber experiment instrument

An amount of oxygen generated from chloroplast according to light exposure in the normoxic and hypoxic states was measured using an oxygen sensor. At this time, OxyLite (Oxford Optronix, U.K.) was used as the oxygen sensor.

A distance between material and light was set to 5 cm, and an amount of oxygen generated was measured for six days using the oxygen sensor (at an interval of 24 hours). On the 5th day of the measurement, an amount of oxygen generated was measured by changing into a normoxic environment.

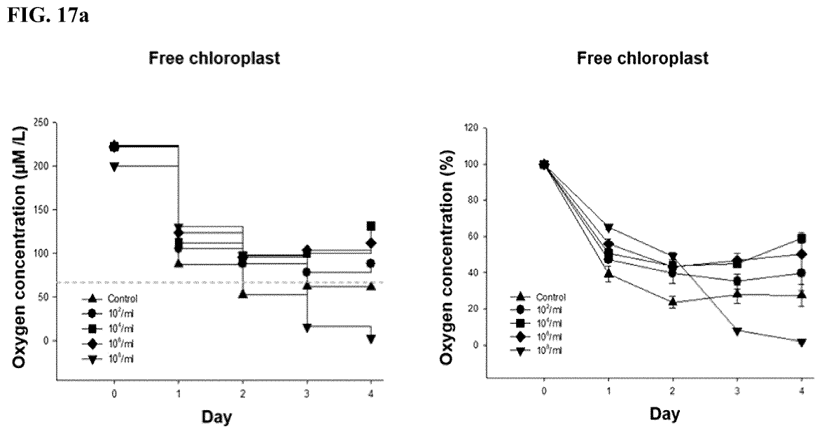

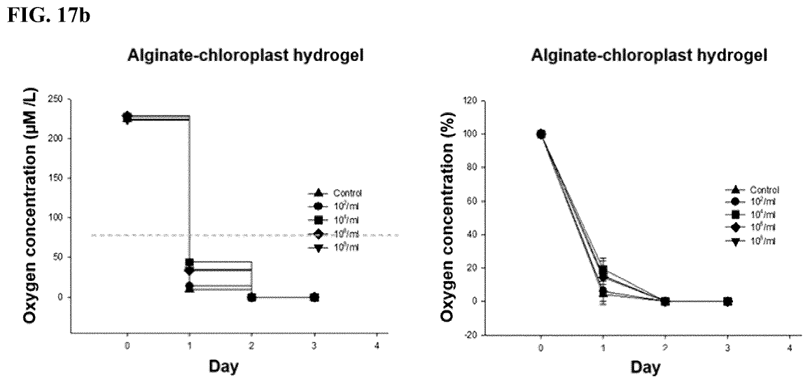

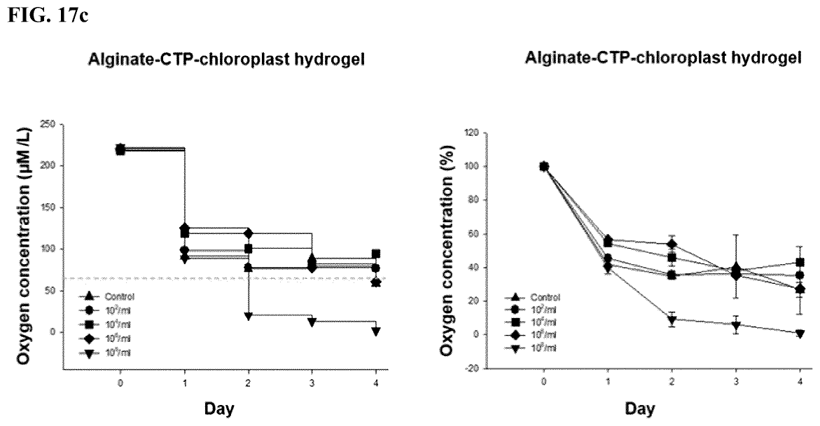

As a result, it was found that the group of free chloroplast maintains an oxygen concentration higher than a severe hypoxia level of 40 mmHg (69.5 μM/L) for five days. In addition, as a result of exposure to a normal oxygen concentration for 24 hours on the 5th day of the measurement, it was confirmed that most of the free chloroplast group is recovered to an initial oxygen concentration. However, as in the previous experimental results, it was found that a low level of oxygen concentration is measured in the control and the group of high concentration (108/ml). In particular, it was confirmed that the group of high concentration is not recovered to an oxygen concentration in the normoxic state on the 5th day of the measurement (see FIG. 17a). In the AC group, an oxygen concentration of all concentration groups was measured as 0 μM/L within 48 hours. Thus, it was confirmed that the AC is not capable of generating oxygen by chloroplast in a hypoxic state (see FIG. 17b). In addition, the ACC group showed an oxygen generation pattern similar to that of free chloroplast. Except for the control group and the group of high concentration (108/ml), it was found that the ACC group maintains an oxygen concentration of 40 mmHg (69.5 μM/L) or more, which is a severe hypoxia value. Overall, under hypoxic conditions, it was found that the ACC group shows an oxygen generation pattern similar to that of free chloroplast, and in particular, it was confirmed that oxygen is better generated at concentrations of 102/ml, 104/ml and 106/ml (see FIG. 17c).

### Example 14. Measurement of Hydrogen Peroxide Concentration

A reason why an amount of oxygen generated is decreased in the group of high-concentration chloroplast was confirmed by the following method.

The materials included (1) ABTS (548.68 g/mol), (2) HRP (peroxidase, 250 units/ml), (3) H202 solution (for standard curve, 8.943 M(9M/L)), and (4) chloroplast (0-3×108/ml).
Group
(1) Free chloroplast
(2) Alginate-CTP-chloroplast hydrogel
Concentration for each group (n=5)
※ Considering that concentration is diluted three folds when the sample is added, the concentration at the time of dilution was considered.
(1) Control (CIB)
(2) Chloroplast 3*102/ml
(3) Chloroplast 3*104/ml
(4) Chloroplast 3*106/ml
(5) Chloroplast 3*108/ml

Free chloroplast and alginate-CTP-chloroplast hydrogel were prepared by adding chloroplast to 96-well plate in accordance with the concentration.

After that, a sample required for ABTS assay was prepared as follows.
Preparation for ABTS 1.2 mM: 65.8 mg/100 ml->0.4 mM (diluted with tertiary distilled water)
Preparation for HRP 3 unit/ml: 1.2 mg/100 ml->1 unit/ml (diluted with tertiary distilled water)
Preparation for H2O2 solution for each concentration: At 3 mM, serial dilution was used to make 1, 0.5, 0.25 and 0.125 mM with tertiary distilled water.

To prepare a standard curve, 1 ml of HRP and 1 ml of H2O2 at each concentration were added, and then 1 ml of ABTS was added. HRP:H2O2:ABTS=1:1:1 was reacted, and ABTS was finally added. 200 μl of the solution was taken and transferred to 96-well plate, reacted for five minutes, and put into a plate reader to confirm absorbance at 405 nm. In order to confirm the H2O2 generated from chloroplast, 1 ml of chloroplast and 1 ml of HRP were added to a brown epen tube at each concentration, and then 1 ml of ABTS was added thereto. In this case, the brown tube was used because chloroplast is sensitive to light. After reacting for three minutes, the reaction mixture was subjected to centrifugation at 13500 RPM for two minutes. After that, 200 μl of the supernatant was taken, transferred to 96-well plate, and put into a plate reader to confirm absorbance at 405 nm. Due to a potential effect of absorbance caused by a color of chloroplast per se, only the supernatant was obtained.

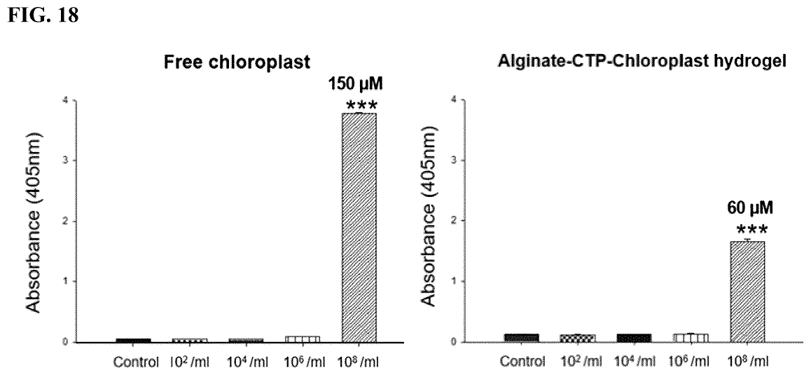

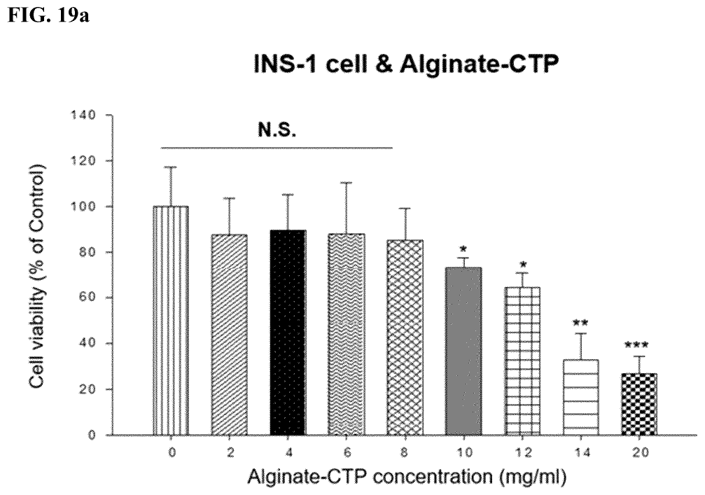

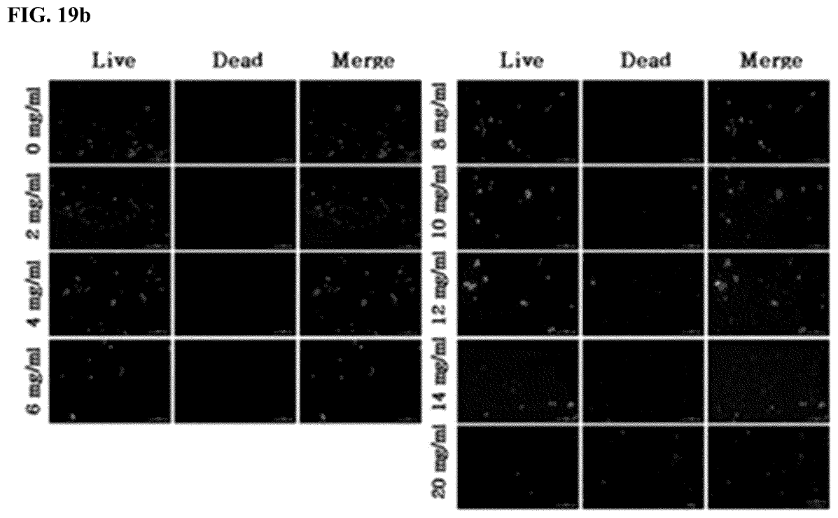

As a result of confirming an amount of hydrogen peroxide generated by the above method, both free chloroplast and ACC groups showed a significantly high level of hydrogen peroxide generated at a high concentration of chloroplast. Thus, it was confirmed that when a high concentration of chloroplast is treated, hydrogen peroxide is excessively generated to rather reduce an amount of oxygen generated (see FIG. 18). (N.S.: statistically no significant, *P≤0.05, **P≤0.01, ***P≤0.001, n=5, Results are shown as mean±S.E.M)

### Example 15. Evaluation of Toxicity of Alginate-CTP to INS-1 Cell

15.1. Evaluation of Toxicity of Alginate-CTP to INS-1 Cell Through CCK-8 Assay

It was confirmed whether the alginate-CTP solution is toxic to INS-1 cells by the following method.
Group (n=5, dissolved with media (RPMI+10% FBS), and a synthesis ratio was fixed at a molar ratio of alginate:peptide=1:5).
(1) Control (INS-1 cell+RPMI media)
(2) Alginate-CTP 2 mg/ml
(3) Alginate-CTP 4 mg/ml
(4) Alginate-CTP 6 mg/ml
(5) Alginate-CTP 8 mg/ml
(6) Alginate-CTP 10 mg/ml
(7) Alginate-CTP 12 mg/ml
(8) Alginate-CTP 14 mg/ml
(9) Alginate-CTP 20 mg/ml

INS-1 cells (3*10⁴/well) stabilized at 96-well plate for 24 hours were prepared (n=5). Each of the groups was treated with 100 μl of the alginate-CTP solution prepared at each concentration. After incubation for 24 hours, the alginate-CTP solution was suctioned. The resulting product was washed twice with PBS, treated with 100 μl of media (RPMI)+10 μl of CCK solution, and incubated by wrapping the foil for two hours, after which absorbance was measured at 450 nm using a reader.

As a result, 8 mg/ml, the concentration used in this study, did not show a significant difference from the viability of the control group (see FIG. 19a).

15.2. Evaluation of Toxicity of Alginate-CTP to INS-1 Cell Through Live & Dead Assay

It was confirmed whether the alginate-CTP solution is toxic to INS-1 cell line by the following method.
Group (n=5, dissolved with media (RPMI+10% FBS), and a synthesis ratio was fixed at a molar ratio of alginate:peptide=1:5).
(1) Control (INS-1 cell+RPMI media)
(2) Alginate-CTP 2 mg/ml
(3) Alginate-CTP 4 mg/ml
(4) Alginate-CTP 6 mg/ml
(5) Alginate-CTP 8 mg/ml
(6) Alginate-CTP 10 mg/ml
(7) Alginate-CTP 12 mg/ml
(8) Alginate-CTP 14 mg/ml
(9) Alginate-CTP 20 mg/ml

INS-1 cells (3*10⁴/well) stabilized at 96-well plate for 24 hours were prepared (n=5). Each of the groups was treated with 100 μl of the alginate-CTP solution prepared at each concentration and incubated for 24 hours, after which the alginate-CTP solution was suctioned. After that, the resulting product was washed twice with PBS, after which each well was treated with 100 μl of a reagent prepared with 1 μM calcein-AM and 1 μM ethidium homodimer-1 (EthD-1), and incubated for 10 minutes. Then, live cells (green fluorescence) and dead cells (red fluorescence) were confirmed using a fluorescence microscope, and a photograph was taken.

As a result, a similar level of green signal was confirmed. Thus, it was confirmed that the present concentration is not toxic to single cells (see FIG. 19b).

### Example 16. Preparation of Alginate-CTP-Chloroplast Hydrogel with Pancreatic Cell Encapsulated Therein

16.1. Isolation of Pancreatic Cell from Rat

Pancreatic cells secreting insulin were isolated from the pancreas of a rat by the following method.

The materials included (1) rat of strain SD (6-7 weeks old, male), (2) surgical tool (micro-scissors, forceps, etc.), (3) 1 mg/ml of collagenase (diluted with HBSS buffer at pH 7.8), and (4) 10 ml syringe.

The rat was euthanized by injecting 100% CO2 gas for 5-10 minutes, and it was confirmed that breathing or heartbreak was stopped. The outer skin and inner skin of the abdomen were removed, the liver was lifted to find the pancreas, and the pancreas was spread out with tongs to find duct (blood vessel), which is a part connected to the liver. After pulling the duct taut, collagenase, from which the extracellular matrix (ECM) may be removed by slightly scratching with scissors without breaking, was injected into a syringe at 10 ml per mouse through the duct. Swollen pancreas was isolated from the intestine, liver, stomach, and spleen using surgical tools. High-purity pancreatic cells were obtained from the pancreas through physical and chemical purification. Besides the pancreatic cells, various debris or impurities were removed through an optical microscope. For recovery and stabilization of the pancreatic cells, incubation was performed for 24 hours.

16.2. Preparation of Alginate-CTP-Chloroplast Hydrogel with Pancreatic Cell Encapsulated Therein

16.2.1. Preparation of Alginate-CTP-Chloroplast Hydrogel Encapsulating Pancreatic Cell with Alginate-CTP Composite Treated at Each Concentration

The isolated pancreatic cells were encapsulated in alginate-CTP-chloroplast hydrogel as follows.
Group (n=5, dissolved with media (RPMI+10% FBS), and a synthesis ratio was fixed at a molar ratio of alginate:peptide=1:5).
(1) Control (pancreatic cell+RPMI media)
(2) Alginate-CTP 8 mg/ml
(3) Alginate-CTP 16 mg/ml

The isolated pancreatic cells, which were stabilized for 24 hours, were washed twice with media (RPMI). Chloroplast solution isolated from spinach on the same day was prepared at a concentration of 4*106/ml. For a cell experiment, media (RPMI+10% FBS) was used when diluting chloroplast. 20 pancreatic cells (20IEQ) were well mixed with 250 μl of the prepared solution.

An alginate-CTP composite prepared at a concentration of 0, 8 and 16 mg/ml was prepared, mixed with the solution, and added into 100 mM CaCl2) solution to react for 30 minutes. Each of the prepared alginate-CTP-chloroplast hydrogel was placed in each well of 96-well plate (RPMI+10% FBS+1% PS, 200 μl) and incubated for 24 hours.

16.2.2. Preparation of Alginate-CTP-Chloroplast Hydrogel Encapsulating Pancreatic Cell with Chloroplast Treated at Each Concentration

The pancreatic cells were encapsulated in alginate-CTP-chloroplast hydrogel as follows.
Group (n=5, diluted with media (RPMI+10% FBS), and a synthesis ratio was fixed at a molar ratio of alginate:peptide=1:5).
(1) Control (pancreatic cell+RPMI media)
(2) Chloroplast 4*104/ml
(3) Chloroplast 4*106/ml

The pancreatic cells, which were stabilized for 24 hours, were washed twice with media (RPMI). Chloroplast isolated from spinach on the same day was prepared at a concentration of 4*106/ml. For a cell experiment, media (RPMI+10% FBS) was used when diluting chloroplast. 20 pancreatic cells (20IEQ) were well mixed with 250 μl of the isolated chloroplast solution. An alginate-CTP composite prepared at a concentration of 8 mg/ml was prepared. For a cell experiment, media (RPMI+10% FBS) was used when dissolving alginate-CTP. The above solution and the alginate-CTP composite were mixed, added in a 100 mM CaCl2) solution, and reacted for 30 minutes. Each of the prepared alginate-CTP-chloroplast hydrogel was placed in each well of 96-well plate (RPMI+10% FBS+1% PS, 200 μl) and incubated for 24 hours.

16.2.3. Preparation of Alginate-CTP-Chloroplast Hydrogel Encapsulating Pancreatic Cells with Chloroplast Treated at Each Concentration in Hypoxic Environment

The pancreatic cells were encapsulated in alginate-CTP-chloroplast hydrogel in a hypoxic environment as follows.
Group (n=6)
Diluted with media (RPMI+10% FBS).
A synthesis ratio was fixed at a molar ratio of alginate:peptide=1:5.
(1) Control 1 (RPMI media): Normoxic environment
(2) Control 2 (RPMI media): Hypoxic environment
(3) Pancreatic cell: Normoxic environment
(4) Pancreatic cell: Hypoxic environment
(5) Alginate-CTP hydrogel: Hypoxic environment
(6) Alginate-CTP-chloroplast (1*104/ml) hydrogel: Hypoxic environment
(7) Alginate-CTP-chloroplast (1*106/ml) hydrogel: Hypoxic environment

The pancreatic cells, which were isolated from an experiment animal (rat), were washed twice with media (RPMI). Chloroplast isolated from spinach on the same day was prepared at each concentration. For a cell experiment, media (RPMI+10% FBS) was used when diluting chloroplast. 20 pancreatic cells (20IEQ) were well mixed with 250 μl of the solution of the process 2. An alginate-CTP composite prepared at a concentration of 8 mg/ml was prepared. For a cell experiment, media (RPMI+10% FBS) was used when dissolving alginate-CTP. The above solutions were mixed and added to a 100 mM CaCl2) solution, and reacted for 30 minutes. Each of the prepared alginate-CTP-chloroplast hydrogel at each concentration was placed in each well of 6-well plate (RPMI+10% FBS+1% PS, 1 ml). Above groups (1), (2), (3) and (4) were added to each well of 6-well plate (RPMI+10% FBS+1% PS, 200 μl), and the 6-well plate was implemented in normoxic and hypoxic states as follows and incubated for 24 hours.
Normoxic state: To implement a typical 21% oxygen environment
Hypoxic state: To implement a 1% oxygen, 37° C. environment by injecting a mixed gas (94% N2+5% CO2+1% O2) into a temperature-controlled desiccator experiment instrument

### Example 17. Evaluation of Toxicity of Pancreatic Cell Encapsulated in Alginate-CTP-Chloroplast Hydrogel

17.1. Evaluation of Toxicity of Pancreatic Cell Encapsulated in Alginate-CTP-Chloroplast Hydrogel with Alginate-CTP Composite Treated at Each Concentration

17.1.1. Evaluation of Toxicity Through CCK-8 Assay

Viability of pancreatic cells encapsulated in alginate-CTP-chloroplast hydrogel with alginate-CTP composite treated at each concentration was confirmed by the following method.
Group (n=5, dissolved with media (RPMI+10% FBS), and a synthesis ratio was fixed at a molar ratio of alginate:peptide=1:5).
(1) Control (pancreatic cell+RPMI media)
(2) Alginate-CTP 8 mg/ml
(3) Alginate-CTP 16 mg/ml

Alginate-CTP-chloroplast hydrogel, in which pancreatic cells incubated in 96-well plate for 24 hours were encapsulated and alginate-CTP was treated at each concentration, was prepared. All hydrogel was washed twice with PBS and treated with 100 μl of media (RPMI)+10 μl of CCK solution. After incubation by wrapping the foil for two hours, absorbance was measured at 450 nm using a reader.

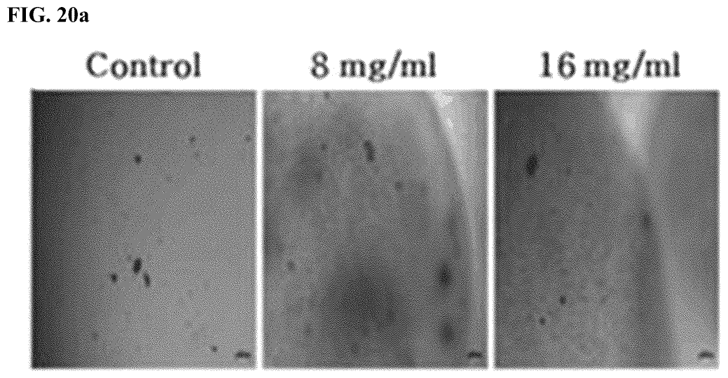

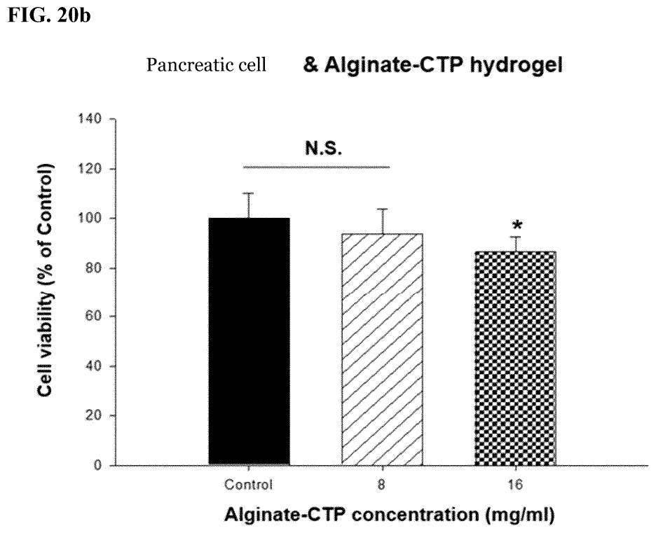

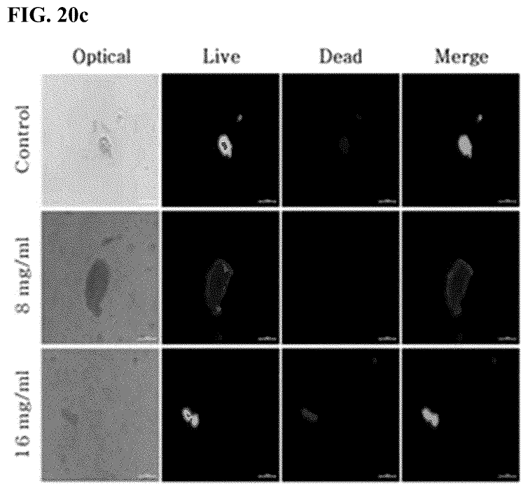

As a result, the concentration used in this study, 8 mg/ml, did not show a significant difference from the viability of the control group (see FIGS. 20a and 20b).

17.1.2. Evaluation of Toxicity Through Live & Dead Assay

Viability of pancreatic cells encapsulated in alginate-CTP-chloroplast hydrogel with alginate-CTP composite treated at each concentration was confirmed by the following method.
Group (n=5, dissolved with media (RPMI+10% FBS), and a synthesis ratio was fixed at a molar ratio of alginate:peptide=1:5).
(1) Control (pancreatic cell+RPMI media)
(2) Alginate-CTP 8 mg/ml
(3) Alginate-CTP 16 mg/ml

Alginate-CTP-chloroplast hydrogel, in which pancreatic cells incubated in 96-well plate for 24 hours were encapsulated and alginate-CTP was treated at each concentration, was prepared. All hydrogel was washed twice with PBS, and then each well was treated with 100 μl of a reagent prepared with 1 μM calcein-AM and 1 μM ethidium homodimer-1 (EthD-1), and incubated for 30 minutes. Live cells (green fluorescence) and dead cells (red fluorescence) were confirmed using a fluorescence microscope, and then a photograph was taken.

As a result, a similar level of green signal was confirmed even through live & dead assay (see FIG. 20c). Thus, it was confirmed that the present concentration is not toxic to single cells as well as cell groups.

17.2. Evaluation of Toxicity of Pancreatic Cell Encapsulated in Alginate-CTP-Chloroplast Hydrogel with Chloroplast Treated at Each Concentration

17.2.1. Evaluation of Toxicity Through CCK-8 Assay

Viability of pancreatic cells encapsulated in alginate-CTP-chloroplast hydrogel with chloroplast treated at each concentration was confirmed as follows.
Group (n=5, diluted with media (RPMI+10% FBS), and a synthesis ratio was fixed at a molar ratio of alginate:peptide=1:5).
(1) Control (pancreatic cell+RPMI media)
(2) Chloroplast 1*104/ml
(3) Chloroplast 1*106/ml

Alginate-CTP-chloroplast hydrogel, in which pancreatic cells incubated in 96-well plate for 24 hours were encapsulated and chloroplast was treated at each concentration, was prepared. All hydrogel was washed twice with PBS and treated with 100 μl of media (RPMI)+10 μl of CCK solution. After incubation by wrapping the foil for two hours, absorbance was measured at 450 nm using a reader.

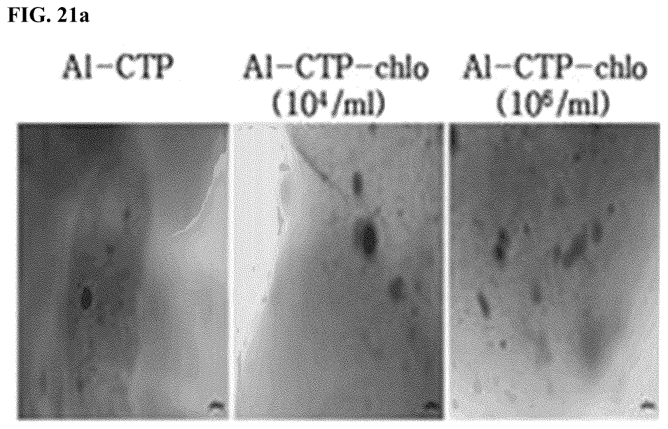

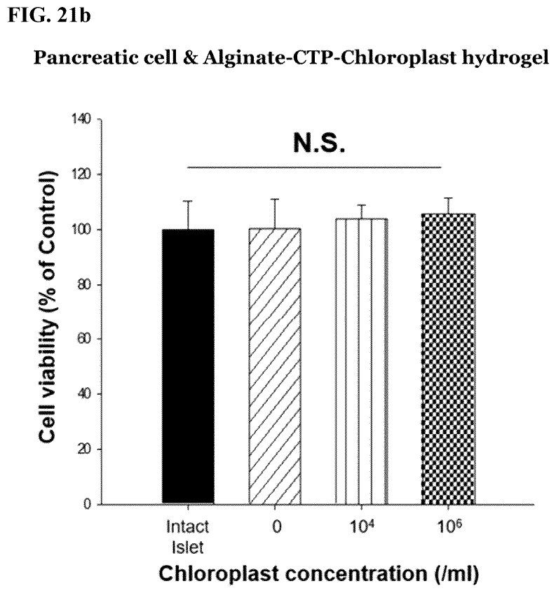

As a result, the viability of all concentration groups was not significantly different from that of the control group regardless of the concentration of chloroplast (see FIGS. 21a and 21b).

17.2.2. Evaluation of Toxicity Through Live & Dead Assay

Viability of pancreatic cells encapsulated in alginate-CTP-chloroplast hydrogel with chloroplast treated at each concentration was confirmed as follows.
Group (n=5, diluted with media (RPMI+10% FBS), and a synthesis ratio was fixed at a molar ratio of alginate:peptide=1:5).
(1) Control (pancreatic cell+RPMI media)
(2) Chloroplast 1*104/ml
(3) Chloroplast 1*106/ml

Alginate-CTP-chloroplast hydrogel, in which pancreatic cells incubated in 96-well plate for 24 hours were encapsulated and chloroplast was treated at each concentration, was prepared. All hydrogel was washed twice with PBS, and then each well was treated with 100 μl of a reagent prepared with 1 μM calcein-AM and 1 μM ethidium homodimer-1 (EthD-1). Incubation was performed for 30 minutes, live cells (green fluorescence) and dead cells (red fluorescence) were confirmed using a fluorescence microscope, and then a photograph was taken.

As a result, a strong green signal was confirmed through live & dead assay (see FIG. 21c). Thus, it was confirmed that alginate-CTP-chloroplast hydrogel is a material non-toxic to pancreatic cells regardless of the concentration of chloroplast.

17.3. Evaluation of Toxicity of Pancreatic Cell Encapsulated in Alginate-CTP-Chloroplast Hydrogel with Chloroplast Treated at Each Concentration in Hypoxic Environment (CCK-8 Assay)

It was performed to confirm the viability of pancreatic cells encapsulated in alginate-CTP-chloroplast hydrogel with chloroplast treated at each concentration in a hypoxic environment as follows.
Group (n=6)
Diluted with media (RPMI+10% FBS).
A synthesis ratio was fixed at a molar ratio of alginate:peptide=1:5.
(1) Control 1 (RPMI media): Normoxic environment
(2) Control 2 (RPMI media): Hypoxic environment
(3) Pancreatic cell: Normoxic environment
(4) Pancreatic cell: Hypoxic environment
(5) Alginate-CTP hydrogel: Hypoxic environment
(6) Alginate-CTP-chloroplast (1*104/ml) hydrogel: Hypoxic environment
(7) Alginate-CTP-chloroplast (1*106/ml) hydrogel: Hypoxic environment

Alginate-CTP-chloroplast hydrogel, in which pancreatic cells incubated in normoxic and hypoxic environments for 24 hours were encapsulated and chloroplast was treated at each concentration, was prepared for each group. All groups were washed twice with PBS, treated with 100 μl of media (RPMI)+10 μl of CCK solution, and incubated by wrapping the foil for two hours, after which absorbance was measured at 450 nm using a reader. After that, an absorbance value was corrected through an amount of DNA for each experimental group using Quant-iT™ PicoGreen™ dsDNA Assay Kits.

As a result, due to exposure of pancreatic cells to a hypoxic environment for a long time, it can be confirmed that viability of pancreatic cells may not be significantly decreased compared to the IN group except for the IH group. It could be confirmed that viability of pancreatic cells is decreased about 18% in the IH group due to exposure to the hypoxic environment for 24 hours, representing that the hypoxic environment is not appropriate for culturing the pancreatic cells. It was confirmed that alginate-CTP-chloroplast (groups 4, 6) has a lower cell viability than that of the IN group, representing an insignificant result. With regard to alginate-CTP-chloroplast hydrogel, it means that the alginate-CTP-chloroplast material is not toxic to pancreatic cells regardless of a concentration of chloroplast. In addition, it was found that groups 0, 4 and 6 show higher cell viability than that of the IH group in a hypoxic environment, representing that pancreatic cells encapsulated in alginate-CTP-chloroplast hydrogel may have an appropriate condition for culturing in a hypoxic environment (see FIG. 32).

### Example 18. Measurement of Amount of Oxygen Generated from Alginate-CTP-Chloroplast Hydrogel with Pancreatic Cells Encapsulated Therein and with Chloroplast Treated at Each Concentration

An amount of oxygen generated from alginate-CTP-chloroplast hydrogel with pancreatic cells encapsulated therein and with chloroplast treated at each concentration was confirmed.
Group (n=5, diluted with media (RPMI+10% FBS), and a synthesis ratio was fixed at a molar ratio of alginate:peptide=1:5).
(1) Control (pancreatic cell+RPMI media)
(2) Chloroplast 1*104/ml
(3) Chloroplast 1*106/ml

Alginate-CTP-chloroplast hydrogel with pancreatic cells encapsulated therein and with chloroplast treated at each concentration was prepared. One hydrogel of process 1 was added to each well of an oxygen plate reader well plate (24 well), and an amount of oxygen generated was measured using an oxygen sensor at 0, 6, 12, 18, 24 and 30 hours while incubation was performed for 30 hours. SDR SensorDish (PreSens, Germany) was used as the oxygen sensor, and SDR SensorDish-dedicated well plate was used as an oxygen plate reader well plate (24 well).

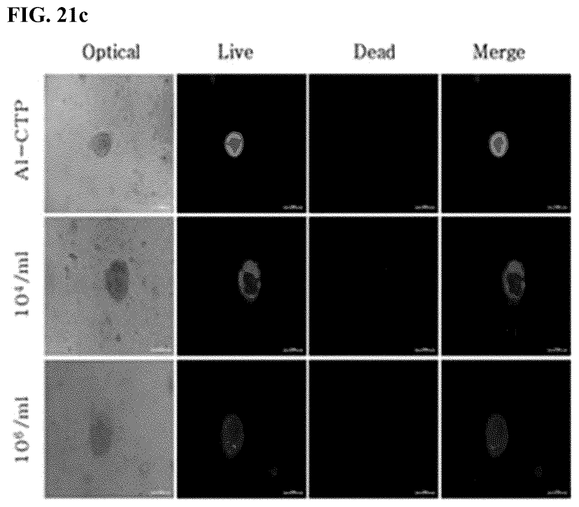

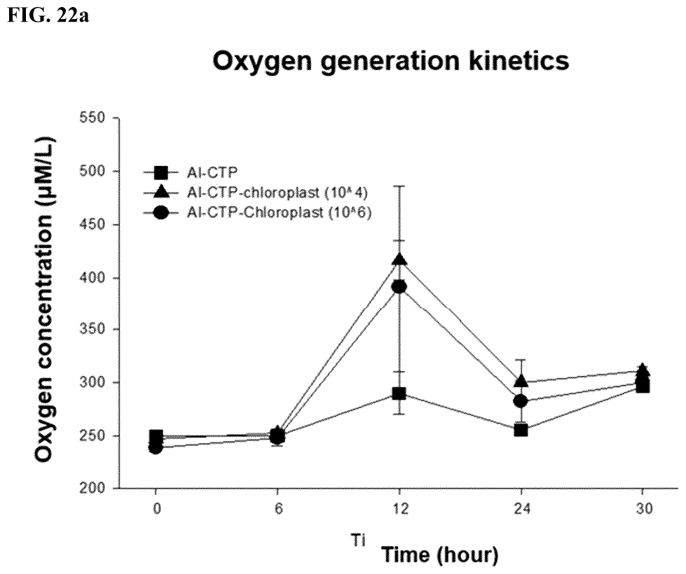

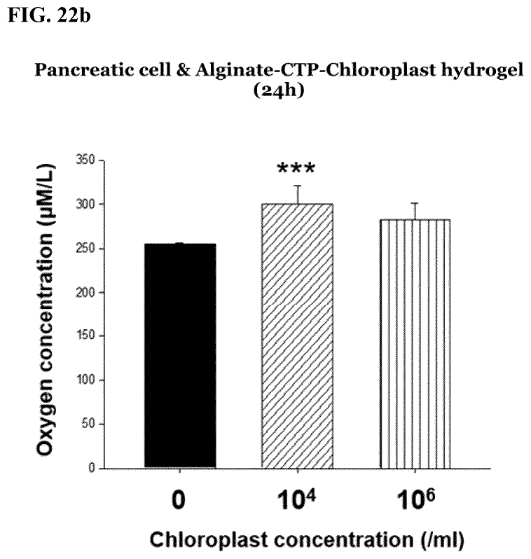

As a result, an amount of oxygen generated was steadily increased with the passage of time, and a higher level of oxygen generated was observed in a group having chloroplast inserted than in a control group without chloroplast (see FIG. 22a). In addition, as a result of measurement at 24 hours, it was confirmed that an amount of oxygen generated from alginate-CTP-chloroplast hydrogel, in which chloroplast is inserted at a concentration of 104/ml, is significantly higher than that of the control group without chloroplast (see FIG. 22b).

### Example 19. Confirmation of Functionality of Pancreatic Cell Encapsulated in Alginate-CTP-Chloroplast Hydrogel

19.1. Confirmation of Functionality of Pancreatic Cell Encapsulated in Alginate-CTP-Chloroplast Hydrogel with Chloroplast Treated at Each Concentration

19.1.1. Confirmation Through Glucose-Stimulated Insulin Secretion (GSIS) Assay

An insulin secretion function of pancreatic cells encapsulated in alginate-CTP-chloroplast hydrogel with chloroplast treated at each concentration was confirmed as follows.

The materials included (1) low glucose (2.8 mM, diluted with Krebs Ringer buffered HEPES (pH 7.4)) and (2) high glucose (20.2 mM, diluted with Krebs Ringer buffered HEPES (pH 7.4)).
Group (n=5, diluted with media (RPMI+10% FBS), and a synthesis ratio was fixed at a molar ratio of alginate:peptide=1:5).
(1) Control (pancreatic cell+RPMI media)
(2) Chloroplast 1*104/ml
(3) Chloroplast 1*106/ml

Alginate-CTP-chloroplast hydrogel, in which pancreatic cells incubated in 96-well plate for 24 hours were encapsulated and chloroplast was treated at each concentration, was prepared. All hydrogel was washed twice with PBS, and then low glucose (2.8 mM) was dispensed by 200 ul in each well and incubated for 30 minutes. Low glucose (2.8 mM) was again dispensed by 200 μl to each well and incubated for two hours, and then the present low glucose solution was recovered. In addition, high glucose (20.2 mM) was dispensed by 200 μl to each well and incubated for two hours, and then the high glucose solution present was recovered. An amount of insulin secretion was measured and calculated based on low glucose and high glucose solutions recovered using insulin enzyme-linked immunosorbent (ELISA) assay.

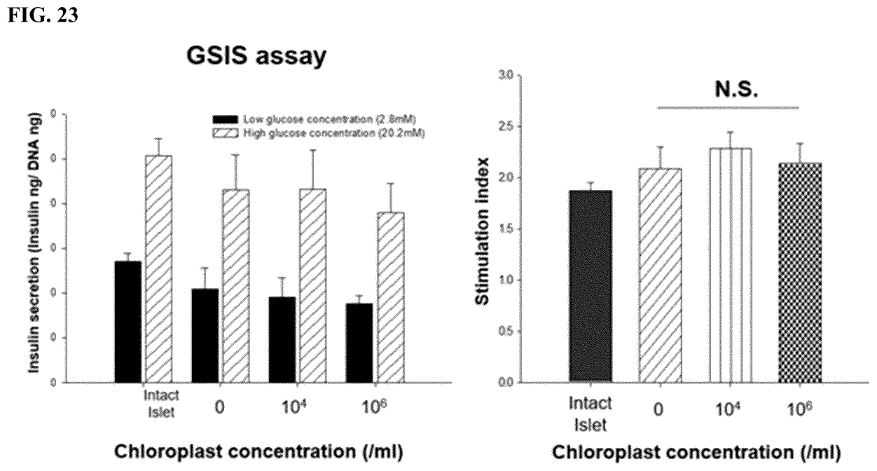

As a result, the pancreatic cells encapsulated in alginate-CTP-chloroplast hydrogel showed an amount of insulin secretion similar to that of the control group (intact islet) regardless of the concentration of chloroplast (see FIG. 23). The results indicate that alginate-CTP-chloroplast hydrogel well maintains the insulin secretion ability of the encapsulated pancreatic cells and does not affect the function of the cells.

19.1.2. Confirmation Through dsDNA Assay

An amount of insulin secreted from pancreatic cells encapsulated in alginate-CTP-chloroplast hydrogel with chloroplast treated at each concentration was confirmed as follows.
Group (n=5, diluted with media (RPMI+10% FBS), and a synthesis ratio was fixed at a molar ratio of alginate:peptide=1:5).
(1) Control (pancreatic cell+RPMI media)
(2) Chloroplast 1*104/ml
(3) Chloroplast 1*106/ml

Alginate-CTP-chloroplast hydrogel, in which pancreatic cells incubated in 96-well plate for 24 hours were encapsulated and chloroplast was treated at each concentration, was prepared. All hydrogel was washed twice with PBS, and 0.1 M EDTA (pH 7.8) solution was dispensed by 200 μl into each well and hydrogel was crushed through sufficient pipetting. At this time, it was performed to take out the pancreatic cells present in hydrogel. Each of the solution was recovered into 1.5 ml tube and subjected to centrifugation at 1800 RPM for two minutes. The supernatant was removed and then pellets (pancreatic cells) were treated with 1 ml of RIPA buffer each. An amount of insulin secreted by pancreatic cells present in each well (alginate-CTP-chloroplast hydrogel) was corrected through dsDNA assay.

19.2. Confirmation of Functionality of Pancreatic Cell Encapsulated in Alginate-CTP-Chloroplast Hydrogel with Chloroplast Treated at Each Concentration in Hypoxic Environment

19.2.1. Confirmation Through Glucose-Stimulated Insulin Secretion (GSIS) Assay

An insulin secretion function of pancreatic cells encapsulated in alginate-CTP-chloroplast hydrogel with chloroplast treated at each concentration in a hypoxic environment was confirmed as follows.
Materials
(1) Low glucose (2.8 mM, diluted with Krebs Ringer buffered HEPES (pH 7.4))
(2) High glucose (20.2 mM, diluted with Krebs Ringer buffered HEPES (pH 7.4))
Group (n=6)
Diluted with media (RPMI+10% FBS).
A synthesis ratio was fixed at a molar ratio of alginate:peptide=1:5.
(1) Control 1 (RPMI media): Normoxic environment
(2) Control 2 (RPMI media): Hypoxic environment
(3) Pancreatic cell: Normoxic environment
(4) Pancreatic cell: Hypoxic environment
(5) Alginate-CTP hydrogel: Hypoxic environment
(6) Alginate-CTP-chloroplast (1*104/ml) hydrogel: Hypoxic environment
(7) Alginate-CTP-chloroplast (1*106/ml) hydrogel: Hypoxic environment

Alginate-CTP-chloroplast hydrogel, in which pancreatic cells incubated in normoxic and hypoxic environments for 24 hours were encapsulated and chloroplast was treated at each concentration, was prepared for each group. All groups were washed twice with PBS, and then low glucose (2.8 mM) was dispensed by 200 ul in each well and incubated for 30 minutes. Low glucose (2.8 mM) was again dispensed by 200 μl to each well and incubated for two hours, and then the present low glucose solution was recovered. High glucose (20.2 mM) was dispensed by 200 μl to each well and incubated for two hours, and then the high glucose solution present was recovered. An amount of insulin was measured and calculated based on low glucose and high glucose solutions recovered using insulin enzyme-linked immunosorbent (ELISA) assay.

As a result, it can be confirmed that an insulin secretion ability of pancreatic cells may not be significantly decreased compared to the IN group except for the IH group. It is confirmed that the IH group has a reduced insulin secretion ability in pancreatic cells due to exposure to a hypoxic environment for 24 hours. Alginate-CTP-chloroplast group shows a glucose stimulation index (SI) of pancreatic cells similar to that of the IN group, representing that insulin secretion ability was not reduced even though alginate-CTP-chloroplast group (groups 4, 6) was exposed to a hypoxic environment. In addition, it was found that groups 0, 4, and 6 show a significantly higher insulin secretion ability than that of the IH group in a hypoxic environment, representing that pancreatic cells encapsulated in alginate-CTP-chloroplast hydrogel did not have a reduced insulin secretion ability even though the pancreatic cells were cultured in a hypoxic environment, and may have an appropriate condition for culturing in a hypoxic environment.

19.2.2. Confirmation Through dsDNA Assay

An amount of insulin secreted from pancreatic cells encapsulated in alginate-CTP-chloroplast hydrogel with chloroplast treated at each concentration in a hypoxic environment was corrected with a size of pancreatic cells as follows.
Group (n=6)
Diluted with media (RPMI+10% FBS).
A synthesis ratio was fixed at a molar ratio of alginate:peptide=1:5.
(1) Control 1 (RPMI media): Normoxic environment
(2) Control 2 (RPMI media): Hypoxic environment
(3) Pancreatic cell: Normoxic environment
(4) Pancreatic cell: Hypoxic environment
(5) Alginate-CTP hydrogel: Hypoxic environment
(6) Alginate-CTP-chloroplast (1*104/ml) hydrogel: Hypoxic environment
(7) Alginate-CTP-chloroplast (1*106/ml) hydrogel: Hypoxic environment

Alginate-CTP-chloroplast hydrogel, in which pancreatic cells incubated in normoxic and hypoxic environments for 24 hours were encapsulated and chloroplast was treated at each concentration, was prepared for each group. All groups were washed twice with PBS, and 0.1 M EDTA (pH 7.8) solution was dispensed by 200 μl into each well and cells and hydrogel were crushed through sufficient pipetting. (It was performed to take out the pancreatic cells present in hydrogel.) Each of the solution was recovered into 1.5 ml tube and subjected to centrifugation at 1800 RPM for two minutes. The supernatant was removed and then pellets (pancreatic cells) were treated with 1 ml of RIPA buffer each. An amount of insulin secreted by pancreatic cells present in each well (alginate-CTP-chloroplast) was corrected through dsDNA assay.

### Example 20. Measurement of Amount of Oxygen Generated from Chloroplast at Each Concentration Under Light (660 nm) Condition

20.1. Measurement of Amount of Oxygen Generated Using Hypoxia Chamber

An amount of oxygen generated from chloroplast at each concentration in a hypoxic environment depending on the presence or absence of light (660 nm) was confirmed as follows.
Group
(1) Light (660 nm) exposure group
(2) Light (660 nm) non-exposure group
Concentration for each group (n=6)
1) to 4) were diluted with CIB.
1) Control (CIB)
2) Chloroplast 1*106/ml
3) Chloroplast 1*107/ml
4) Chloroplast 1*108/ml
5) to 6) were diluted with media (RPMI+10% FBS).
5) Control (Media)
6) Chloroplast 1*106/ml
7) Chloroplast 1*107/ml
8) Chloroplast 1*108/ml

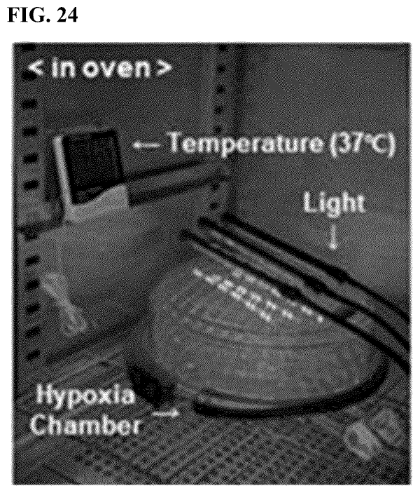

Chloroplast at each concentration was dispensed by 1 ml each to an oxygen plate reader (24 wells) in accordance with each group, and the plate was implemented in a hypoxic state as follows.
Hypoxic state: To implement a 1% oxygen environment by injecting a mixed gas (94% N2+5% CO2+1% O2) into a hypoxia chamber experiment instrument (see FIG. 24)

A distance between material and light was set to 5 cm, which was applied only to a light exposure group and a light non-exposure group was performed without light. After that, an amount of oxygen generated from chloroplast depending on the presence or absence of light was measured using an oxygen sensor (reading time: 30 min). At this time, SDR SensorDish (PreSens, Germany) was used as the oxygen sensor.

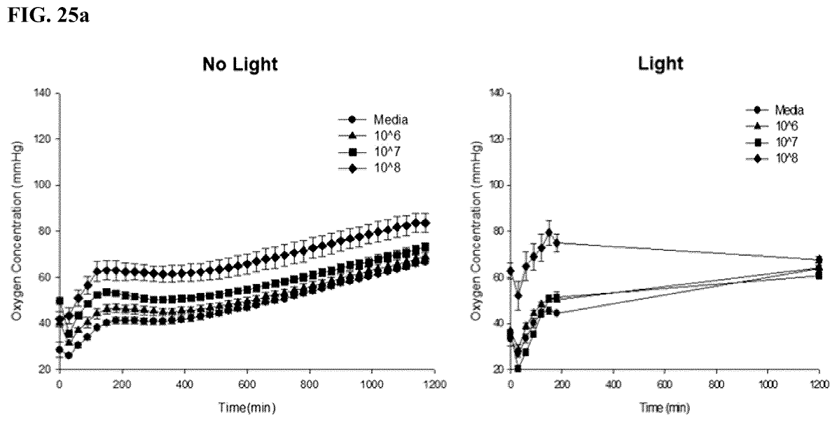

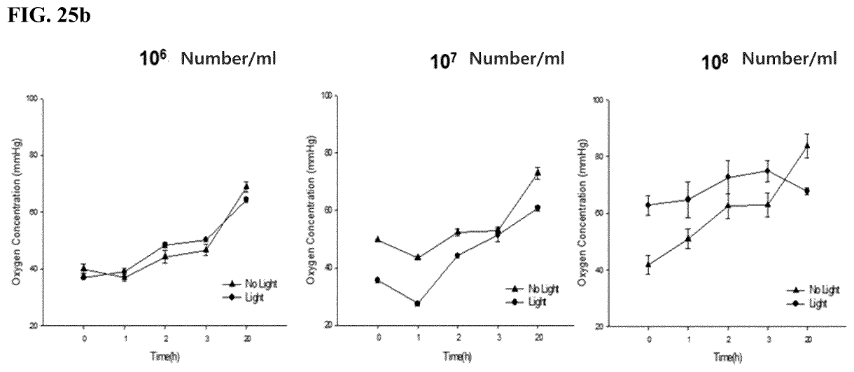

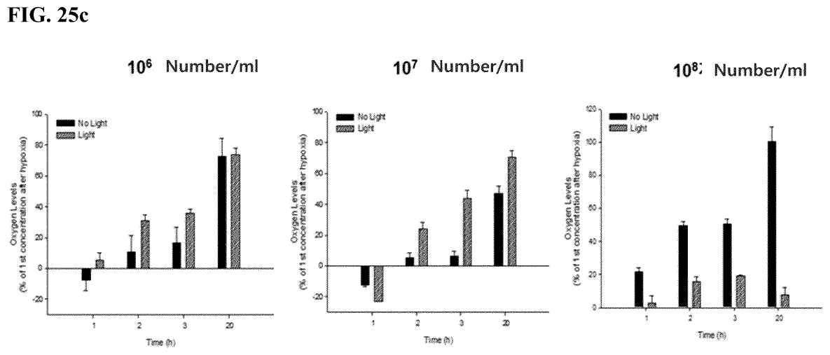

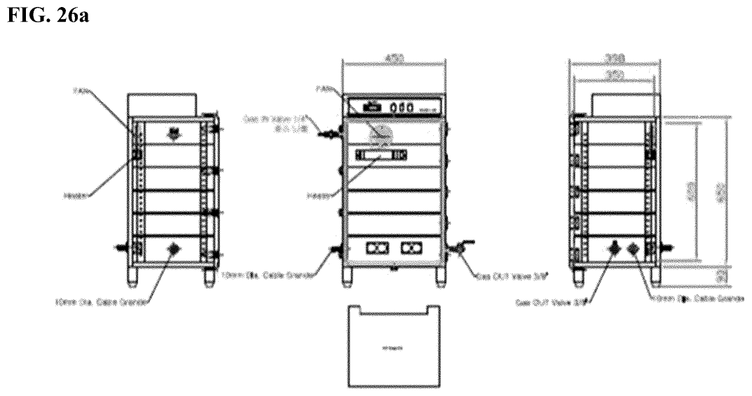

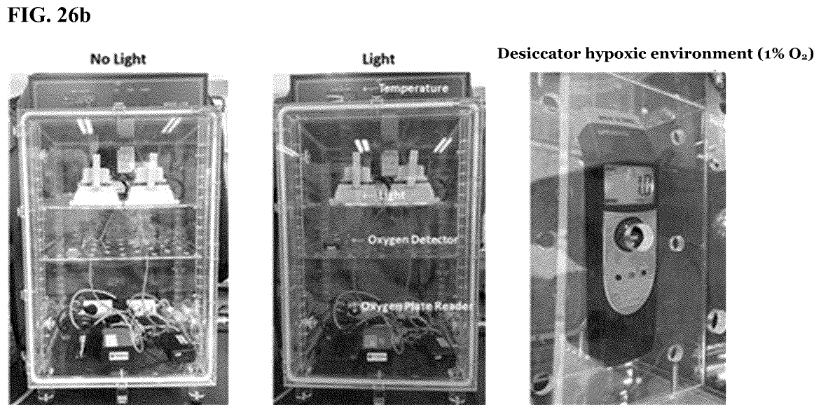

As a result, it can be confirmed that an initial oxygen concentration of chloroplast with a hypoxic environment implemented is different according to a concentration of chloroplast for each group, representing that an amount of oxygen generated at the concentration of chloroplast is different (see FIG. 26a). In addition, with regard to the effect of light (660 nm), it could be confirmed that a concentration of oxygen in 108/ml chloroplast is significantly increased in the group exposed to light for 20 hours compared to the group not exposed to light (see FIGS. 25b and 25c).

20.2. Measurement of amount of oxygen generated using temperature-controlled desiccator

An amount of oxygen generated from chloroplast in a hypoxic environment depending on the presence or absence of light (660 nm) was confirmed as follows.
Group
(1) Light (660 nm) exposure group (Light)
(2) Light (660 nm) non-exposure group (No Light)
Concentration at each concentration (n=24, diluted with media (RPMI+10% FBS)
(1) Control (Media)
(2) Chloroplast 1*108/ml

Chloroplast at each concentration was dispensed by 1 ml each to an oxygen plate reader (24 wells) in accordance with each group. The plate was implemented in a hypoxic state as follows.
Hypoxic state: To implement a 1% oxygen, 37° C. environment by injecting a mixed gas (94% N2+5% CO2+1% O2) into a temperature-controlled desiccator (see FIG. 26).

A distance between material and light was set to 5 cm, which was applied only to a light exposure group and a light non-exposure group was performed without light. An amount of oxygen generated from chloroplast depending on the presence or absence of light was measured using an oxygen sensor for 20 hours. (reading time: 30 min) At this time, SDR SensorDish (PreSens, Germany) was used as the oxygen sensor.

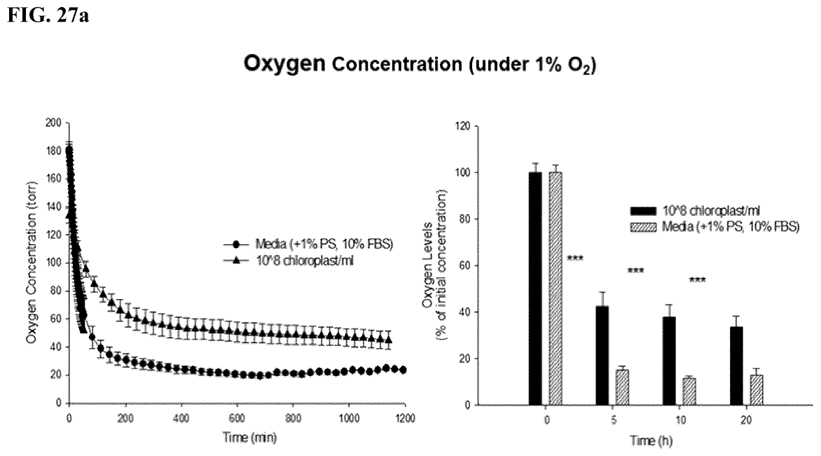

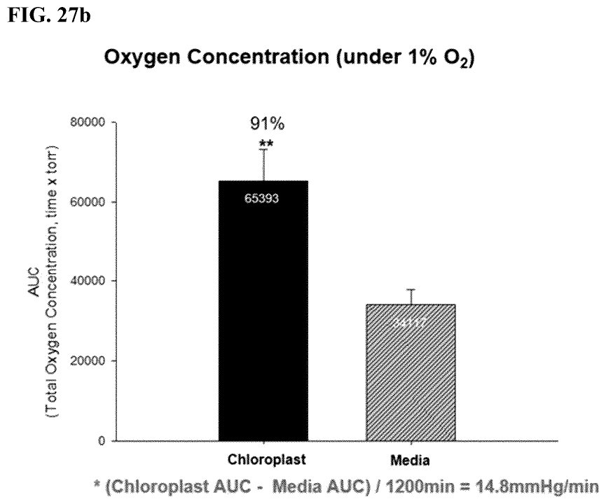

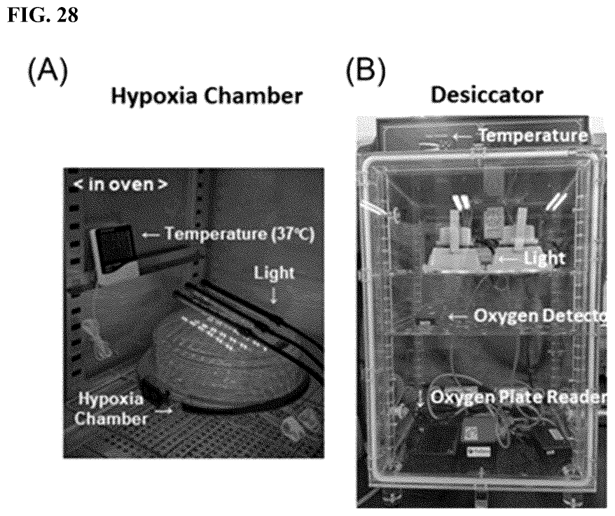

As a result, it could be confirmed that an oxygen concentration is significantly higher in chloroplast (108/ml) compared to the control media (RPMI+1% PS, 10% FBS) (see FIG. 27a). In addition, when comparing a total oxygen concentration for 20 hours (AUC), it could be confirmed that an oxygen concentration in the chloroplast group is about 48% higher than that of the control group (see FIG. 27b). If the oxygen concentration is converted into an amount of oxygen generated per minute (total amount of oxygen generated/1200 min), 108/ml chloroplast at 37° C. means that an amount of oxygen corresponding to 14.8 mmHg per minute on average may be generated more compared to the control group. The above result means that chloroplast has the effect of generating oxygen compared to the control group.

### Example 21. Measurement of Amount of Oxygen Generated from Chloroplast in Hypoxic Environment

21.1. Measurement of Amount of Oxygen Generated According to Temperature Condition

An amount of oxygen generated from chloroplast in a hypoxic environment according to a temperature was confirmed as follows.
Group (n=24)
Diluted with media (RPMI+10% FBS).
Concentration of chloroplast is fixed to 1*108/ml.
(1) 25° C.
(2) 37° C.

Chloroplast, of which a temperature was adjusted in advance, was prepared and dispensed by 1 ml each to an oxygen plate reader (24 wells) in accordance with each group. The plate was implemented in a hypoxic state as follows.
Hypoxic state: To implement a 1% oxygen environment by injecting a mixed gas (94% N2+5% CO2+1% O2) into a temperature-controlled desiccator

An amount of oxygen generated from chloroplast according to a temperature was measured using an oxygen sensor. At this time, SDR SensorDish (PreSens, Germany) was used as the oxygen sensor.

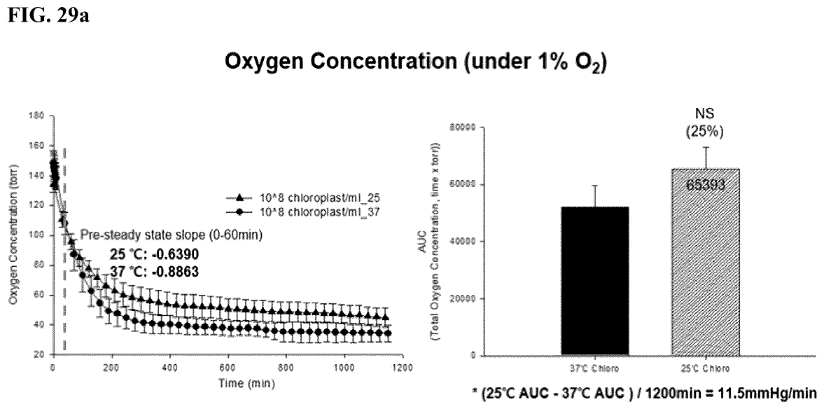

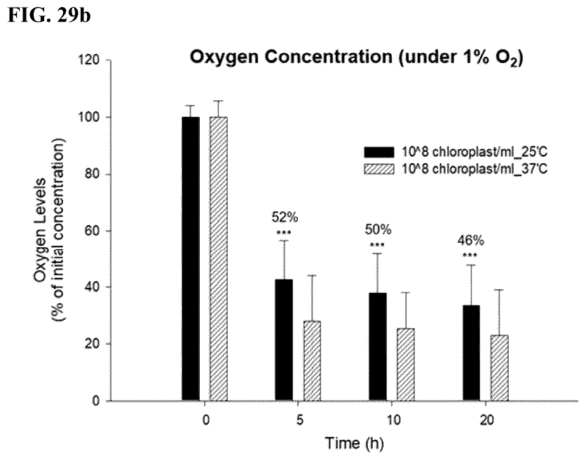

As a result, it was confirmed that 25° C. chloroplast shows a small amount of change in the oxygen concentration over time, representing that it takes a long time to reach the same concentration as that of 37° C. chloroplast (see FIG. 29a). In addition, when comparing the AUC to compare a total amount of oxygen generated for 20 hours, it was found that 25° C. chloroplast generates about 25% more oxygen compared to 37° C. chloroplast, which is an insignificant result.

Furthermore, when comparing a degree of decrease in the oxygen concentration for each time (0, 5, 10 and 20 hours) in the graph with an initial oxygen concentration in each group, it was confirmed that a degree of decrease in 25° C. chloroplast might be lower than that at 37° C. chloroplast (see FIG. 29b). It means that the 25° C. environment may maintain a photosynthetic activity of chloroplast longer than that of the 37° C. environment, representing that the 25° C. environment may lead to more oxygen generation.

From the above results, it was confirmed that chloroplast may generate oxygen to the maximum when a surrounding environment is given as 25° C. However, referring to [The structural integrity of the islet in vitro: the effect of incubation temperature; A Ilieva, S Yuan], it was confirmed that pancreatic cells cultured at 24° C. lose morphology thereof and about 40% of the cells are killed when cultured for 12 days. In addition, with regard to an insulin immune activity at 24° C., it was confirmed that the homogeneity of distribution is gradually decreased, and on the 12th day, an insulin immune active region is remarkably decreased to 19±9% of the total pancreatic cells.

As a result, considering that an insulin secretion ability of the pancreatic cells used in a future experiment would be reduced, a subsequent experiment was performed in the environment of 37° C.

21.2. Measurement of Amount of Oxygen Generated According to Light Condition

21.2.1. Measurement of Amount of Oxygen Generated According to Intensity (Lux) of Light (660 nm)

An amount of oxygen generated from chloroplast in a hypoxic environment according to intensity (lux) of light (660 nm) was confirmed as follows.
Group (n=24)
Diluted with media (RPMI+10% FBS).
Concentration of chloroplast is fixed to 1*108/ml.
A temperature is fixed to 37° C.
(1) Light 1500 lux
(2) Light 1000 lux
(3) Light 500 lux

Chloroplast at a concentration of 1*108/ml was dispensed by 1 ml each to an oxygen plate reader (24 wells). The plate was implemented in a hypoxic state as follows.
Hypoxic state: To implement a 1% oxygen, 37° C. environment by injecting a mixed gas (94% N2+5% CO2+1% O2) into a temperature-controlled desiccator.

A distance between material and light was set to 5 cm, 10 cm and 15 cm, and an amount of oxygen generated from chloroplast according to an intensity of light was measured using an oxygen sensor. The intensity of light reaching the material may be changed to 1500 lux, 1000 lux and 500 lux according to the distance between the material and the light, which is 5 cm, 10 cm and 15 cm. At this time, SDR SensorDish (PreSens, Germany) was used as the oxygen sensor.

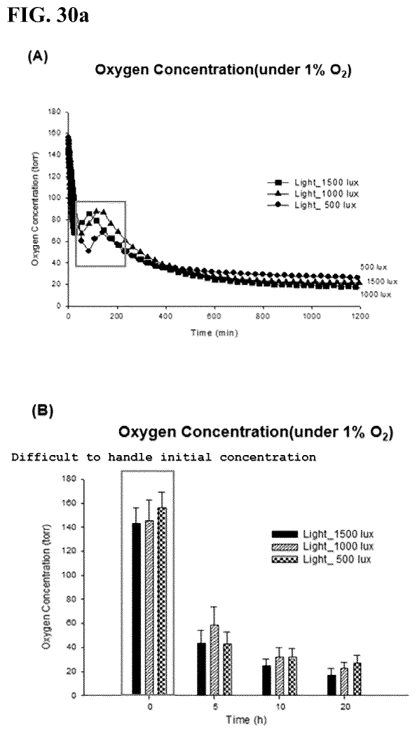

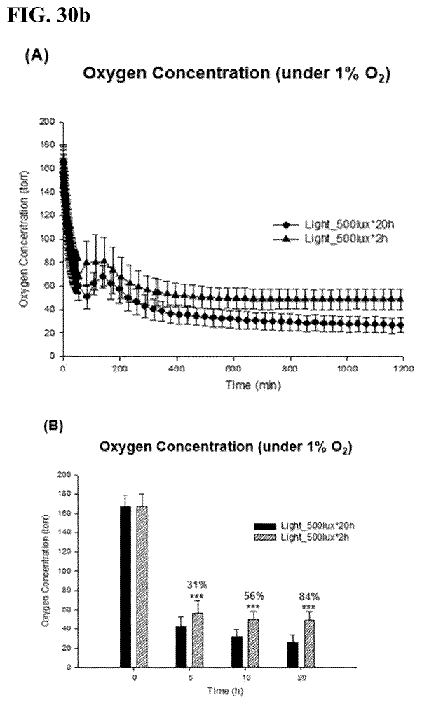

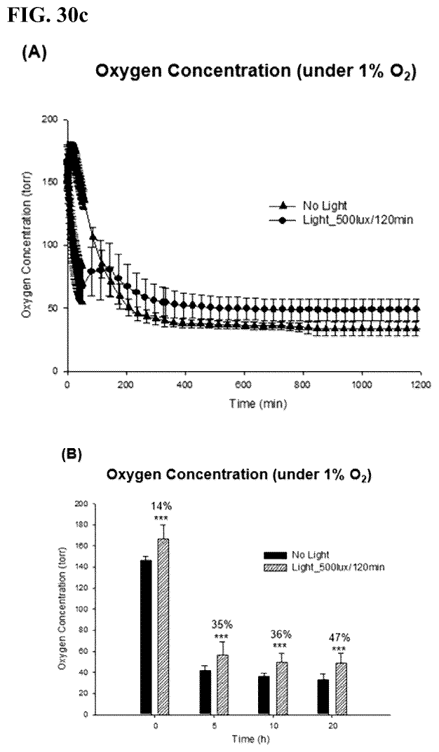

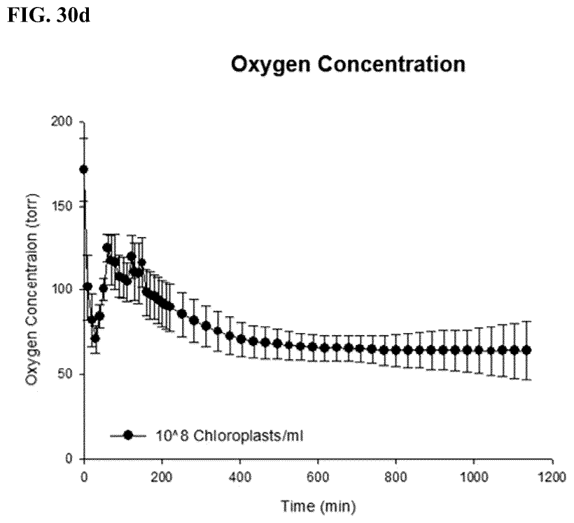

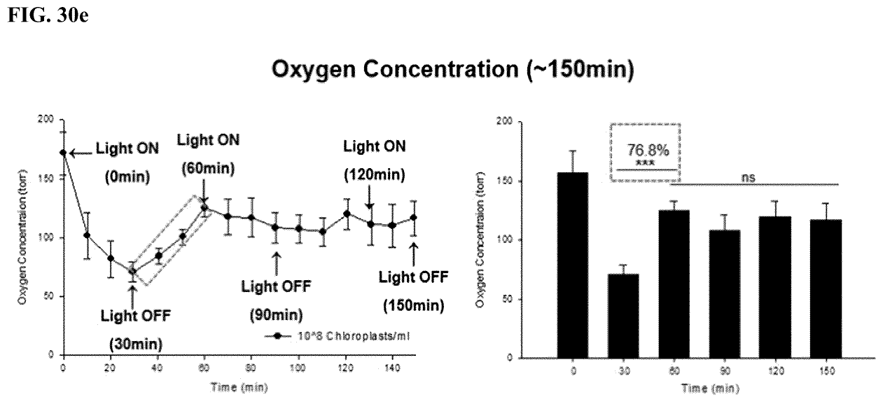

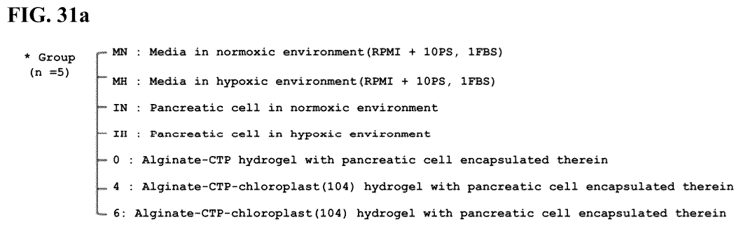

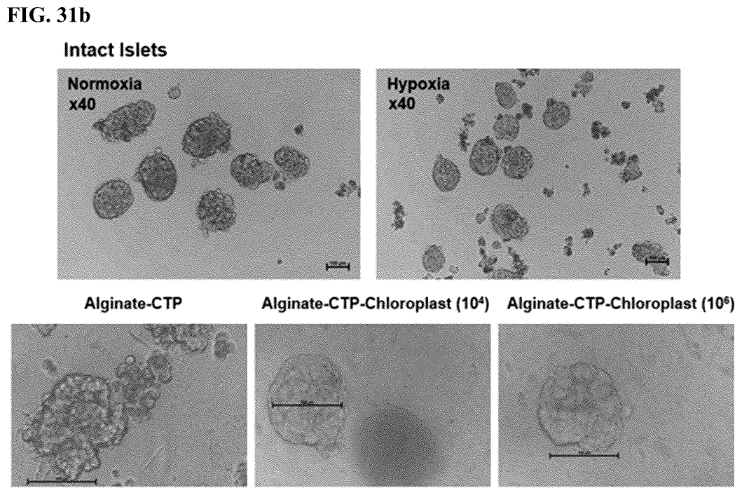

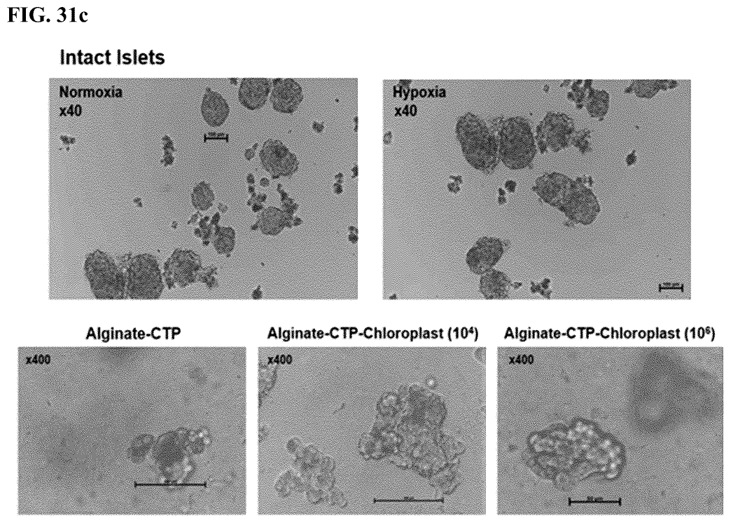

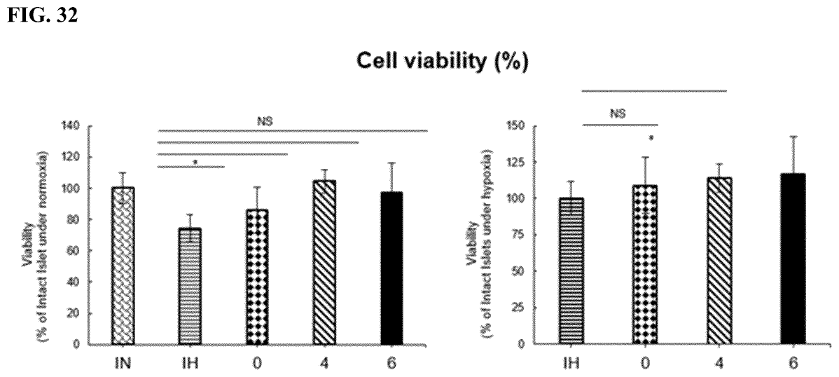

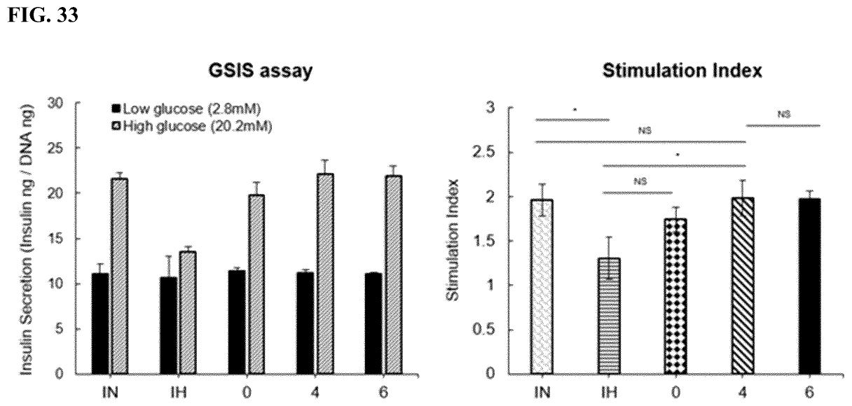

As a result, it can be confirmed that the initial oxygen concentration is increased as the intensity of light is decreased, and then tends to be almost constantly maintained for 20 h (see FIG. 30a). In addition, it is confirmed that an increase in the oxygen concentration by 20 mmHg for one hour, probably caused by a light effect, has a common pattern, representing that there is difference in the light effect according to the intensity of light, that is, an amount of oxygen generated by chloroplast. In other words, in a subsequent experiment, an exposure time of light was adjusted, not the intensity of light.

21.2.2. Measurement of Amount of Oxygen Generated According to Exposure Time of Light (660 nm)

An amount of oxygen generated from chloroplast in a hypoxic environment according to an exposure time of light (660 nm) was confirmed as follows.
Group (n=24)
Diluted with media (RPMI+10% FBS).
Concentration of chloroplast is fixed to 1*108/ml.
A temperature is fixed to 37° C.
An intensity of light (lux) is fixed at 500 lux.
(1) 20 hours
(2) 2 hours

Chloroplast at a concentration of 1*108/ml was dispensed by 1 ml each to an oxygen plate reader (24 wells). The plate was implemented in a hypoxic state as follows.
Hypoxic state: To implement a 1% oxygen, 37° C. environment by injecting a mixed gas (94% N2+5% CO2+1% O2) into a temperature-controlled desiccator.

A distance between material and light was set to 15 cm, and an intensity of light reaching the material was set to 500 lux. An amount of oxygen generated from chloroplast according to an exposure time of light was measured using an oxygen sensor. At this time, SDR SensorDish (PreSens, Germany) was used as the oxygen sensor.

As a result, it can be confirmed that the oxygen concentration decreases in the two groups as the exposure time of light increases (see FIG. 30b), and it can be confirmed that the time for maintaining the maximum oxygen concentration becomes longer in the group not exposed to the light after two hours compared to the group continuously exposed for 20 hours (see FIG. 30c). When the intensity of light is fixed at 500 lux, it can be confirmed that the maximum oxygen concentration is continued at 80 mmHg for about 100 minutes in the group exposed to light for two hours. In addition, when compared with the group not exposed to light under the same conditions (FIG. 30c), it was confirmed that when exposed to light for two hours, an amount of oxygen generated is increased by up to 47% compared with the group not exposed to light. It may be probably caused by photoinhibition, and it is estimated that when chloroplast is continuously exposed to strong light, active oxygen (ROS) is generated in photosystem II, in which photosynthesis occurs, and the oxygen generation ability of chloroplast is reduced. On the contrary, based on the result of a study that an amount of oxygen generated is increased due to the occurrence of reversible photoinhibition when chloroplast is exposed to light and then not exposed, a comparison was made on the amount of oxygen generated when chloroplast was exposed to light at time intervals in a subsequent experiment.

21.2.3. Measurement of Amount of Oxygen Generated According to an Exposure Time Interval of Light (660 nm)

An amount of oxygen generated from chloroplast in a hypoxic environment according to an exposure time interval of light (660 nm) was confirmed as follows.
Group (n=24)
Diluted with media (RPMI+10% FBS).
Concentration of chloroplast is fixed to 1*108/ml.
A temperature is fixed to 37° C.
An intensity of light (lux) is fixed at 500 lux.
(1) 30-minute exposure*three times

Chloroplast at a concentration of 1*108/ml was dispensed by 1 ml each to an oxygen plate reader (24 wells). The plate was implemented in a hypoxic state as follows.
Hypoxic state: To implement a 1% oxygen, 37° C. environment by injecting a mixed gas (94% N2+5% CO2+1% O2) into a temperature-controlled desiccator.

A distance between material and light was set to 15 cm, and an intensity of light reaching the material was set to 500 lux. After that, light was exposed three times for 30 minutes each and an amount of oxygen generated from chloroplast was measured using an oxygen sensor. At this time, SDR SensorDish (PreSens, Germany) was used as the oxygen sensor.

As a result of measuring an amount of oxygen generated for 20 hours after exposing chloroplast to light three times at a time interval of 30 minutes, it was found that the oxygen concentration is highly maintained at about 70-80 mmHg compared to the group not exposed to light (see FIGS. 30d and 30e). It could be confirmed that an oxygen increasing effect of about 76.8% occurs in the non-exposed state (after 30 minutes of exposure to light), compared to when being exposed to light. This is probably because the oxygen increasing effect does not occur due to the reversible photoinhibition of chloroplast in the state of exposure to light, but the oxygen generating effect starts to occur due to the stimulation of light while the damage of chloroplast is recovered when the exposure of light is removed (dark condition). In other words, it was confirmed that the oxygen generation of chloroplast is significantly increased to a maximum of 79.5% when being exposed to the light three times for 30 minutes each at a time interval (a total of 90 minutes) compared to when being continuously exposed to light for two hours. It was confirmed that there is an oxygen increasing effect of up to 47% compared to the group not exposed to light. Accordingly, a subsequent experiment was performed by exposing to light twice for 30 minutes each at a time interval for a total of 60 minutes with the light intensity of 1000 lux to maximize the stimulation of light.

### Example 22. Preparation of Alginate-CTP-Chloroplast Microcapsule

An alginate-CTP-chloroplast microcapsule was prepared as follows.
Group (n=5)
Diluted with CIB.
A synthesis ratio was fixed at a molar ratio of
alginate:peptide=1:1.
(1) Alginate microcapsule
(2) Alginate-chloroplast (2.5*109/ml) microcapsule
(3) Alginate-CTP-chloroplast (2.5*109/ml) microcapsule

Chloroplast diluted in CIB was prepared at a concentration (2.5*109/ml), and 1.5% alginate and alginate-CTP solution were prepared. 3% alginate solution and tertiary distilled water were mixed at a ratio of 1:1, 1.75 ml of chloroplast and 1.75 ml of 3% (w/v) alginate solution were added thereto and mixed, and then 1.75 ml of chloroplast and 1.75 ml of 3% (w/v) alginate-CTP solution were added thereto and mixed. After that, microcapsules for each group were prepared using an encapsulator (Nozzle size: 450 μm, Frequency: 800 Hz, Electrode: 500V, Flow rate: 15 ml/min), added to a gelation bath containing 30 ml of 100 mM CaCl2), and crosslinked by stirring for 15 minutes (stirring rate: 20 rpm). After that, the CaCl2) solution was removed, and the microcapsules were washed with 10 ml of media (RPMI+10% FBS+1% PS). 500 μl of microcapsules (about 500 units) for each group as well as 500 μl of media were placed in 1 well of 24-well plate.

As a result, it was confirmed that microcapsules are well formed in all groups.

### Example 23. Evaluation of Degradation and Mechanical Strength of Alginate-CTP-Chloroplast Microcapsule

The degradation and mechanical strength of alginate-CTP-chloroplast microcapsule were evaluated as follows.
Group (n=5)
Diluted with CIB.
A synthesis ratio was fixed at a molar ratio of alginate:peptide=1:1.
(1) Alginate microcapsule
(2) Alginate-chloroplast (2.5*109/ml) microcapsule
(3) Alginate-CTP-chloroplast (2.5*109/ml) microcapsule

Microcapsules for each group were prepared by being incubated in 24-well plate for 120 hours. A degree of decomposition was confirmed by examining an overall shape and size of microcapsules with the naked eye, and the strength of microcapsules for each group was confirmed by measuring rheology through a rheometer.

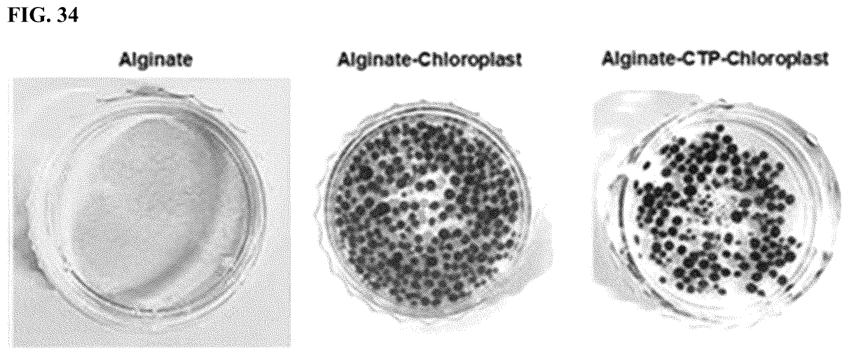

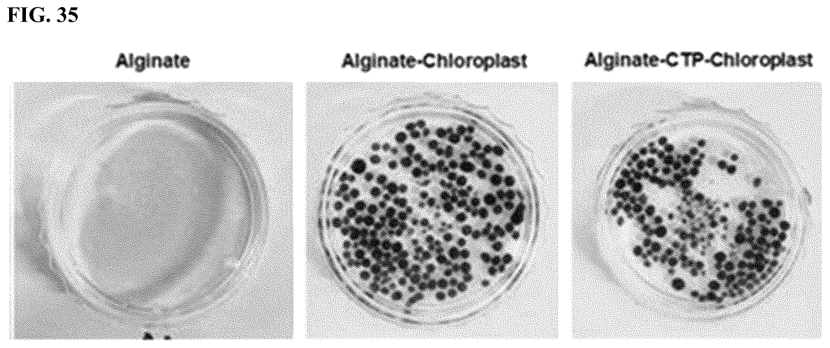

As a result of evaluating the degree of degradation for each group, it was confirmed that the microcapsules are well maintained even after 120 hours in all groups, representing that the microcapsules may be maintained for at least five days (see FIG. 35).

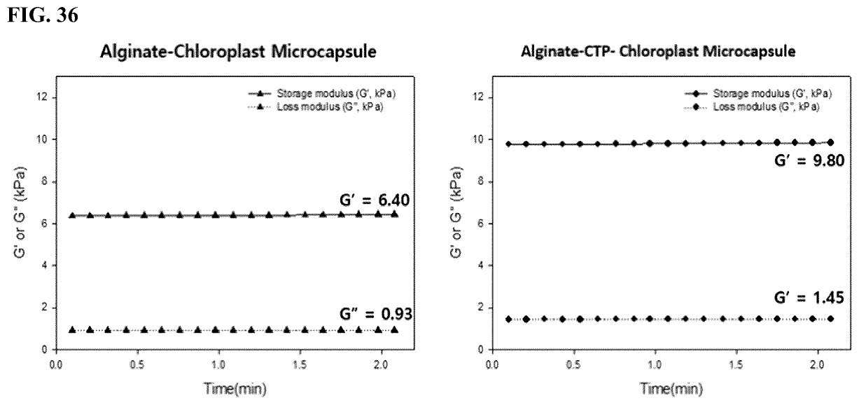

In addition, as a result of evaluating a mechanical strength for each group, it was confirmed that an elastic modulus (storage modulus, G) in the alginate-chloroplast group is 6.40 kPa, and an elastic modulus in the alginate-CTP-chloroplast group is 9.80 kPa (see FIG. 36). A value of the elastic modulus was higher in the group with the CTP synthesized, representing that less deformation occurs in the alginate-CTP-chloroplast group along with rigid physical properties.

### Example 24. Evaluation of Oxygen Control Ability of Alginate-CTP-Chloroplast Microcapsule

An amount of oxygen generated from alginate-CTP-chloroplast hydrogel microcapsule was confirmed as follows.
Group (n=6, diluted with CIB).
(1) Control (CIB)
(2) Chloroplast 1*106/ml
(3) Chloroplast 1*107/ml
(4) Chloroplast 1*108/ml
(5) Control (alginate microcapsule)
(6) Alginate-chloroplast 1*106/ml microcapsule:
(7) Alginate-chloroplast 1*107/ml microcapsule:
(8) Alginate-chloroplast 1*108/ml microcapsule:
A synthesis ratio of (9) to (11) was fixed at a molar ratio of alginate:peptide=1:1.
(9) Control (alginate-CTP microcapsule)
(10) Alginate-CTP-chloroplast 1*106/ml microcapsule
(11) Alginate-CTP-chloroplast 1*107/ml microcapsule
(12) Alginate-CTP-chloroplast 1*108/ml microcapsule

Chloroplast solution, alginate-chloroplast microcapsule, and alginate-CTP-chloroplast for each concentration corresponding to each group were prepared. Each group was put into an oxygen plate reader well plate (24 well) as follows, and an amount of oxygen generated was measured using an oxygen sensor while incubation was performed at 24-well plate for 48 hours. At this time, SDR SensorDish (PreSens, Germany) was used as the oxygen sensor.
Groups (1), (2), (3), (4): 500 μl of chloroplast solution for each concentration+500 μl of media (RPMI)
Groups (5), (6), (7), (8): 500 μl of alginate-chloroplast microcapsules for each concentration+500 μl of media (RPMI)
Groups (9), (10), (11), (12): 500 μl of alginate-CTP-chloroplast microcapsules for each concentration+500 μl of media (RPMI)

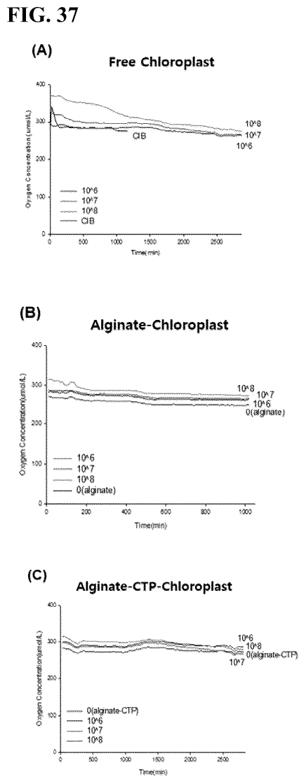

As a result, it was confirmed that an oxygen generation rate is the highest in the group of 108/ml having a high concentration of chloroplast in all groups, and it was confirmed that CTP has a relatively higher amount of oxygen generated in the group with the CTP synthesized, representing that the CTP has an effect of helping oxygen generation (see FIG. 37). In addition, an amount of oxygen generated was similar in chloroplast diluted in media (RPMI+10% FBS, 1% PS) rather than in chloroplast diluted in CIB, but due to the properties of the experiment using an encapsulator, when chloroplast was diluted using media (RPMI+10% FBS, 1% PS), the shape of microcapsule was not constant, and thus chloroplast was diluted using CIB to prepare a microcapsule.

### Example 25. Preparation of Alginate-CTP-Chloroplast Microcapsule with Pancreatic Cell Encapsulated Therein

The pancreatic cells were encapsulated in alginate-CTP-chloroplast microcapsule as follows.
Group (n=6, diluted with CIB, and a synthesis ratio was fixed at a molar ratio of alginate:peptide=1:1).
(1) Alginate microcapsule
(2) Alginate-chloroplast (2.5*109/ml) microcapsule
(3) Alginate-CTP-chloroplast (2.5*109/ml) microcapsule

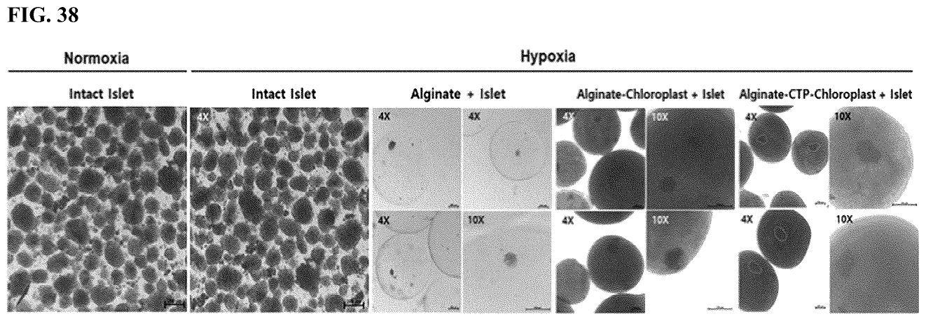

The pancreatic cells, which were isolated from an experiment animal (rat), were washed twice with media (RPMI). Chloroplast diluted in CIB was prepared at a concentration (2.5*109/ml), and 1.5% alginate and alginate-CTP solution were prepared. After that, 3% alginate solution and tertiary distilled water were mixed at a ratio of 1:1, 1.75 ml of chloroplast and 1.75 ml of 3% (w/v) alginate solution were added thereto and mixed, and then 1.75 ml of chloroplast and 1.75 ml of 3% (w/v) alginate-CTP solution were added thereto and mixed. Each 500 IEQ of extracted pancreatic cells was added to the mixed solution and mixed well. After that, microcapsules for each group were prepared using an encapsulator (* Nozzle size: 450 μm, * Frequency: 800 Hz, Electrode: 500V, Flow rate: 15 ml/min), added to a gelation bath containing 30 ml of 100 mM CaCl2), and crosslinked by stirring for 15 minutes (stirring rate: 20 rpm). After that, the CaCl2) solution was removed, and the microcapsules were washed with 10 ml of media (RPMI++10% FBS+1% PS). 500 μl of microcapsules (about 500 units) for each group as well as 500 μl of media were placed in 1 well of 24-well plate,
added to 300 μl of 100 mM CaCl₂) solution, and reacted, so as to confirm whether the pancreatic cells are well encapsulated in each group by using an optical microscope (see FIG. 38). As a result, it was confirmed that the pancreatic cells are well encapsulated in microcapsules in all groups except for the group not encapsulated in microcapsules.

### Example 26. Evaluation of Toxicity of Pancreatic Cell Encapsulated in Alginate-CTP-Chloroplast Microcapsule in Hypoxic Environment

26.1. Evaluation of Toxicity Through CCK-8 Assay

It was performed to confirm the viability of pancreatic cells encapsulated in alginate-CTP-chloroplast microcapsule in a hypoxic environment as follows.
Group (n=5, diluted with CIB, and a synthesis ratio was fixed at a molar ratio of alginate:peptide=1:1).
(1) Control 1 (RPMI media): Normoxic environment
(2) Control 2 (RPMI media): Hypoxic environment
(3) Pancreatic cell: Normoxic environment
(4) Pancreatic cell: Hypoxic environment
(5) Alginate microcapsule: Hypoxic environment
(6) Alginate-chloroplast (2.5*109/ml) microcapsule: Hypoxic environment
(7) Alginate-CTP-chloroplast (2.5*109/ml) microcapsule: Hypoxic environment

Pancreatic cells for each group, which were incubated in 24-well plate in normoxic and hypoxic states for 24 hours as well as microcapsules with the pancreatic cells encapsulated therein were prepared as follows.
Normoxic state: Typical 21% oxygen environment
Hypoxic state: 1% oxygen, 37° C. environment by injecting a mixed gas (94% N2+5% CO2+1% O2) into a temperature-controlled desiccator experiment instrument

All the pancreatic cells and microcapsules were washed twice with PBS, treated with 1 ml of media (RPMI)+100 μl of CCK solution, and incubated by wrapping the foil for two hours, after which absorbance was measured at 450 nm using a reader.

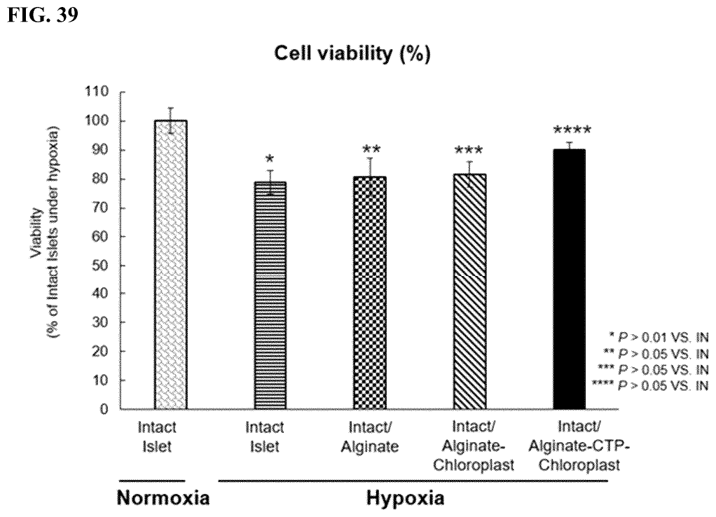

As a result, due to exposure of pancreatic cells to a hypoxic environment for a long time, viability of pancreatic cells was significantly reduced to about 22% compared to pancreatic cells in a normoxic environment (see FIG. 39), representing that a hypoxic environment is not an environment appropriate for pancreatic cells to survive. As a result of confirming viability of pancreatic cells encapsulated in alginate-CTP-chloroplast microcapsules, there was a reduction by about 10% compared to the pancreatic cells in a normoxic environment, representing that the alginate-CTP-chloroplast material may help the survival of pancreatic cells. When comparing an alginate-chloroplast microcapsule with an alginate microcapsule, it was confirmed that the chloroplast of the alginate-chloroplast microcapsule may produce oxygen which may help the survival of the cells. When comparing an alginate-CTP-chloroplast microcapsule with an alginate-chloroplast microcapsule, the reduction of cell viability was lowered in the alginate-CTP-chloroplast microcapsule, representing that the efficacy of CTP plays an important role in the production of oxygen by chloroplast, and thus that CTP-stabilized chloroplast has a more advantage in terms of functions. When comparing the cell viability for each group, the cell viability of the pancreatic cells encapsulated in the alginate-CTP-chloroplast microcapsule was lower than that of the pancreatic cells in the normoxic environment, but this is probably affected by metabolites (metabolome) or reactive oxygen (ROS) of the pancreatic cells present in the alginate-CTP-chloroplast microcapsule. Accordingly, in order to compensate for this problem in subsequent experiments, an experiment was performed by synthesizing catalase (CAT) or superoxide dismutase (SOD) enzyme.

26.2. Evaluation of Toxicity Through Live & Dead Assay

The viability of pancreatic cells encapsulated in alginate-CTP-chloroplast microcapsule in a hypoxic environment was confirmed as follows.
Group (n=5, diluted with CIB, and a synthesis ratio was fixed at a molar ratio of alginate:peptide=1:1).
(1) Control 1 (RPMI media): Normoxic environment
(2) Control 2 (RPMI media): Hypoxic environment
(3) Pancreatic cell: Normoxic environment
(4) Pancreatic cell: Hypoxic environment
(5) Alginate microcapsule: Hypoxic environment
(6) Alginate-chloroplast (2.5*109/ml) microcapsule: Hypoxic environment
(7) Alginate-CTP-chloroplast (2.5*109/ml) microcapsule: Hypoxic environment

Pancreatic cells for each group, which were incubated in 24-well plate in the following normoxic and hypoxic states for 24 hours as well as microcapsules with the pancreatic cells encapsulated therein were prepared.
Normoxic state: Typical 21% oxygen environment
Hypoxic state: 1% oxygen, 37° C. environment by injecting a mixed gas (94% N2+5% CO2+1% O2) into a temperature-controlled desiccator experiment instrument

All the pancreatic cells and microcapsules were washed twice with PBS, after which each well was treated with 1 ml of a reagent prepared with 5 μM calcein-AM and 20 μM ethidium homodimer-1 (EthD-1), and incubated for 40 minutes. At this time, groups (3) and (4) were performed for 15 minutes. After that, live cells (green fluorescence) and dead cells (red fluorescence) were confirmed using a fluorescence microscope, and then a photograph was taken.

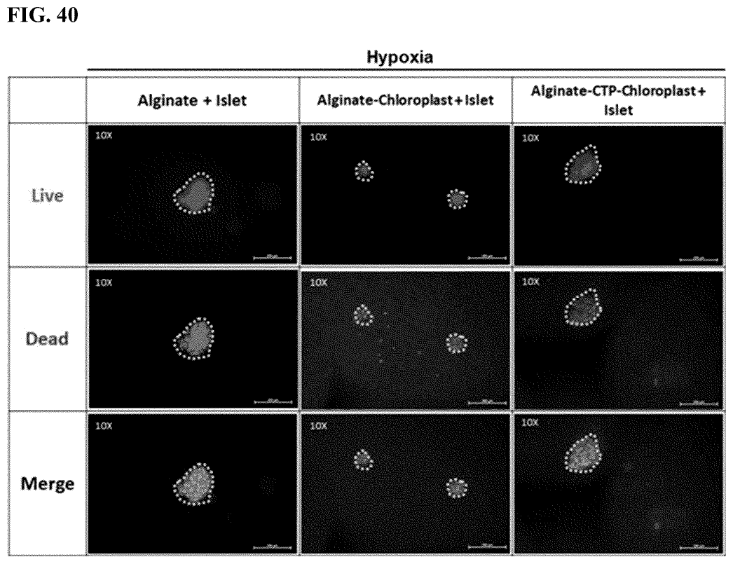

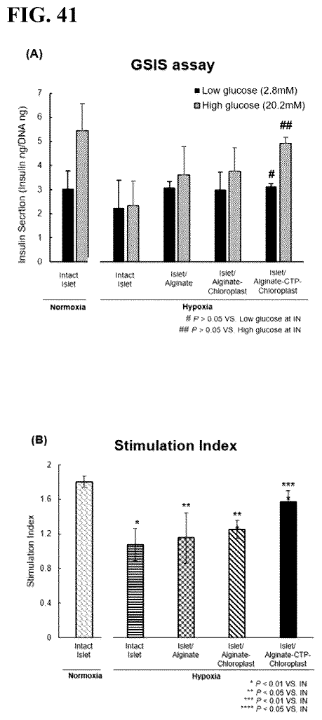

As a result, it was confirmed that a green signal appears to be relatively strong in the alginate-CTP-chloroplast microcapsule group compared to other groups in a hypoxic environment, and a yellow signal appears to be the weakest as a result of the combination of green and red (see FIG. 40). It means that the alginate-CTP-chloroplast microcapsule is an environment appropriate for pancreatic cells to survive, as in the above CCK-8 assay result.

### Example 27. Confirmation of Functionality of Pancreatic Cell Encapsulated in Alginate-CTP-Chloroplast Microcapsule in Hypoxic Environment

27.1. Confirmation of Functionality Through Glucose-Stimulated Insulin Secretion (GSIS) Assay

An insulin secretion function of pancreatic cells encapsulated in alginate-CTP-chloroplast microcapsule in a hypoxic environment was confirmed as follows.

The materials included (1) low glucose (2.8 mM, diluted with Krebs Ringer buffered HEPES (pH 7.4)) and (2) high glucose (20.2 mM, diluted with Krebs Ringer buffered HEPES (pH 7.4)).
Group (n=5, diluted with CIB, and a synthesis ratio was fixed at a molar ratio of alginate:peptide=1:1).
(1) Control 1 (RPMI media): Normoxic environment
(2) Control 2 (RPMI media): Hypoxic environment
(3) Pancreatic cell: Normoxic environment
(4) Pancreatic cell: Hypoxic environment
(5) Alginate microcapsule: Hypoxic environment
(6) Alginate-chloroplast (2.5*109/ml) microcapsule: Hypoxic environment
(7) Alginate-CTP-chloroplast (2.5*109/ml) microcapsule: Hypoxic environment

Pancreatic cells for each group, which were incubated in 24-well plate in normoxic and hypoxic states for 24 hours as well as microcapsules with the pancreatic cells encapsulated therein were prepared as follows.
Normoxic state: Typical 21% oxygen environment
Hypoxic state: 1% oxygen, 37° C. environment by injecting a mixed gas (94% N2+5% CO2+1% O2) into a temperature-controlled desiccator experiment instrument

All the pancreatic cells and microcapsules were washed twice with PBS, and then low glucose (2.8 mM) was dispensed by 1 ml in each well and incubated for 30 minutes. Low glucose (2.8 mM) was again dispensed by 1 ml to each well and incubated for two hours, and then the present low glucose solution was recovered. After that, high glucose (20.2 mM) was dispensed by 1 ml to each well and incubated for two hours, and then the high glucose solution present was recovered. An amount of insulin was measured and calculated based on low glucose and high glucose solutions recovered using insulin enzyme-linked immunosorbent (ELISA) assay.

Then, as a result of comparing the pancreatic cells in a normoxic environment with the pancreatic cells in a normoxic environment, it was confirmed that an insulin secretion ability of the pancreatic cells is affected by the surrounding environment. It means that a hypoxic environment is not an environment appropriate for pancreatic cells to survive. It was confirmed that the pancreatic cells encapsulated in alginate-CTP-chloroplast microcapsules secrete insulin at a level similar to that of pancreatic cells of normoxia at both low and high glucose concentrations, representing that the alginate-CTP-chloroplast microcapsule material does not affect the insulin secretion ability of encapsulated pancreatic cells. It was confirmed that the pancreatic cells encapsulated in alginate-CTP-chloroplast microcapsules have an insulin secretion ability at the most similar level to that of pancreatic cells of normoxia among hypoxic environment experimental groups, representing that the alginate-CTP-chloroplast microcapsule material may best maintain an insulin secretion ability of encapsulated pancreatic cells. It was confirmed that the pancreatic cells encapsulated in the alginate-CTP-chloroplast microcapsule have a significantly higher glycemic control function than that of the pancreatic cells encapsulated in the alginate-chloroplast microcapsule, and the pancreatic cells encapsulated in the alginate-CTP-chloroplast microcapsule maintain a glycemic control function compared to the pancreatic cells encapsulated in the alginate-chloroplast microcapsule. The pancreatic cells encapsulated in the alginate-CTP-chloroplast microcapsules have the highest ability to secrete insulin among respective groups in a hypoxic environment, representing that the alginate-CTP-chloroplast microcapsule material has the potential to increase the viability of pancreatic cells and the potential to maintain the ability thereof from a hypoxic environment. An amount of insulin secreted from the pancreatic cells encapsulated in the alginate-CTP-chloroplast microcapsule was lower than that of the pancreatic cells in the normoxic environment, but this is probably affected by metabolites (metabolome) or reactive oxygen (ROS) of the pancreatic cells present in the alginate-CTP-chloroplast microcapsule. Accordingly, in order to compensate for this problem in subsequent experiments, an experiment was performed by synthesizing catalase (CAT) or superoxide dismutase (SOD) enzyme.

27.2. Confirmation of Functionality Through dsDNA Assay

An amount of insulin secreted from the pancreatic cells encapsulated in alginate-CTP-chloroplast microcapsule in a hypoxic environment was corrected with a size of pancreatic cells as follows.
Group (n=5, diluted with CIB, and a synthesis ratio was fixed at a molar ratio of alginate:peptide=1:1).
(1) Control 1 (RPMI media): Normoxic environment
(2) Control 2 (RPMI media): Hypoxic environment
(3) Pancreatic cell: Normoxic environment
(4) Pancreatic cell: Hypoxic environment
(5) Alginate microcapsule: Hypoxic environment
(6) Alginate-chloroplast (2.5*109/ml) microcapsule: Hypoxic environment
(7) Alginate-CTP-chloroplast (2.5*109/ml) microcapsule: Hypoxic environment

Pancreatic cells for each group, which were incubated in 24-well plate in normoxic and hypoxic states for 24 hours as well as microcapsules with the pancreatic cells encapsulated therein were prepared as follows.
Normoxic state: Typical 21% oxygen environment
Hypoxic state: 1% oxygen, 37° C. environment by injecting a mixed gas (94% N2+5% CO2+1% O2) into a temperature-controlled desiccator experiment instrument

All the pancreatic cells and microcapsules were washed twice with PBS, and 0.1 M EDTA (pH 7.8) solution was dispensed by 1 ml into each well and the pancreatic cells and microcapsules were crushed through sufficient pipetting (in order to take out the pancreatic cells present in microcapsules). Each of the solution containing the crushed pancreatic cells and microcapsules was recovered into 1.5 ml tube and subjected to centrifugation at 1800 RPM for two minutes. The supernatant was removed and then pellets (pancreatic cells) were treated with 1 ml of RIPA buffer each. An amount of insulin secreted by pancreatic cells present in each well (alginate-CTP-chloroplast) was corrected through dsDNA assay.

### Example 28. Confirmation of Binding of Chloroplast Outer Membrane to CTP(VILGLGLAGI)-FITC Using Fluorescent Image

It was confirmed by fluorescence whether CTP(VILGLGLAGI)-FITC may be also anchored on an outer membrane of chloroplast in addition to CTP(MFAFQYLLVM) by the following method. Care was taken not to be exposed to light during the experiment.

500 μl of 1*108/ml chloroplast (control) and 500 μl of 1*108/ml chloroplast+CTP-FITC were prepared (CTP-FITC was obtained upon request from Peptron, a synthesis company. In 1*108/ml chloroplast+CTP-FITC, CTP-FITC was treated at a concentration of 0.6 mg/ml. If 500 μl of 1*108/ml chloroplast (in CIB) is taken and 30 μl of 10 mg/ml CTP (in DMSO) is taken and added, the final concentration may become 0.6 mg/ml. After that, 100 μl was taken and immobilized every 1, 5, 10, 30 and 60 minutes, and the solution reacted for each time was subjected to centrifugation at 13500 RPM for one minute. A pellet was obtained, immobilized in a 2.5% glutaraldehyde (0.05 M SCB) fixative (500 μl, 15 min), and washed twice with PBS to remove unreacted CTP-FITC. After that, 500 μl was filled with CIB buffer, and then mounted using slide glass/cover glass. A fluorescent photograph was taken using a confocal microscope, and sectioning was performed with the thickness of about 1 μm (green fluorescence (FITC)=488 nm, red fluorescence (chloroplast auto fluorescence)=580 nm).

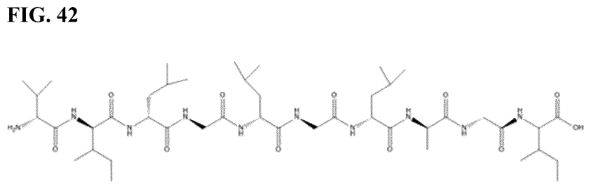

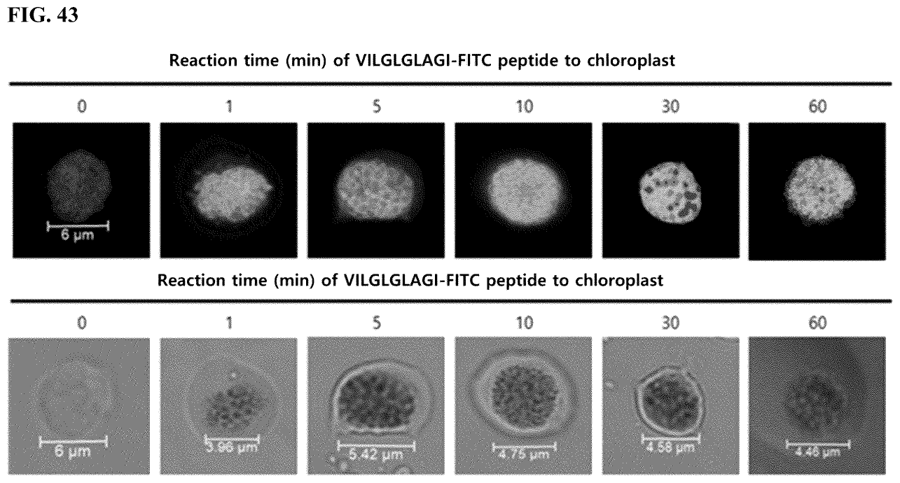

As a result, it could be confirmed that CTP(V-) is anchored onto an outer membrane of chloroplast from a reaction time of five minutes or more to be sufficiently coated. In addition, it was confirmed that anchoring is possible up to an inner membrane, and it was confirmed that CTP(V-) stably surrounds chloroplast (see FIG. 43).

### Example 29. Alginate-CTP (VILGLGLAGI) Conjugation

### 29.1. Alginate-CTP (VILGLGLAGI) Synthesis

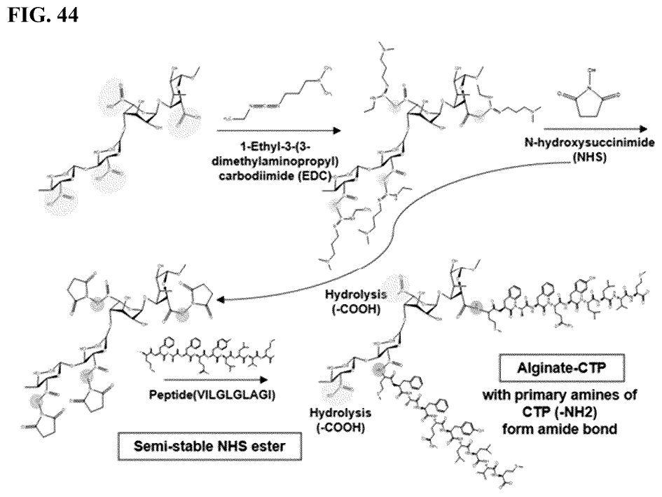

Carboxylate (—COOH) group of alginate and primary amine (—NH2) of CTP(V-) were synthesized by the following method. At this time, CTP(VILGLGLAGI) was used instead of CTP(MFAFQYLLVM). A carbodiimide coupling reaction was used to form an amide bond with carboxylate (—COOH) group of alginate and primary amine of CTP(V-) to prepare a conjugate. EDC/NHS was used to increase the efficiency of the carbodiimide coupling reaction (see FIG. 44).

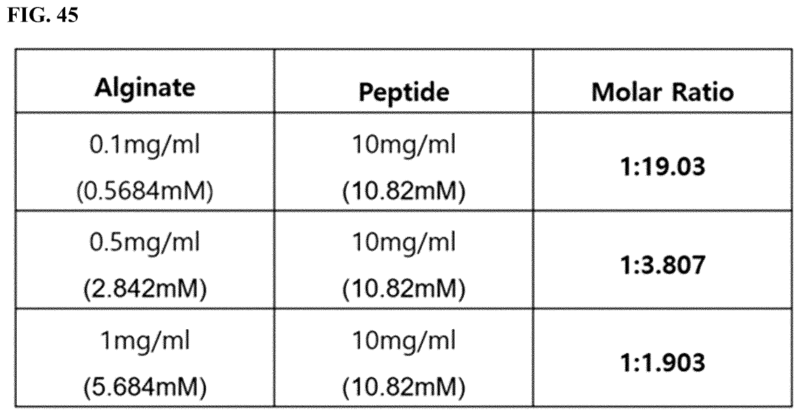

After preparing 10 ml of 0.1 M MES buffer (+0.5 M NaCl), pH was adjusted to 6. 0.088 mM sodium alginate (10 mg/ml) was dissolved in 10 ml of the prepared MES buffer, so that sodium alginate might be well loosen and might not be agglomerated. When the solution was sufficiently dissolved, 4 mg/10 ml EDC (191.7 M.W) corresponding to 2 mM was dissolved. After that, 6 mg/10 ml NHS (5 mM) was added thereto, reacted at RT for 15 minutes, and titrated to pH 7 with PBS or sodium bicarbonate (NaHCO₃, sodium hydrogen carbonate). Peptide (10 mg/ml, peptide was synthesized upon request by Peptron, a synthesis company) dissolved in 1% DMSO was added to the solution in an amount corresponding to a molar ratio (1:1.9, 1:3.8, 1:19) and reacted at RT for two hours. The resulting mixture was subjected to dialysis for three days using a dialysis membrane with a molecular weight cut-off (MWCO) of 6,000 to 8,000. After that, the resulting product was frozen in a deep freezer, and then freeze-dried to obtain an alginate-CTP(VILGLGLAGI) composite in the form of powder (see FIG. 45). Sodium alginate is a polymer in which 648 M carboxylate group (—COOH) is present in 1 M. Since there are many carboxylate groups (—COOH) present in 1 molecule of alginate, an experiment was performed at a reduced concentration of sodium alginate compared to a ratio which was applied to synthesize the existing chloroplast-transit-peptide (CTP(V-)) and at the same concentration of peptide as 10 mg/ml.

29.2. Confirmation of Alginate-CTP (VILGLGLAGI) Conjugation

29.2.1. CONFIRMATION THROUGH FT-IR

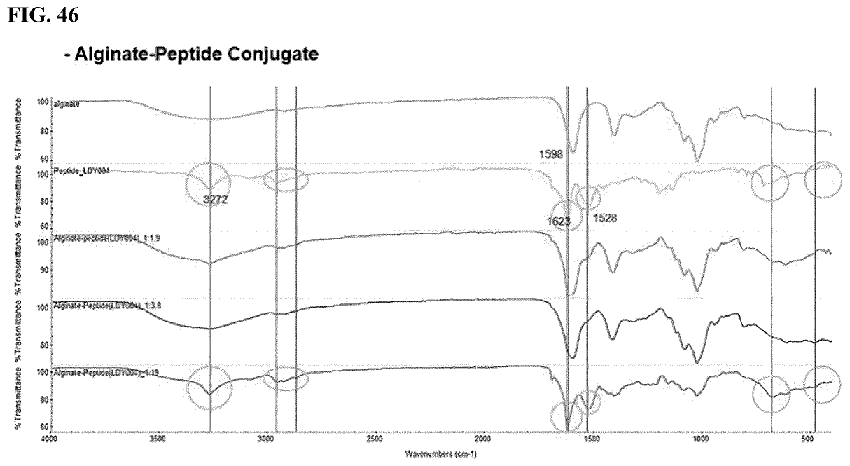

A conjugate having a ratio of alginate and peptide at 1:19 well showed the properties of peptide because alginate (0.1 mg/ml) was the least contained (see FIG. 46). When alginate and peptide were synthesized, a new amide bond was generated by a new EDC/NHS reaction. An amine group has several peaks, among which even in the peptide, there is a primary amine peak having amide A and B bonds (NH stretch). Thus, in order to confirm a new amide bond of a composite, it was confirmed whether a peak (1480-1575 cm-1) of amide II (C—N) bond is present or not. As a result, the amide II (C—N) bond appeared at 1:19 and peptide, and may be well seen due to a relatively large number of peptides. However, when comparing the intensity of peak in a corresponding region, it could be confirmed that it was stronger in the conjugate of 1:19.

29.2.2. Confirmation Through 1H-NMR

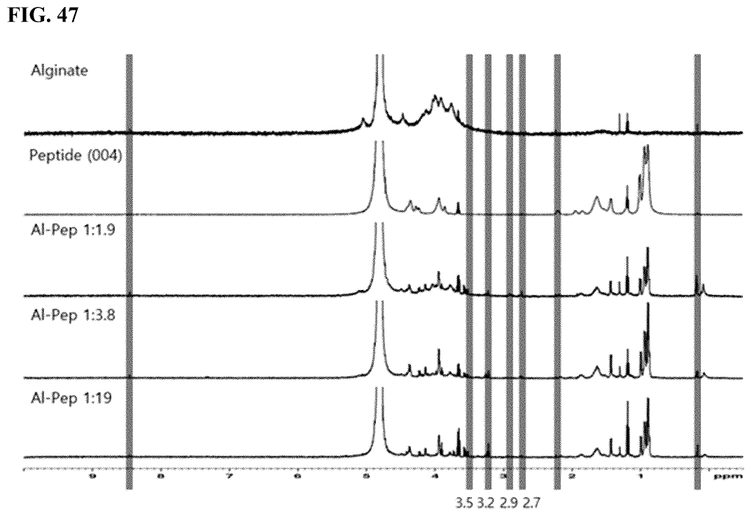

A conjugate having a ratio of alginate and peptide at 1:19 well show the properties of peptide because alginate (0.1 mg/ml) is the least contained (see FIG. 46). When alginate and peptide are synthesized, a new amide bond needs to be generated by a new EDC/NHS reaction. 2.2 ppm is a peak which mainly appears in the peptide, and is a peak which is expected to be formed by a primary amine (N-terminal). It was confirmed that the peak appears stronger as an amount of peptide increases according to a relative binding ratio (see FIG. 47). In other words, it can be seen that the properties of peptide are relatively well represented as the peak in 2.2 ppm increases, and it can be determined that the peak becomes stronger due to the primary amine originally present in the peptide due to a new amide bond. 3.2 ppm is a peak in which both alginate and peptide do not appear, and it can be confirmed that peak is strongly formed as the peptide concentration increases. When compared with an FTIR result, it can be expected that amide bond II (C—N) is formed in 3.2 ppm, and it can be estimated that alginate carboxylate group (—COOH) is changed into secondary amine by binding to primary amine of peptide.

The above description of the present invention is for illustration, and those skilled in the art to which the present invention pertains will understand that the present invention can be easily modified into other specific forms without departing from the technical idea or essential features of the present invention. Thus, it should be understood that the exemplary embodiments described above are illustrative in all aspects and are not contrived to limit the scope of the present invention.

1. A hydrogel composition comprising a chloroplast and a chloroplast transit peptide (CTP).

2. The hydrogel composition of claim 1, wherein the CTP binds to a chloroplast outer membrane.

3. The hydrogel composition of claim 1, wherein the CTP is derived from a chloroplast outer envelope protein.

4. The hydrogel composition of claim 3, wherein the chloroplast outer envelope protein is outer envelope protein 34 (OEP34) or outer envelope protein 64 (OEP64).

5. The hydrogel composition of claim 1, wherein the CTP comprises one or more selected from amino acid sequences represented by SEQ ID NO: 1 and SEQ ID NO: 2.

6. The hydrogel composition of claim 1, wherein the CTP increases oxygen generation of the chloroplast.

7. The hydrogel composition of claim 1, wherein the hydrogel composition further comprises alginate.

8. The hydrogel composition of claim 7, wherein the hydrogen is gelated by binding a CTP terminus and an alginate terminus.

9. The hydrogel composition of claim 1, wherein the hydrogel further comprises a pancreatic cell.

10. The hydrogel composition of claim 1, wherein the hydrogel composition generates oxygen upon light irradiation.

11. The hydrogel composition of claim 10, wherein the light irradiation is performed at an oxygen concentration of 10¹/ml to 10¹⁰/ml.

12. The hydrogel composition of claim 10, wherein the light irradiation is performed at a temperature of 20° C. to 40° C.

13. The hydrogel composition of claim 10, wherein the light irradiation is performed one to five times for 10 to 50 minutes at intervals of 10 to 50 minutes.

14. The hydrogel composition of claim 1, wherein the hydrogel comprises chloroplasts at a concentration of 10¹/ml to 10¹⁰/ml.

15. The hydrogel composition of claim 9, wherein the hydrogel comprises insulin secreted by the pancreatic cell.

16. A hydrogel composition, wherein the hydrogel composition of claim 1 is for delivering oxygen to a cell or a tissue.

17. A microcapsule comprising the hydrogel composition of claim 1.

18. A method of preventing or treating a metabolic disease, comprising administering a hydrogel composition of claim 1 as an active ingredient to an individual.

19. A health functional food for preventing or ameliorating a metabolic disease, comprising the hydrogel composition of claim 1 as an active ingredient.

20. A method for preparing a hydrogel composition, the method comprising:
binding alginate and a chloroplast transit peptide (CTP); and
mixing the bound alginate-CTP composite with a chloroplast isolated from an individual to gelate the bound alginate-CTP composite.

21. The method of claim 20, further comprising:
encapsulating a pancreatic cell in the gelated hydrogel.

22. A method for delivering oxygen to a cell or a tissue, the method comprising:
treating the hydrogel composition of claim 1; and
irradiating the treated hydrogel composition with light.

23. The method of claim 22, wherein the irradiating with light is performed at an oxygen concentration of 10¹/ml to 10¹⁰/ml.

24. The method of claim 22, wherein the irradiating with light is performed at a temperature of 20° C. to 40° C.

25. The method of claim 22, wherein the irradiating with light is performed one to five times for 10 to 50 minutes at intervals of 10 to 50 minutes.
